(19) United States
(12) Patent Application Publication
(10) Pub. No.: US 2018/0070928 A1
(43) Pub. Date: Mar. 15, 2018

(54) Systems and Methods for Extracting a Sample from an Ingestible Device

(71) Applicant: Progenity Inc., San Diego, CA (US)

(72) Inventors: Mitchell Lawrence Jones, La Jolla, CA (US); Christopher Loren Wahl, La Jolla, CA (US); Aaron Olafur Laurence Philippsen, Victoria (CA); Matthieu Olivier Lemay, Victoria (CA); Nicholas David Allan, Victoria (CA)

(21) Appl. No.: 15/694,458

(22) Filed: Sep. 1, 2017

Related U.S. Application Data
(60) Provisional application No. 62/385,344, filed on Sep. 9, 2016.
(60) Provisional application No. 62/480,187, filed on Mar. 31, 2017.
(60) Provisional application No. 62/540,873, filed on Aug. 3, 2017.

(51) Int. Cl. A61B 10/00; A61B 10/02; A61B 5/07

(52) U.S. Cl. CPC A61B 10/0096; A61B 10/0045; A61B 2562/162; A61B 5/073; A61B 2010/0061; A61B 10/02

(57) ABSTRACT

Systems and methods for extracting and/or analyzing a sample, such as a sample present in an ingestible device, are disclosed.

### CROSS-REFERENCE TO RELATED APPLICATIONS

This application claims priority under 35 U.S.C. § 119 to: U.S. Provisional Patent Application 62/385,344, filed on Sep. 9, 2016, entitled “Systems and Methods for Extracting a Sample from an Ingestible Device;” U.S. Provisional Patent Application No. 62/480,187 filed on Mar. 31, 2017, entitled “Localization Systems and Method for an Ingestible Device;” U.S. Provisional Patent Application No. 62/540,873 filed on Aug. 3, 2017, entitled “Localization Systems and Method for an Ingestible Device”.

### INCORPORATION BY REFERENCE

This application incorporates by reference the following patent applications: U.S. Ser. No. 14/460,893; 15/514,413; 15/680,400; 15/680,430; 62/385,344; 62/385,553; 62/478,955; 62/434,188; 62/434,320; 62/431,297; 62/434,797; 62/480,187; 62/502,383; and 62/540,873.

### FIELD

The disclosure relates to systems and methods for extracting and/or analyzing a sample, such as a sample present in an ingestible device.

### BACKGROUND

The gastrointestinal (GI) tract generally contains a wealth of information regarding an individual's body. For example, contents in the GI tract may provide information regarding the individual's metabolism. An analysis of the contents of the GI tract may also provide information for identifying relationships between the GI content composition (e.g., relationship between bacterial and biochemical contents) and certain diseases and disorders.

### SUMMARY

The disclosure provides systems and methods for extracting samples from a device, such as an ingestible device. Alternatively, and/or additionally, the disclosure provides systems and methods for analyzing such samples. The samples can be taken while the device is in the GI tract of a subject.

The systems and methods can allow for relatively little contamination during sample collection, containment and/or extraction. For example, for samples contained in different sampling chambers within an ingestible device, there can be less than 5% contamination of either sample during the sample collection, sample containment and sample extraction.

In one aspect, the disclosure provides a method for extracting a sample from an ingestible device, the ingestible device having a housing with an opening connected to a sampling chamber of the ingestible device. The includes the ingestible device to transfer at least a portion of a sample contained in the sampling chamber out of the ingestible device and into a tube via the opening.

The method can further include connecting the opening to the tube via a sleeve.

The method can further include inserting at least a portion of the ingestible device into the sleeve.

The sleeve can be a fitted sleeve.

The ingestible device can be centrifuged while the ingestible device is at least partially inserted into the sleeve.

The method can further include generating the opening.

The method can further include exposing the ingestible device to a heated lancet to generate the opening.

The method can further include sliding a grating surface against the ingestible device to remove a portion of the housing of the ingestible device to generate the opening.

The ingestible device can have a second opening connected to a second sampling chamber of the ingestible device, and the method can further include centrifuging the ingestible device to transfer at least a portion of a second sample contained in the second sampling chamber into a second tube via the second opening.

The sampling chamber further can include an absorbent material, and the sample can be at least partially absorbed by the absorbent material prior to centrifuging.

Centrifuging can include: inserting at least a portion of the ingestible device into a sleeve; while at least the portion of the ingestible device is inserted into the sleeve, inserting the ingestible device and the sleeve into a centrifuge tube; attaching the centrifuge tube to a cap having a spring on a bottom surface of the cap; causing the spring to maintain a position of the ingestible device relative to the fitted sleeve as the ingestible device is at least partially inserted into the fitted sleeve; and centrifuging the centrifuge tube while the ingestible device and the fitted sleeve are inserted into the centrifuge tube.

The method can further include using a moveable screw positioned on a side of the centrifuge tube to restrict movement of the ingestible device relative to the centrifuge tube.

The housing of the ingestible device can include an indentation, and the opening can be present at the indentation.

The sample can be obtained by the ingestible device from a predetermined region of the gastrointestinal tract.

The method can result in less than 5% sample contamination.

In one aspect, the disclosure provides a sleeve device configured to extract a sample from an ingestible device comprising a housing with a first end having a device opening that leads to a sampling chamber containing the sample. The sleeve device includes: a sleeve portion configured to fit snugly around a circumference of the ingestible device; and a base portion. The base portion includes a first surface with a first opening; a second surface with a second opening; and a tunnel connecting the top and second openings. The first surface is connectable to the sleeve portion. The first surface comprises a seal portion configured to fit the first end of the ingestible device and to provide a liquidtight seal between the first opening and the device opening.

The first surface can be a side surface of the base portion.

The first surface can be a top surface of the base portion.

The sleeve device can further include a removable tube having a tube opening that faces the second opening when the removable tube is connected to the second surface of the base portion.

The removable tube can be connected to the second surface of the base portion by one or more magnets.

The sleeve portion and the base portion can be connected by a dowel joint.

The sleeve portion and the base portion can be connected by a mortise and a tenon joint.

The sleeve device can further include: a first dowel-shaped opening on the second surface; a second dowel-shaped opening on the first surface; and a first dowel-shaped tunnel connecting the first and second dowel-shaped openings; a third dowel-shaped opening on the end of the sleeve portion, the third dowel-shaped opening connecting to a second dowel-shaped tunnel extending into the sleeve portion; and a dowel within the first dowel shaped opening, the first dowel shaped tunnel, the second dowel shaped opening, the third dowel shaped opening, and the second dowel shaped tunnel.

The sleeve can further include a window located on a side of the sleeve portion. The window can be usable to align the ingestible device to the sleeve portion so that the first opening faces the device opening.

The sleeve device can further include: an aperture located on a side of the sleeve portion; and a positioning pin insertable into the aperture to fix an orientation of the ingestible device relative to the sleeve portion.

The base portion can further include: a second tunnel connecting a third opening on the first surface of the base portion to a fourth bottom opening on the second surface of the base portion; and a second seal portion that is sized and shaped to fit on the first end of the ingestible device and form a second watertight seal between the third and fourth openings.

The sleeve device can further include a second removable tube having a second tube opening that faces the fourth opening when the second removable tube is connected to the second surface.

The sleeve device can be configured to result in less than 5% sample contamination.

In one aspect, the disclosure provides a method that includes separating an ingestible device into a first portion and a second portion, the first portion comprising a sampling chamber that contains a sample. The method also includes centrifuging the first portion of the ingestible device to transfer at least a portion of the sample from the sampling chamber into the tube.

The method can further include, prior to centrifuging, connecting the sampling chamber to the tube via an adapter.

Separating the ingestible device into the first and second portions can include: inserting the ingestible device into a cutting device having a recessed area and a moveable blade such that the ingestible device is positioned within the recessed area; and causing the moveable blade to cut the ingestible device to form the first portion and the second portion while the recessed area holds the ingestible device.

Separating the ingestible device into the first and second portions can expose a barrier within the first portion of the ingestible device which defines a portion of an outer surface of the sampling chamber.

The method can further include inserting an adapter into the first portion of the ingestible device so that a hollow needle on the adapter penetrates the barrier and enters the sampling chamber.

The hollow needle can penetrate an indentation on the barrier.

centrifuging can transfer at least a second portion of the sample from the ingestible device into a second tube, the second portion of the sample contained in a second sampling chamber prior to being transferred into the second tube.

The portion of the sample can be absorbed by a sponge within the sampling chamber prior to being transferred into the tube.

The method can further include disconnecting the tube from the adapter after centrifuging the first portion of the ingestible device.

The method can further include: inserting the first portion of the ingestible device and the adapter into a centrifuge tube; attaching the centrifuge tube to a cap having a spring on a bottom surface of the cap; causing the spring to maintain a position of the first portion of the ingestible device relative to the adapter; and centrifuging the centrifuge tube while the first portion of the ingestible device and the adapter are inserted into the centrifuge tube.

The method can further include using a moveable screw positioned on a side of the centrifuge tube to restrict movement of the first portion of the ingestible device relative to the centrifuge tube.

The sample can be obtained by the ingestible device from a predetermined region of the gastrointestinal tract.

The method can result in less than 5% sample contamination.

In one aspect, the disclosure provides an adapter device having a first surface with a first opening, a second surface with a second opening, and a body between the first and second surfaces, the body having a tunnel that connects the first and second openings. The adapter device includes a hollow needle protruding from the second surface and having a lumen connected to the second opening to define a fluid channel from the lumen, the second opening, the tunnel, and the first opening. The hollow needle is configured to enter a sampling chamber within an ingestible device to allow at least a portion of a sample in the ingestible device to pass through the fluid channel.

The first surface can be a top surface, and the second surface can be a bottom surface.

The adapter device can further include a removable tube having a tube opening facing the first opening when the removable tube is connected to the top surface.

The removable tube can be connected to the top surface by at least one magnet.

The adapter device can further include: a second tunnel within the body which connects a third opening in the first surface to a fourth opening in the second surface; and a second hollow needle protruding from the second surface and having a second lumen that connects to the fourth opening to define a second fluid channel from the second lumen, the fourth opening, the second tunnel, and the third opening.

The second hollow needle can be configured to enter a second sampling chamber within the ingestible device to allow a second portion of the sample to pass through the second fluid channel.

The second hollow needle can be configured to enter the sampling chamber within the ingestible device simultaneously with the first hollow needle so that the first fluid channel and the second fluid channel define a single fluid channel.

The adapter device can be configured to result in less than 5% sample contamination.

In one aspect, the disclosure provides a grating device configured to remove a portion of a housing of an ingestible device to expose a sampling chamber containing a sample within the ingestible device. The grating device includes: a holder comprising a rail and a recessed area sized and configured to snugly hold a first portion of an ingestible device while exposing a second portion of the ingestible device; a slide block configured to move along the rail and pass over the second portion of the ingestible device; and a grating surface on the slide block and positioned so that when the slide block passes over the second portion of the ingestible device, the grating surface scrapes against the second portion of the ingestible device to remove a portion of the housing of the ingestible device and expose the sampling chamber.

The second surface can a top surface.

The second surface can be a bottom surface.

The grating surface can be connected to the slide block by one or more magnetic plates.

The grating surface can be connected to the slide block by one or more mechanical fasteners.

The grating surface can include at least one member selected from the group consisting of a blade, a roughened surface, and sand paper.

A spring can be positioned between the grating surface and a surface of the slide block, and the spring can push the grating surface towards the ingestible device when the slide block passes over the second portion of the ingestible device.

The grating surface can be a first grating surface, and the grating device further include a second grating surface on the slide block and positioned so that, when the slide block passes over the second portion of the ingestible device, the second grating surface scrapes against the second portion of the ingestible device to remove a second portion of the housing of the ingestible device.

The first grating surface can be perpendicular to the second grating surface.

The holder can include a first set of orientation markings proximate to the recessed area to indicate the removed portion of the housing of the ingestible device when the slide block passes over the second portion of the ingestible device.

The slide block can include a groove-shaped slot extending along a length of the slide block, and the groove-shaped slot can be configured to fit at least partially around the rail as the slide block moves along the rail.

The rail can be a first rail, the holder can include a second rail positioned substantially on an opposite side of the holder from the first rail, and the slide block can be configured to move along the first rail and the second rail simultaneously.

The groove-shaped slot can include a first groove-shaped slot, the slide block can have a second groove-shaped slot extending along the length of the slide block, and the second groove-shaped slot can be positioned substantially on an opposite side of the slide block from the first groove-shaped slot and shaped to fit at least partially around the second rail as the slide block moves along the second rail.

In one aspect, the disclosure provides a method that includes removing a sample from an ingestible device so that there is less than 5% sample contamination. The can be less than 4% (e.g., less than 3%, less than 2%, less than 1%, 0%) sample contamination.

The method can include removing at least two different samples from the ingestible device, wherein there is less than 5% (e.g., less than 4%, less than 3%, less than 2%, less than 1%, 0%) contamination of either sample.

The method can include using centrifugation to remove the sample from the ingestible device.

Prior to removing the sample, the sample can be contained in a sampling chamber within the ingestible device.

Methods can further include, prior to centrifuging, determining a location of the ingestible device in a portion of a GI tract of a subject to an accuracy of at least 75%.

Methods can further include, after determining the location of the ingestible device, obtaining the sample.

An ingestible device can include: one or more processing devices; and one more machine readable hardware storage devices storing instructions that are executable by the one or more processing devices to transmit data to a device capable of implementing the data to determine a location of the medical device in a portion of a GI tract of a subject to an accuracy of at least 85%.

An ingestible device can include: one or more processing devices; and one more machine readable hardware storage devices storing instructions that are executable by the one or more processing devices to determine a location of the ingestible device in a portion of a GI tract of a subject to an accuracy of at least 85%.

In some aspects, a method for extracting a sample from an ingestible device, the ingestible device having a housing, is provided. The method includes causing an opening to be created in the housing of the ingestible device, the opening connected to a sampling chamber within the ingestible device; inserting the ingestible device at least partially into a fitted sleeve, the fitted sleeve connecting the opening in the housing of the ingestible device to a tube; and centrifuging the ingestible device, while the ingestible device is at least partially inserted into the fitted sleeve, to transfer at least a portion of the sample contained in the sampling chamber into the tube.

In at least some embodiments, causing the opening to be created includes inserting at least a portion of the ingestible device into a lancing jig having a lancet; causing the lancet to heat to a temperature within a predetermined temperature range; and causing the heated lancet to puncture the housing of the ingestible device, thereby creating the opening.

In at least some embodiments, each temperature in the predetermined temperature range is above a melting point of a material forming the housing of the ingestible device.

In at least some embodiments, causing the opening to be created includes inserting at least a portion of the ingestible device into a recessed area of a holder; and sliding a grating surface on a moveable slide block against the ingestible device to remove a portion of the housing of the ingestible device and create the opening.

In at least some embodiments, the method further includes causing a second opening to be created in the housing of the ingestible device, the second opening being connected to a second sampling chamber within the ingestible device.

In at least some embodiments, a second portion of the sample is contained in the second sampling chamber before the centrifuging, and the centrifuging transfers the second portion of the sample from the ingestible device into a second tube.

In at least some embodiments, the portion of the sample is absorbed by a sponge within the sampling chamber prior to being transferred into the tube.

In at least some embodiments, the method includes disconnecting the tube from the fitted sleeve after centrifuging the ingestible device.

In at least some embodiments, the centrifuging includes inserting the ingestible device and the fitted sleeve into a centrifuge tube; attaching the centrifuge tube to a cap having a spring on a bottom surface of the cap; causing the spring to maintain a position of the ingestible device relative to the fitted sleeve as the ingestible device is at least partially inserted into the fitted sleeve; and centrifuging the centrifuge tube while the ingestible device and the fitted sleeve are inserted into the centrifuge tube.

In at least some embodiments, the method includes using a moveable screw positioned on a side of the centrifuge tube to restrict movement of the ingestible device relative to the centrifuge tube.

In at least some embodiments, the housing of the ingestible device includes an indentation, and the opening is created at a location of the indentation.

In at least some embodiments, the sample is obtained by the ingestible device from a predetermined region of the gastrointestinal tract.

In some aspects, a sleeve device for use in extracting a sample from an ingestible device having a housing, a first end with a first opening that leads to a sampling chamber containing the sample, and a second end, is provided herein. The sleeve device includes a sleeve portion shaped to fit snugly around a circumference of the ingestible device; and a base portion having a top surface with a top opening, a bottom surface with a bottom opening, and a tunnel connecting the top opening to the bottom opening, wherein the top surface is connectable to an end of the sleeve portion; and the top surface includes a seal portion that is sized and shaped to fit the first end of the ingestible device and form a watertight seal between the top opening and the first opening.

In at least some embodiments, the sleeve device includes a removable tube having a tube opening that faces the bottom opening of the base portion when the removable tube is connected to the bottom surface of the base portion.

In at least some embodiments, the removable tube is connected to the bottom surface of the base portion by one or more magnets.

In at least some embodiments, the sleeve portion and the base portion are connected by a dowel joint.

In at least some embodiments, the sleeve portion and the base portion are connected by a mortise and a tenon joint.

In at least some embodiments, the sleeve device includes a first dowel-shaped opening on the bottom surface of the base portion, a second dowel-shaped opening on the top surface of the base portion, and a first dowel-shaped tunnel connecting the first dowel-shaped opening to the second dowel-shaped opening; a third dowel-shaped opening on the end of the sleeve portion, the third dowel-shaped opening connecting to a second dowel-shaped tunnel extending into the sleeve portion; and a dowel within the first dowel shaped opening, the first dowel shaped tunnel, the second dowel shaped opening, the third dowel shaped opening, and the second dowel shaped tunnel.

In at least some embodiments, the sleeve device includes a window located on a side of the sleeve portion, the window usable to align the ingestible device to the sleeve portion such that the top opening faces the first opening.

In at least some embodiments, the sleeve device includes an aperture located on a side of the sleeve portion; and a positioning pin sized and shaped to be inserted into the aperture, such that inserting the positioning pin into the aperture fixes the orientation of the ingestible device relative to the sleeve portion.

In at least some embodiments, the base portion includes a second tunnel connecting a second top opening on the top surface of the base portion to a second bottom opening on the bottom surface of the base portion; and a second seal portion that is sized and shaped to fit on the first end of the ingestible device and form a second watertight seal between the second top opening and a second opening on the first end of the ingestible device.

In at least some embodiments, the sleeve device includes a second removable tube having a second tube opening that faces the second bottom opening of the base portion when the second removable tube is connected to the bottom surface of the base portion.

In some aspects, another method for extracting a sample from within an ingestible device, the ingestible device having a housing, is provided herein. The method includes separating the ingestible device into a first portion and a second portion, the first portion comprising a sampling chamber that contains at least a portion of the sample; connecting the sampling chamber to an adapter that connects to a tube; and centrifuging the first portion of the ingestible device, while the sampling chamber is connected to the adapter, to transfer at least the portion of the sample from the sampling chamber into the tube.

In at least some embodiments, separating the ingestible device into the first portion and the second portion includes inserting the ingestible device into a cutting device having a recessed area and a moveable blade such that the ingestible device is positioned within the recessed area; and causing the moveable blade to cut the ingestible device to form the first portion and the second portion while the recessed area holds the ingestible device.

In at least some embodiments, separating the ingestible device into the first portion and the second portion exposes a barrier within the first portion of the ingestible device, the barrier forming a portion of an outer surface of the sampling chamber.

In at least some embodiments, connecting the first portion of the ingestible device to the adapter includes inserting the adapter into the first portion of the ingestible device, such that a hollow needle on the adapter penetrates the barrier and enters the sampling chamber.

In at least some embodiments, the hollow needle penetrates an indentation on the barrier.

In at least some embodiments, the centrifuging transfers at least a second portion of the sample from the ingestible device into a second tube, the second portion of the sample contained in a second sampling chamber prior to being transferred into the second tube.

In at least some embodiments, the portion of the sample is absorbed by a sponge within the sampling chamber prior to being transferred into the tube.

In at least some embodiments, the method includes disconnecting the tube from the adapter after centrifuging the first portion of the ingestible device.

In at least some embodiments, the centrifuging includes inserting the first portion of the ingestible device and the adapter into a centrifuge tube; attaching the centrifuge tube to a cap having a spring on a bottom surface of the cap; causing the spring to maintain a position of the first portion of the ingestible device relative to the adapter; and

centrifuging the centrifuge tube while the first portion of the ingestible device and the adapter are inserted into the centrifuge tube.

In at least some embodiments, the method includes using a moveable screw positioned on a side of the centrifuge tube to restrict movement of the first portion of the ingestible device relative to the centrifuge tube.

In at least some embodiments, the sample is obtained by the ingestible device from a predetermined region of the gastrointestinal tract.

In some aspects, an adapter device for use in extracting a sample from a portion of ingestible device is provided herein. The adapter device includes a top surface having a first opening; a bottom surface having a second opening; a body positioned between the top surface and the bottom surface, wherein the body includes a tunnel connecting the first opening to the second opening; and a hollow needle protruding from the bottom surface and having a lumen that connects to the second opening, such that a fluid channel is formed from the lumen, the second opening, the tunnel, and the first opening, wherein the hollow needle is sized and shaped to enter a sampling chamber within the ingestible device to allow at least a portion of the sample to pass through the fluid channel.

In at least some embodiments, the adapter device includes a removable tube having a tube opening that faces the first opening when the removable tube is connected to the top surface.

In at least some embodiments, the removable tube is connected to the top surface by at least one magnet.

In at least some embodiments, the adapter device includes a second tunnel within the body that connects a third opening in the top surface to a fourth opening in the bottom surface; and a second hollow needle protruding from the bottom surface and having a second lumen that connects to the fourth opening, such that a second fluid channel is formed from the second lumen, the fourth opening, the second tunnel, and the third opening.

In at least some embodiments, the second hollow needle is sized and shaped to enter a second sampling chamber within the ingestible device to allow a second portion of the sample to pass through the second fluid channel.

In at least some embodiments, the second hollow needle is sized and shaped to enter the sampling chamber within the ingestible device simultaneously with the first hollow needle, such the first fluid channel and the second fluid channel form a single fluid channel.

In some aspects, a grater device for removing a portion of a housing of an ingestible device and exposing a sampling chamber containing a sample within the ingestible device is provided herein. The grater device includes a holder comprising a rail and a recessed area sized and shaped to snugly hold a bottom portion of the ingestible device while exposing a top portion of the ingestible device; a slide block configured to move along the rail and pass over the top portion of the ingestible device; and a grating surface on the slide block, positioned such that when the slide block passes over the top portion of the ingestible device, the grating surface scrapes against the top portion of the ingestible device to remove the portion of the housing of the ingestible device and expose the sampling chamber.

In at least some embodiments, the grating surface is connected to the slide block by one or more magnetic plates.

In at least some embodiments, the grating surface is connected to the slide block by one or more mechanical fasteners.

In at least some embodiments, the grating surface includes at least one of a blade, a roughened surface, and sand paper.

In at least some embodiments, a spring is positioned between the grating surface and a first surface of the slide block, and the spring pushes the grating surface towards the ingestible device when the slide block passes over the top portion of the ingestible device.

In at least some embodiments, the grating surface is a first grating surface, and the grating device includes a second grating surface on the slide block, positioned such that when the slide block passes over the top portion of the ingestible device, the second grating surface scrapes against the top portion of the ingestible device to remove a second portion of the housing of the ingestible device.

In at least some embodiments, the first grating surface and the second grating surface are oriented perpendicularly to each other.

In at least some embodiments, the holder includes a first set of orientation markings proximate to the recessed area that indicate the removed portion of the housing of the ingestible device when the slide block passes over the top portion of the ingestible device.

In at least some embodiments, the slide block has a groove-shaped slot extending along a length of the slide block, the groove-shaped slot shaped to fit at least partially around the rail as the slide block moves along the rail.

In at least some embodiments, the rail is a first rail, the holder includes a second rail positioned substantially on an opposite side of the holder from the first rail, and the slide block is configured to move along the first rail and the second rail simultaneously.

In at least some embodiments, the groove-shaped slot is a first groove-shaped slot, the slide block has a second groove-shaped slot extending along the length of the slide block, and the second groove-shaped slot is positioned substantially on an opposite side of the slide block from the first groove-shaped slot and shaped to fit at least partially around the second rail as the slide block moves along the second rail.

### BRIEF DESCRIPTION OF THE DRAWINGS

FIG. 1 shows an illustrative embodiment of a method for extracting a sample from an ingestible device.

FIG. 2 shows an illustrative embodiment of a method for creating an opening in an ingestible device using a heated lancet, which may be used in conjunction with the method illustrated by FIG. 1.

FIG. 3 shows an illustrative embodiment of a lancing device, which may be used to create an opening in an ingestible device, and may be used in conjunction with the method illustrated by FIG. 1.

FIG. 4 shows an illustrative embodiment of a grating device, which may be used to create an opening in an ingestible device, and may be used in conjunction with the method illustrated by FIG. 1.

FIG. 5 shows an illustrative embodiment of a sleeve device, which may connect the ingestible device to a tube, and may be used in conjunction with the method illustrated by FIG. 1.

FIG. 6 shows an illustrative embodiment of a centrifuge mechanism, which may allow the sleeve device and the ingestible device to be inserted into a centrifuge, and may be used in conjunction with the method illustrated by FIG. 1.

FIG. 7 shows another illustrative embodiment of a centrifuge mechanism, which may allow the sleeve device and the ingestible device to be inserted into a centrifuge, and may be used in conjunction with the method illustrated by FIG. 1.

FIG. 8 shows an illustrative embodiment of a method for separating the ingestible device into multiple portions, and extracting a sample from a portion of the ingestible device.

FIG. 9 shows an illustrative embodiment of an adapter device, which may connect a portion of an ingestible device to a tube, and may be used in conjunction with the method illustrated by FIG. 8.

FIG. 10 illustrates a system for extracting a sample from an ingestible device.

FIG. 11 is a partial exploded view of the system illustrated in FIG. 10.

FIGS. 12A and 12B illustrate views of an ingestible device with holes for allowing sample to exit samples chambers in the device.

FIG. 13 illustrates a cross-sectional view of an ingestible device with holes for allowing sample to exit samples chambers in the device.

FIG. 14 shows a partial view of an ingestible device.

FIGS. 15A-15C show illustrate operation of ingestible device 5010.

FIG. 16 illustrates an exploded view of the components of ingestible device.

FIG. 17 is a view of an example embodiment of an ingestible device, in accordance with some embodiments of the disclosure.

FIG. 18 is an exploded view of the ingestible device of FIG. 17, in accordance with some embodiments of the disclosure.

FIG. 19 is a diagram of an ingestible device during an example transit through a GI tract, in accordance with some embodiments of the disclosure.

FIG. 20 is a diagram of an ingestible device during an example transit through a jejunum, in accordance with some embodiments of the disclosure.

FIG. 21 is a flowchart of illustrative steps for determining a location of an ingestible device as it transits through a GI tract, in accordance with some embodiments of the disclosure.

FIG. 22 is a flowchart of illustrative steps for detecting transitions from a stomach to a duodenum and from a duodenum back to a stomach, which may be used when determining a location of an ingestible device as it transits through a GI tract, in accordance with some embodiments of the disclosure.

FIG. 23 is a plot illustrating data collected during an example operation of an ingestible device, which may be used when determining a location of an ingestible device as it transits through a GI tract, in accordance with some embodiments of the disclosure.

FIG. 24 is another plot illustrating data collected during an example operation of an ingestible device, which may be used when determining a location of an ingestible device as it transits through a GI tract, in accordance with some embodiments of the disclosure.

FIG. 25 is a flowchart of illustrative steps for detecting a transition from a duodenum to a jejunum, which may be used when determining a location of an ingestible device as it transits through a GI tract, in accordance with some embodiments of the disclosure.

FIG. 26 is a plot illustrating data collected during an example operation of an ingestible device, which may be used when detecting a transition from a duodenum to a jejunum, in accordance with some embodiments of the disclosure.

FIG. 27 is a plot illustrating muscle contractions detected by an ingestible device over time, which may be used when determining a location of an ingestible device as it transits through a GI tract, in accordance with some embodiments of the disclosure.

FIG. 28 is a flowchart of illustrative steps for detecting a transition from a jejenum to an ileum, which may be used when determining a location of an ingestible device as it transits through a GI tract, in accordance with some embodiments of the disclosure.

FIG. 29 is a flowchart of illustrative steps for detecting a transition from a jejenum to an ileum, which may be used when determining a location of an ingestible device as it transits through a GI tract, in accordance with some embodiments of the disclosure.

FIG. 30 is a flowchart of illustrative steps for detecting a transition from an ileum to a cecum, which may be used when determining a location of an ingestible device as it transits through a GI tract, in accordance with some embodiments of the disclosure.

FIG. 31 is a flowchart of illustrative steps for detecting a transition from a cecum to a colon, which may be used when determining a location of an ingestible device as it transits through a GI tract, in accordance with some embodiments of the disclosure.

### DETAILED DESCRIPTION

To provide an overall understanding of the disclosure, certain illustrative embodiments will now be described, including various systems and methods for extracting samples from ingestible devices, or for moving substances into or within ingestible devices. In particular, techniques are described that allow samples to be extracted from an ingestible device, after the samples have been obtained from within a gastrointestinal (GI) tract. These samples may include any of the fluids, solids, particulates, or other substances found within the GI tract. However, it will be understood by one of ordinary skill in the art that the systems and methods described herein may be adapted and modified as is appropriate for the applications being addressed, and that the systems and methods described herein may be employed in other suitable applications, and that such other additions and modifications will not depart from the scope of the present disclosure. Generally, the systems and methods described herein may be incorporated into other systems and methods, and portions of the systems and methods described herein may be partially or fully automated through the use of appropriate actuators, sensors, valves, chambers, logic devices, microcontrollers, or other devices and processors that may be configured using a combination of hardware, firmware, and software.

It should be noted that terms of degree such as “substantially”, “about” and “approximately” when used herein mean a reasonable amount of deviation of the modified term such that the end result is not significantly changed. These terms of degree should be construed as including a deviation of the modified term if this deviation would not negate the meaning of the term it modifies.

In addition, as used herein, the wording “and/or” is intended to represent an inclusive “or”. That is, “X and/or Y” is intended to mean X or Y or both, for example. As a further example, “X, Y, and/or Z” is intended to mean X or Y or Z or any combination thereof.

As used herein, the term “coupled” indicates that two elements can be directly coupled to one another or coupled to one another through one or more intermediate elements.

As used herein, the term “body” refers to the body of a patient, a subject or an individual who receives the ingestible device. The patient or subject is generally a human or other animal.

As used herein, the term “gastrointestinal tract” or “GI tract” refers to all portions of an organ system responsible for consuming and digesting foodstuffs, absorbing nutrients, and expelling waste. This includes orifices and organs such as the mouth, throat, esophagus, stomach, small intestine, large intestine, rectum, anus, and the like, as well as the various passageways and sphincters connecting the aforementioned parts.

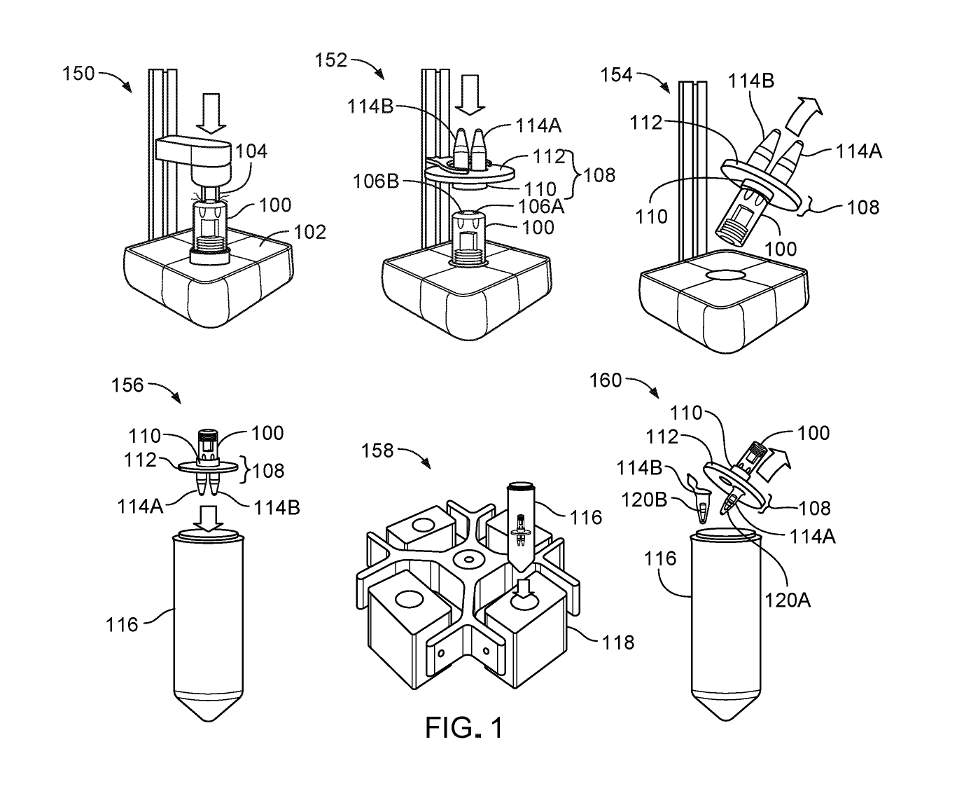

For illustrative purposes, the ingestible devices depicted in FIGS. 1-9 are shaped like a capsule, with a housing that has a first end, a second end, and a longitudinal wall connecting the first end to the second end. The ingestible devices depicted by FIGS. 1-9 may have sampling chambers located at the first end of the device, within the housing of the ingestible device. Examples of these types of ingestible devices are discussed in greater detail in PCT Application No. PCT/CA2013/000133 filed Feb. 15, 2013, which is hereby incorporated by reference herein in its entirety. However, the methods and systems described herein are not limited to these types of devices, and it will be understood that the systems and methods described herein may be easily modified to accommodate any type of ingestible device, regardless of how the sample is obtained, the shape of the ingestible device, or the location of the sampling chamber within the ingestible device. Examples of other types of ingestible devices, which may have sampling chambers located in other positions within the ingestible device, are discussed in greater detail in U.S. Provisional Application No. 62/376,688 filed Aug. 18, 2016, which is hereby incorporated by reference herein in its entirety.

For illustrative purposes, the ingestible devices depicted in FIGS. 1-9 may have multiple openings, or multiple sampling chambers. In general, it is possible for the ingestible device to obtain different samples from different predetermined regions of the GI tract. For example, it may be possible for an ingestible device to use various combinations of light emitting diodes and sensors to determine whether the device is in the stomach, small intestine, or large intestine. This may be done by emitting light at different wavelengths, measuring the level of light reflected at each wavelength by the environment surrounding the ingestible device, and using this information to determine an approximate location of the ingestible device based on the different reflectance properties of the various different portions of the GI tract. Once the ingestible device determines that it is in a particular predetermined portion of the GI tract (e.g., the small intestine), the ingestible device may be configured to obtain a sample from that portion of the GI tract, and store the sample in one or more sampling chambers within the ingestible device. Additional examples of systems and methods for determining the location of an ingestible device within a GI tract and obtaining samples from predetermined portions of the GI tract are discussed in greater detail in PCT Application No. PCT/US2015/052500 filed on Sep. 25, 2015, which is hereby incorporated by reference herein in its entirety. Although one of the many advantages of the systems and methods discussed in relation to FIGS. 1-9 may include an ability to efficiently retrieve multiple samples from multiple sampling chambers within an ingestible device while reducing the risk of cross-contamination between samples, the systems and methods discussed in relation to FIGS. 1-9 may be adapted to extract samples from any number of sampling chambers, including a single sampling chamber.

FIG. 1 shows an illustrative embodiment of a method for extracting a sample from an ingestible device. The method includes causing an opening to be created in the housing of the ingestible device. An example of creating an opening is shown in diagram 150. The method includes inserting the ingestible device at least partially into a fitted sleeve that connects the opening in the housing of the ingestible device to a tube. An example of this process is shown in diagrams 152 and 154. The method also includes centrifuging the ingestible device, while the ingestible device is at least partially inserted into the fitted sleeve, to transfer at least a portion of the sample contained in the sampling chamber into the tube. An example of a centrifuging process is shown in diagrams 156, 158, and 160.

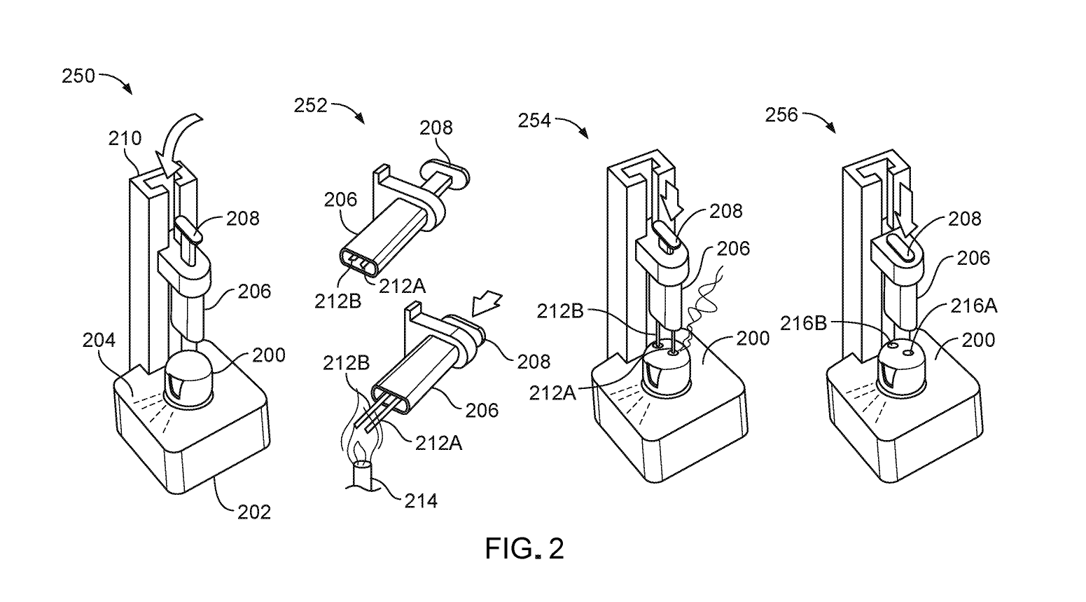

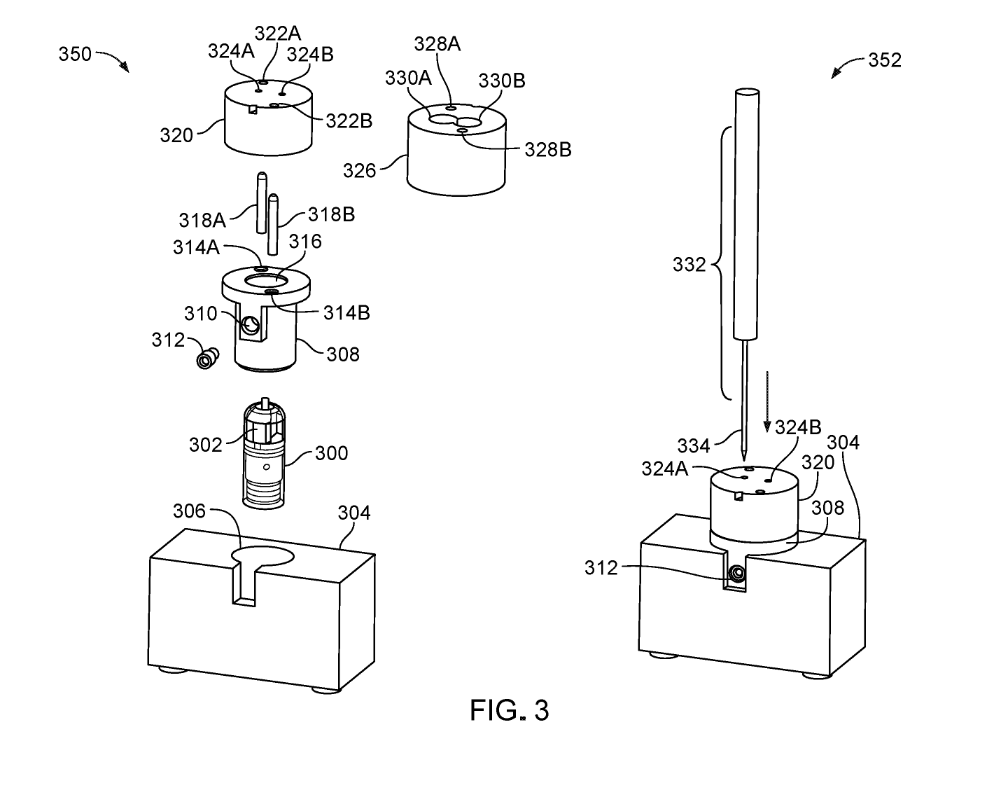

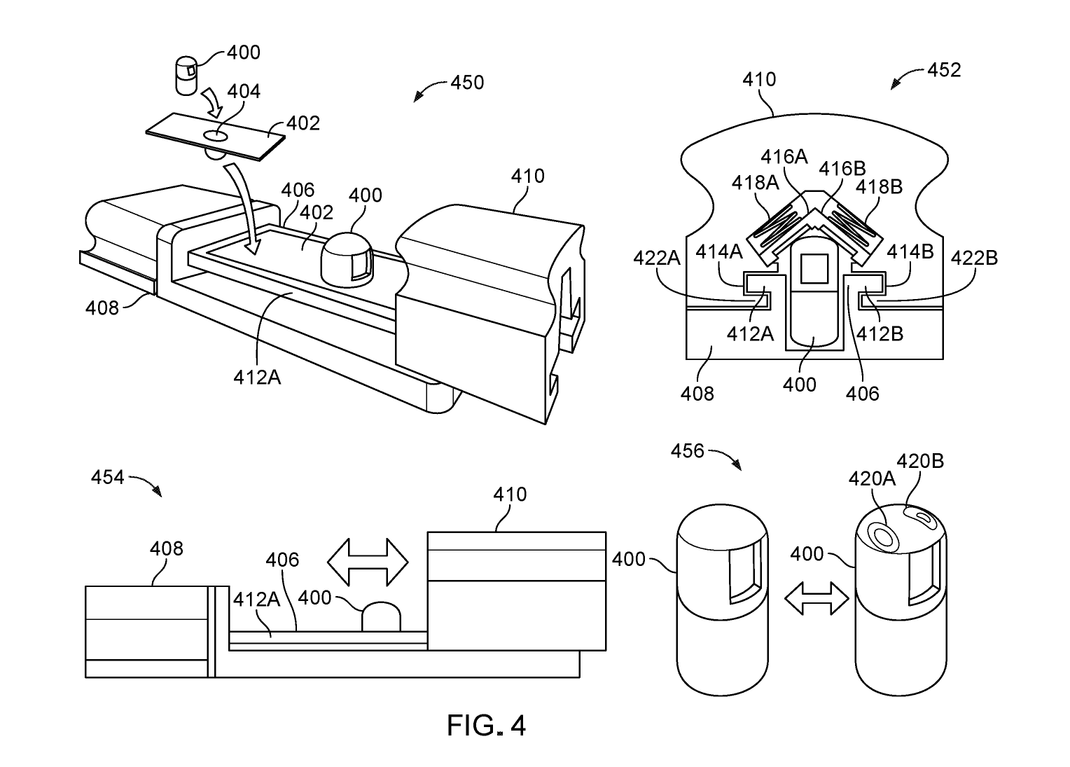

As shown in diagram 150, an opening is created in the housing of the ingestible device 100. The ingestible device 100 is placed into a holder 102, and a lancing mechanism 104 including two lancets is used to pierce the housing of the ingestible device and create two openings. Other examples of methods and systems for creating an opening in an ingestible device are discussed in relation to FIG. 2, FIG. 3, and FIG. 4.

In general, ingestible devices may contain one or more sampling chambers, and each of the created openings may connect directly to one or more of the sampling chambers. In the example shown in diagram 150, the ingestible device 100 has sampling chambers located near the top of the device, which is where the two openings are created by the lancing mechanism 104. However, it is possible for any number of openings to be connected to a single sampling chamber, and it may also be possible for each opening to be connected to different sampling chambers.

The lancing mechanism 104 is depicted in diagram 150 as having two lancets and creates two openings in the housing of the ingestible device 100. In general, the lancing mechanism 104 may include any number of lancets, where each lancet may be used to create an opening in the housing. Moreover, the lancing mechanism 104 may include one or more lancets that are not used to create an opening in one ingestible device, but are used to create one or more openings in another, larger ingestible device with a larger number of sampling chambers, for example.

As shown in diagram 152, the ingestible device 100 is at least partially inserted into a sleeve device 108. After the openings 106A and 106B (generally, openings 106) are created in the ingestible device 100, the sleeve device 108 is connected to the ingestible device 100. The sleeve device 108 includes a sleeve portion 110, and a base portion 112. The sleeve portion 110 may be sized and shaped to fit snugly around a portion of the ingestible device, and the base portion 112 may be sized and shaped to connect the openings 106A and 106B to the tubes 114A and 114B respectively.

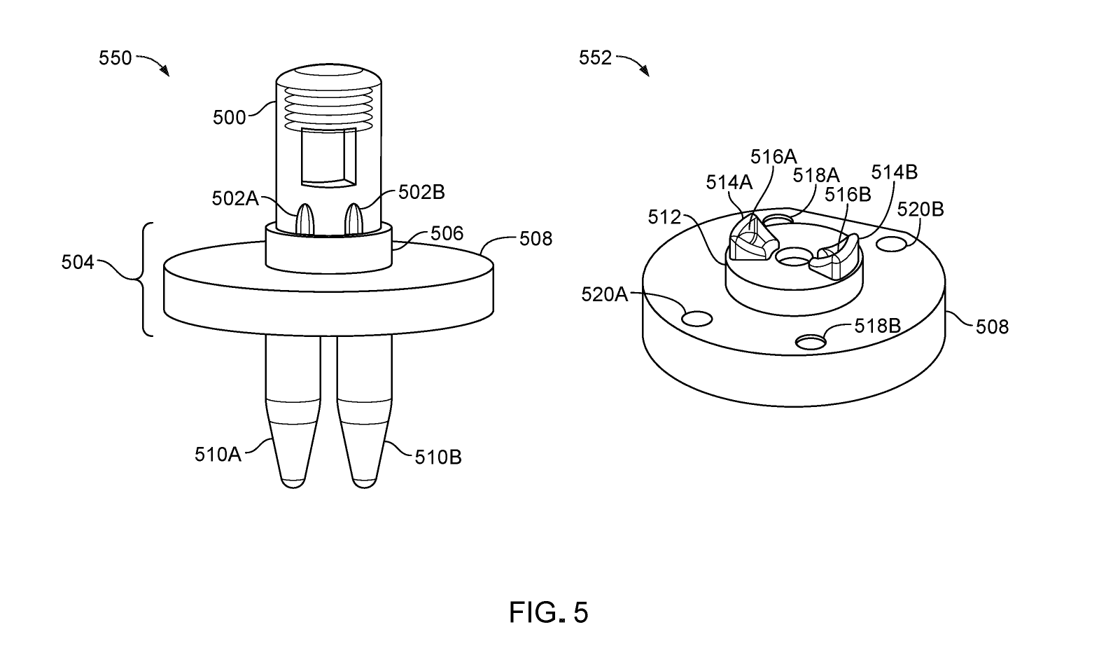

Diagram 154 shows removing the ingestible device 100 from the holder 102 after the ingestible device 100 is connected to the sleeve device 108. In general, the sleeve portion 110 of the sleeve device 108 may fit snugly around the ingestible device 100, and form a seal around a portion of the ingestible device 100. The base portion 112 of the sleeve device 108 may form a seal around each of the openings in the ingestible device 100, connecting each of the openings directly to one of the tubes 114A or 114B. An example of a sleeve device 108, and how the connection to the tubes 114A and 114B may be created, is discussed in detail in relation to FIG. 5.

As shown in diagrams 156, 158, and 160, the ingestible device is centrifuged. The centrifuging occurs while the ingestible device is at least partially inserted into the fitted sleeve and is performed in order to transfer at least a portion of the sample contained in the sampling chamber into the tube. Diagram 156 shows the ingestible device 100 being inserted into a centrifuge tube 116 while the ingestible device 100 is connected to the tubes 114A and 114B via the sleeve device 108. Generally, the ingestible device 100 and the sleeve device 108 may be placed within the centrifuge tube 116. The tubes 114A and 114B may be oriented near the bottom of the centrifuge tube 116, and the ingestible device 100 may be oriented near the top of the centrifuge tube 116. This may ensure that centrifugal forces transfer a portion of the sample contained within the ingestible device 100 into the tubes 114A and 114B.

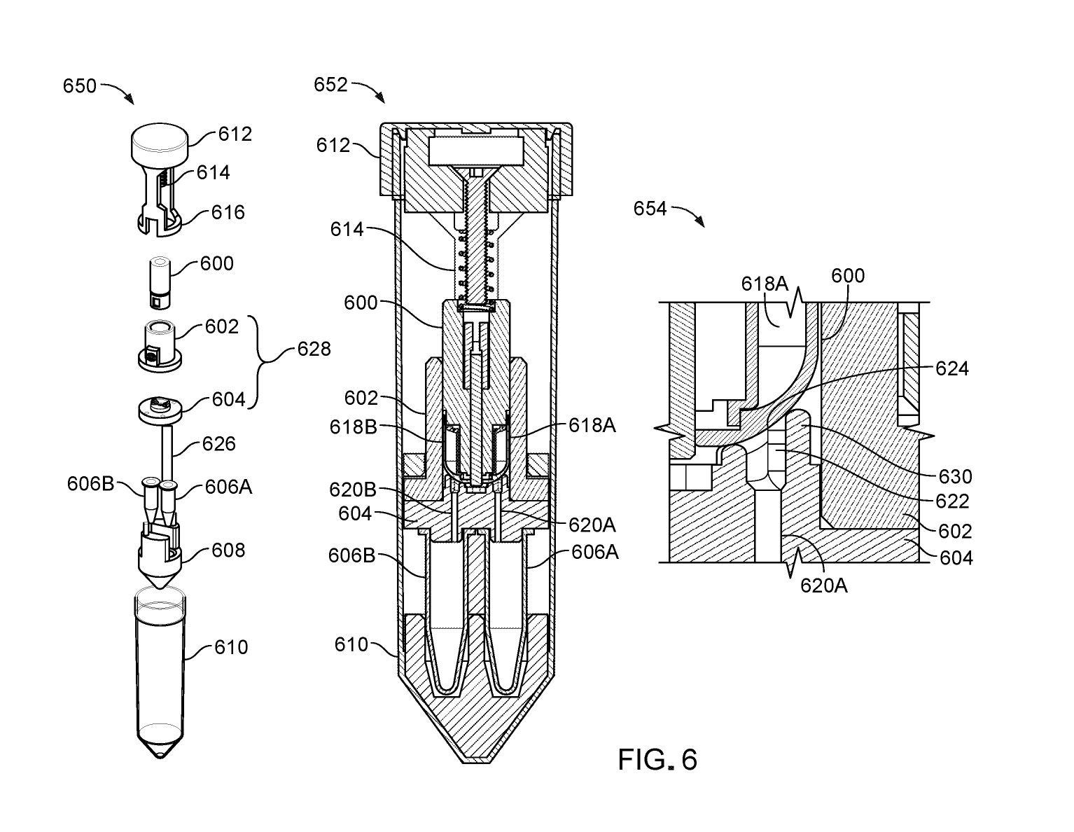

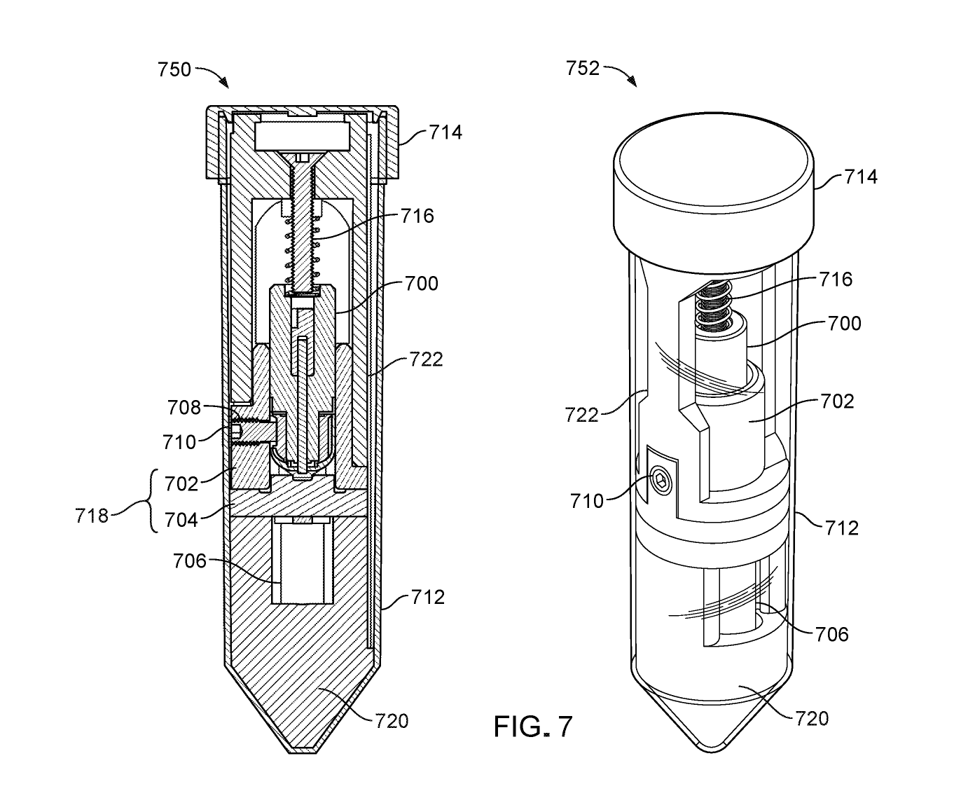

The centrifuge tube 116 may be a commercially available centrifuge tube, such as a Falcon™ brand 50 mL conical centrifuge tube. However, in some embodiments, the centrifuge tube 116 is modified in order to better secure the ingestible device 100 and the sleeve device 108 within the centrifuge tube 116. Examples of optional centrifuge mechanisms, which may allow the sleeve device 108 and the ingestible device 100 to be secured within the centrifuge tube 116, are discussed in relation to FIG. 6 and FIG. 7.

Diagram 158 shows the centrifuge tube 116 (which contains the ingestible device 100, the sleeve device 108, and the tubes 114A and 114B) being inserted into a centrifuge 118. After the centrifuge tube 116 is inserted into the centrifuge 118, the centrifuge 118 may be turned on, thereby centrifuging the ingestible device 100. It will be understood that the speed and length of time that the ingestible device 100 is centrifuged may depend on any number of factors, such as the physical properties of the ingestible device 100, the properties of the sample contained within the ingestible device 100, and the amount of the sample to be extracted from within the ingestible device 100. However, for typical applications involving an ingestible device of the type described in PCT Application No. PCT/CA2013/000133 containing a sample acquired from the small intestine, centrifuging the ingestible device for 4 minutes at 3700 rpm is sufficient to transfer a portion of the sample from the ingestible device 100 into the tubes 114A and 114B.

Diagram 160 shows the ingestible device 100, the sleeve device 108, and the tubes 114A and 114B being removed from the centrifuge tube 116, after the ingestible device 100 and the sleeve device 108 have been centrifuged. The centrifugal forces during the centrifuge process described in relation to diagram 158 have transferred portions of the sample 120A and 120B (generally, portions of the sample 120) from the ingestible device 100 into the tubes 114A and 114B. The tubes 114A and 114B containing the samples 120A and 120B may then be disconnected from the sleeve device 108. The tubes 114A and 114B containing the samples 120A and 120B may then be used in any type of standard laboratory diagnostics, and the ingestible device 100 and the sleeve device 108 may be disposed of, recycled and sanitized, or repurposed for another use.

In some embodiments, as a result of the centrifuging, tubes 114A and 114B may contain portions of samples 120A and 120B that were contained in separate sampling chambers within the ingestible device 100. This may be the case if opening 106A connects to a first sampling chamber, opening 106B connects to a second sampling chamber, and if each sampling chamber holds a separate portion of the sample 120A and 120B prior to the centrifuging. In some embodiments, the portions of the sample 120A and 120B may be absorbed by a sponge contained within the sampling chambers prior to be being transferred into the tube. For example, the ingestible device 100 may use a sponge within each of the sampling chambers in order to better acquire samples from the ingestible device while the ingestible device 100 traverses the GI tract. In these situations, the force of the centrifuging may extract a portion of the sample from the sponge before the sample is transferred into the tubes 114A and 114B.

The ingestible device 100 may have sampling chambers located near the top of the device, and these sampling chambers are connected to the created openings 106A and 106B. However, some ingestible devices may have sampling chambers located in other positions within the ingestible device. In some embodiments, the positioning and placement of an ingestible device within the holder 102 is altered such that the opening created by the lancing mechanism 104 still connects to the sampling chamber of the ingestible device. Similarly, the general shape of the sleeve device 108 may be altered to fit around any portion of an ingestible device, and to accommodate one or more openings made at any location on the housing of the ingestible device.

In some embodiments, the sleeve device 108 creates the openings in the ingestible device 100. For example, two needles may be attached to the bottom side of the sleeve device 108. As the sleeve device 108 is connected to the ingestible device 100, these needles may pierce the housing of the ingestible device 100, thereby creating the openings 106A and 106B. This may allow openings to be created in the housing of the ingestible device 100 without the use of a lancing mechanism 104.

In some embodiments, a modified version of the sleeve device 108 connects multiple openings 106A and 106B to a single tube 114. For example, the ingestible device 100 may have multiple sampling chambers, each of which is connected to a different one of the created openings 106A and 106B. The sleeve device 108 may connect both of these openings 106A and 106B to a single tube 114. This may allow the substances contained in each of the sampling chambers to be mixed together within the tube 114 as portions of the samples are transferred into the tube 114.

In the examples shown in diagrams 150, 152, 154, 156, 158, and 160, two openings 106A and 106B are simultaneously created in the housing of the ingestible device 100, and the sleeve device 108 is designed to simultaneously connect two tubes 114A and 114B to the ingestible device 100. However, in some embodiments, only a singe opening is created in the housing of the ingestible device 100, and the sleeve device 108 is designed to connect only a single tube to the ingestible device 100. More generally, in some embodiments, any number of openings may be created in the ingestible device 100, either sequentially or simultaneously, and the sleeve device 108 is designed to connect any number of tubes to any number of openings. In some embodiments, the sleeve device 108 may also be designed to connect multiple ingestible devices to any number of tubes.

In the example shown in diagrams 150 and 152, the two openings 106A and 106B are simultaneously created by a lancing mechanism 104. However, in some embodiments, other types of mechanisms may be used to create any number of openings in the ingestible device 100, either sequentially or simultaneously.

In some embodiments, the sleeve device 108 and the tubes 114A and 114B may not be involved in the sample extraction process, and the ingestible device 100 may be loaded directly into the centrifuge tube 116 after the one or more openings are created in the ingestible device 100. This may result in a portion of the sample being transferred directly into the centrifuge tube 116, and the centrifuge tube 116 containing the portion of the sample may then be used for diagnostics and laboratory testing. In some of these embodiments, a first opening is created to access a first sampling chamber in the ingestible device 100, and a first sample is transferred into the centrifuge tube 116 from the first sampling chamber. Afterwards, a second opening is created to access a second sampling chamber in the ingestible device 100, and a second sample is transferred into a different centrifuge tube 116 from the second sampling chamber. This process may be continued for additional samples in additional sampling chambers, and may allow portions of multiple different samples to be transferred into separate centrifuge tubes without the need for the sleeve device 108.

In general, the systems and methods and systems discussed in relation to FIG. 1 may be adapted or combined with other systems and methods. For example, the sleeve device 108, the centrifuge tube 116, and the centrifuge 118 may be used to load substances into an ingestible device. An ingestible device may have one or more openings, and a modified sleeve device 108 may connect the openings to tubes or funnels containing substances to be loaded into the ingestible device, such as a medicament to be delivered to a certain portion of the gastrointestinal tract. The ingestible device and the modified sleeve device 108 may be placed into the centrifuge tube 116, with the ingestible device oriented beneath the modified sleeve device 108, closer to the bottom of the centrifuge tube 116. In this arrangement, the centrifugal forces generated by the centrifuge 118 may force the substances through the modified sleeve device 108 and into the ingestible device 100. After the substances have been loaded into the ingestible device, the openings in the ingestible device may be sealed, and the ingestible device may be administered to a patient. In some embodiments, the material used to seal the openings may be made of a different material than the rest of the housing of the ingestible device.

In some embodiments, a modified version of the sleeve device 108 may allow substances to be loaded into an ingestible device without the use of the centrifuge tube 116 or the centrifuge 118. For example, instead of the tubes 114A and 114B, the sleeve device 108 may allow one or more openings in an ingestible device to be connected to a needle or syringe. The needle or syringe may then be used to load the substances directly into the appropriate location within the ingestible device. The openings in the ingestible device may then be resealed prior to the ingestible device being administered to a patient.

As an alternate example, the centrifuge tube 116 and the centrifuge 118 may be used to move substances within an ingestible device, or mix substances previously contained within multiple chambers of an ingestible device. For example, there may be multiple chambers within the ingestible device, each chamber containing a different substance, and the multiple chambers may be connected together by one or more low-pressure check-valves. Loading the ingestible device into the centrifuge tube 116, and centrifuging the ingestible device using the centrifuge 118, may cause one or more of the low-pressure check-valves to break, thereby allowing the different substances to mix together within the ingestible device. This may allow the ingestible device to be used in a wider variety of applications. For example, a powdered medication may be activated when it is mixed with a solvent or reagent. The medication and the solvent may be pre-loaded into separate chambers of the ingestible device, and a centrifuge may be used to mix the substances together and activate the medication only when the ingestible device is about to be administered to a patient.

In general, this type of mixing may also be performed as substances are loaded into an ingestible device, with or without the use of a centrifuge 118 or check-valves. For example, multiple openings in the housing of an ingestible device may connect to a single chamber within the ingestible device. A modified version of the sleeve device 108 may be used to load multiple different substances into the ingestible device through each of the different openings. This may allow the substances to mix together once they are inside the ingestible device, and prevent the need for substances to be mixed together outside of the ingestible device.

As an alternate example, after the openings 106A and 106B are created in the housing of the ingestible device 100, the sleeve device 108 may connect to a vacuum mechanism instead of tubes 114A and 114B. In this case, the sample is directly sucked from the ingestible device 100 and deposited into a tube or other receptacle attached to the vacuum mechanism. This may be done instead of centrifuging the ingestible device 100, or it may be done either before or after a portion of the sample has been extracted by centrifuging. To assist in this process, multiple openings may be created in the housing of the ingestible device 100 that connect to a single sampling chamber within the ingestible device 100. A modified version of the sleeve device 108 may allow one of these openings to be connected to a vacuum mechanism, and allow another of these openings to be connected to a source of air or flushing fluid. This may allow air or flushing fluid to enter the sampling chamber as the sample is being removed from the sampling chamber. This may prevent pressure from building up within the sampling chamber as the sample is sucked out by the vacuum mechanism, and allow the sample to be sucked out of the sampling chamber more easily.

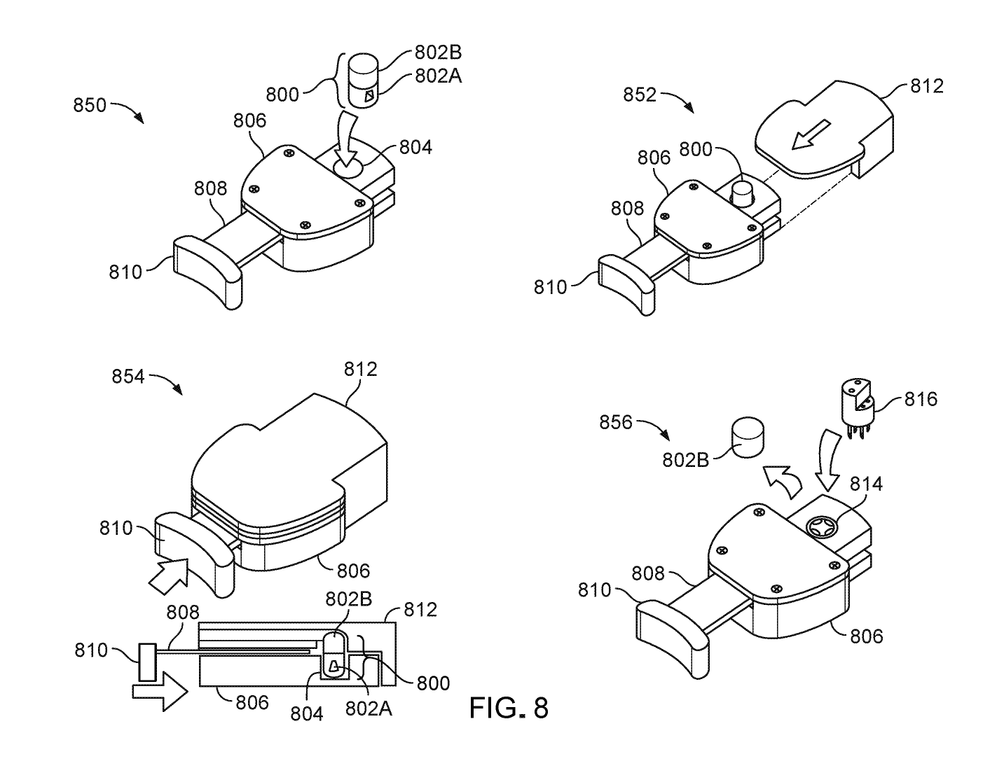

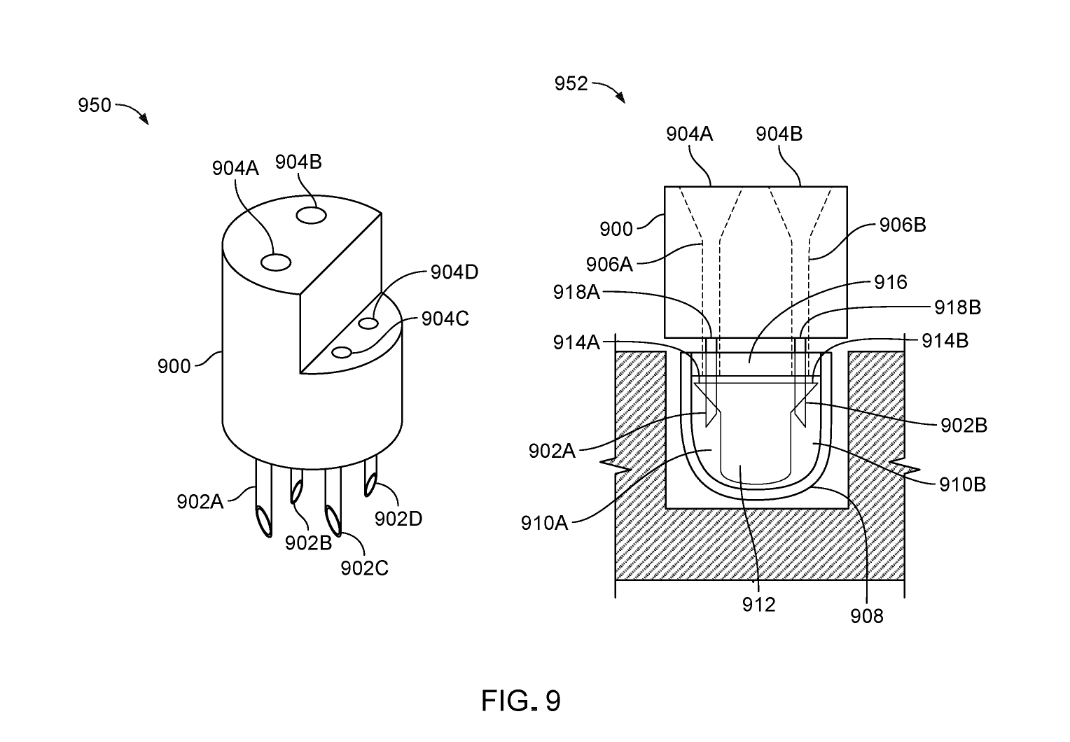

As yet another example, the systems and methods discussed in relation to FIG. 1 may be adapted or combined with any of the systems and methods discussed in relation to FIGS. 2-9. For example, FIG. 8 and FIG. 9 show an illustrative embodiment of a method for separating the ingestible device into multiple portions, and connecting a portion of the ingestible device to an adapter. This adapter may function in a similar manner as the sleeve device 108, and connect the sampling chambers within a portion of an ingestible device 100 to one or more tubes 114. The centrifuge tube 116 and the centrifuge 118 may then be used to extract a sample from the portion of an ingestible device, similar to the manner shown in diagrams 156, 158, and 160.

It is also understood that any of the systems and methods discussed in relation to FIG. 1 may be adapted into automated systems or combined with automated systems. For example, after an ingestible device 100 is inserted into the holder 102, a button or other suitable triggering mechanism may be used to automatically lower the lancing mechanism 104 and create the openings 106A and 106B in the ingestible device 100. Other processes, such as attaching the sleeve device 108 onto the ingestible device 100, or loading the ingestible device 100 and the sleeve device 108 into the centrifuge tube 116, may be similarly automated. In general, the entire process of extracting a sample from an ingestible device described in relation to FIG. 1 may, if desired, be automated.

FIG. 2 shows an illustrative embodiment of a method for creating an opening in an ingestible device using a heated lancet, which may be used in conjunction with the method illustrated by FIG. 1, or which may be combined either wholly or in part with any of the systems and methods discussed in relation to FIGS. 3-9.

Diagram 250 shows the ingestible device 200 placed in a holder 202 of a lancing jig in preparation for two openings to be created in the ingestible device 200 by the lancing mechanism 206. Any one or more characteristics of the ingestible device 100, the holder 102, and the lancing mechanism 104 described in relation to FIG. 1 may be applicable to the ingestible device 200, the holder 202, and the lancing mechanism 206 described in relation to FIG. 2, respectively.

The holder 202 is depicted in diagram 250 with orientation markings 204, which may allow the ingestible device 200 to be aligned in a particular manner within the holder 202. For example, aligning physical characteristics or markings on the ingestible device 200 with the orientation markings 204 may ensure that the lancing mechanism 206 creates openings in the appropriate locations in the housing of the ingestible device 200. In some embodiments, the ingestible device 200 may only be placed into the holder 202 when the ingestible device 200 has a particular orientation relative to the holder 202. For example, the ingestible device 200 may have a window, or other physical feature, on one side of the ingestible device 200, and the holder 202 may have an opening which is sized and shaped to fit snugly around the ingestible device 200 only when the window is oriented in a particular direction (e.g., oriented towards the orientation markings 204 shown in FIG. 2). In some embodiments, the ingestible device 200 may snap into place when it is inserted into the holder 202 in a predetermined orientation, preventing it from moving further. For example, the ingestible device 200 may include a window that is slightly receded from the housing of the ingestible device 200, and the opening of the holder 202 may include a slight protrusion. When the ingestible device 200 is inserted into the holder 202 with the window oriented in a particular position (e.g., oriented towards the orientation markings 204 shown in FIG. 2), the protrusion aligns with the window and fills the receded area. This may prevent the orientation of the ingestible device 200 from changing further once it has been snapped into place.

After the ingestible device 200 has been positioned into the holder 202, the lancing mechanism 206 may be placed into a guide 210. The guide 210 may restrict the motion of the lancing mechanism 206, and ensure that the openings created by the lancing mechanism 206 are positioned correctly. For example, the guide 210 may only permit the lancing mechanism 206 to be lowered to a predetermined depth, and prevent the lancing mechanism 206 from being lowered further. A plunger 208 on an end of the lancing mechanism 206 may be operated in order to push one or more lancets out of the lancing mechanism 206, exposing them, or retract the lancets back into the lancing mechanism 206. In general, the lancing mechanism 206 may include a protective outer sheath, and the lancets are housed within the sheath when the lancets are retracted into the lancing mechanism 206. Retracting the lancets into the protective outer sheath may prevent injuries due to mishandling the lancing mechanism 206. Fully depressing the plunger 208, or depressing the plunger 208 past a predetermined depth, may also cause any exposed lancets to automatically retract back into the lancing mechanism 206. This may be accomplished by means of a spring mechanism.

Diagram 252 shows the operation of the plunger 208 and the lancets 212A and 212B being heated prior to creating the openings in the ingestible device 200. Allowing the lancets 212A and 212B to be retracted into the lancing mechanism 206 may reduce the chance of injury by mishandling or misusing the lancing mechanism 206. This may also prevent the lancets 212A and 212B from being contaminated by the outside environment.

By partially depressing the plunger 208, the lancets 212A and 212B are pushed out of the lancing mechanism 206. A heat source 214 may be used to heat the lancets 212A and 212B. In general, heating the lancets 212A and 212B may sterilize the lancets 212A and 212B, and may reduce the risk of inadvertently contaminating the samples contained within the ingestible device 200 when the openings are created. In some embodiments, the heat source 214 may also heat the lancets 212A and 212B to a temperature that is above the melting point of a material used to form the housing of the ingestible device 200. This may allow the lancets 212A and 212B to easily melt or penetrate the housing of the ingestible device 200, thereby creating openings in the housing of the ingestible device 200.

In general, the temperature of the lancets 212A and 212B is high enough to allow the lancets 212A and 212B to easily penetrate the housing of the ingestible device 200, but not so high that unwanted damage is caused to the ingestible device 200 or any samples contained within the ingestible device 200. For example, the housing of ingestible device 200 may be partially made of polycarbonate that has a melting temperature of about 147 degrees Celsius, and may begin to flow readily at 155 degrees Celsius. In this case, a Bunsen burner or torch may be used as the heat source 214, and the temperature of the lancets 212A and 212B may be raised to approximately 250-300 degrees Celsius prior to being inserted into the ingestible device 200. Having a temperature which is higher than the melting point of the polycarbonate may allow the openings 216A and 216B to be created quickly, and reduce the amount of time that the lancets 212A and 212B are in contact with the ingestible device 200. In turn, this may reduce the risk of damaging the sample, for example, by denaturing analytes contained in the sample fluid. However, keeping the temperature of the lancets 212A and 212B below 300 degrees Celsius may minimize the chance of inadvertently damaging the sample or burning the polycarbonate used to form the housing of the ingestible device 200. It is understood that the temperature ranges above are provided only as examples, and are not limiting. The optimal temperature of the lancets 212A and 212B depends on numerous factors, including the physical properties of the lancets 212A and 212B, the physical properties of the housing of the ingestible device 200 and the sample within the ingestible device 200, the desired size and shape of the openings 216A and 216B, the geometry and shape of the tips of the lancets 212A and 212B, and any number of other factors.

In some embodiments, the lancets 212A and 212B may be replaced by electrically heated tips. For example, the lancets 212A and 212B may be replaced by soldering iron tips that are heated by means of an electrical current instead of an external heat source 214. Replacing the lancets 212A and 212B with electrically heated tips may allow the temperature to be set and maintained accurately.

Diagram 254 shows the lancing mechanism 206 being used to create the openings in the ingestible device 200. By positioning the lancing mechanism 206 slightly above the ingestible device 200, and partially depressing the plunger 208, the heated lancets 212A and 212B are brought into contact with, and penetrate, the housing of the ingestible device 200.

Diagram 256 shows the openings 216A and 216B (generally, openings 216) created in the housing of the ingestible device 200 by the lancets 212A and 212B, after the plunger 208 has been fully depressed. By fully depressing the plunger 208, the lancets 212A and 212B are automatically retracted back into the lancing mechanism 206. This may be done, for example, by means of a spring mechanism within the lancing mechanism 206. Automatically retracting the lancets 212A and 212B when the plunger 208 is fully depressed may reduce the time that a sample contained within the ingestible device 200 is exposed to the heated lancets 212A and 212B. In some embodiments, the lancets 212A and 212B are automatically retracted back into the lancing mechanism 206 when the plunger 208 is depressed past a predetermined depth, and the plunger 208 does not need to be fully depressed.

In some embodiments, fully depressing the plunger 208, or depressing the plunger 208 past a predetermined depth, does not automatically retract the lancets 212A and 212B back into the lancing mechanism 206. Instead, the lancets 212A and 212B may be removed by lifting the plunger 208, or lifting the entire lancing mechanism 206, away from the ingestible device 200. In some embodiments, the lancets 212A and 212B are automatically retracted a predetermined period of time after the plunger 208 has been depressed.

In some embodiments, the holder 202 does not include orientation markings 204. For example, there may be only a single sampling chamber within the ingestible device 200, and the precise positioning of the openings 216A and 216B may be unimportant. As an alternate example, there may be marking on the ingestible device 200 that may be aligned directly with the lancets 212A and 212B. As an alternate example, the portion of the housing of the ingestible device 200 where the openings are to be created are made with a different material than the surrounding portion of the housing. For example, the openings 216A and 216B may more easily created if only a certain portion of the housing of the ingestible device 200 has a lower melting temperature, or is made with a softer material, than the rest of the housing of the ingestible device 200. As yet another example, the ingestible device 200 may include slight indentations on the surface of the housing, and the lancing mechanism 206 may be aligned with the indentations, and create the openings 216A and 216B at the same location as the indentations.

FIG. 2 depicts two openings being created in the housing of the ingestible device 200 simultaneously. However, the lancing mechanism 206 may be modified to create any number of openings in the ingestible device 200, including a single opening in the ingestible device 200. Additionally, different openings may be made at different times. For example, a first opening may be made at a first location by using a lancing mechanism, the ingestible device 200 may be reoriented within the holder 202, and a second opening may be made in a second location using the same lancing mechanism a second time.

In general, the systems and methods discussed in relation to FIG. 2 may be combined with any other systems and methods, including the systems and methods discussed in relation to FIG. 1 or FIGS. 3-9. For example, the lancing mechanism 104 depicted in FIG. 1 may have heated lancets, similar to the lancets 212A and 212B. As an alternate example, the lancing mechanism discussed in relation to FIG. 3 may be a heated lancet, or may automatically retract after the lancet penetrates the housing of an ingestible device. The general concept of heating a blade or surface to better penetrate or create openings in the housing of an ingestible device may also be directly applicable to the systems and methods described in FIGS. 4 and 8. In general, it is understood the any of the systems and methods discussed in relation to FIG. 2 may be incorporated into an automated system for creating openings within an ingestible device. For example, the process of heating and inserting the lancets 212A and 212B may be controlled by one or more sensors or actuators working in tandem with a computer or microcontroller. This could be accomplished, for example, by replacing the lancets 212A and 212B with electrically heated tips, and using threading within the guide 210 to raise and lower the lancing mechanism 206 with a motor. This may allow the openings 216A and 216B to be created with increased precision, and reduce the time that the heated lancets 212A and 212B are in contact with the ingestible device 200.

FIG. 3 shows an illustrative lancing device, which may be used to create an opening in an ingestible device, and may be used in conjunction with the method illustrated by FIG. 1, or which may be combined either wholly or in part with any of the systems and methods discussed in relation to FIG. 2 or FIGS. 4-9. Diagram 350 shows an exploded view of the lancing device, with different components illustrated in detail. Diagram 352 shows the fully assembled lancing device, which allows a lancet 332 to be inserted into an ingestible device.

The ingestible device 300 may be placed into an opening 316 of a sleeve device 308 in a manner similar to the way the ingestible device 100 is inserted into the sleeve portion 110 of the sleeve device 108 discussed in relation to FIG. 1. The ingestible device 300 may be placed in the sleeve device 308, and the sleeve device 308 may be placed into an opening 306 in the holder 304, which may perform a similar function as holder 102 discussed in relation to FIG. 1.

The sleeve device 308 has an aperture 310, which is located on a protruding portion of the sleeve device 308. The opening 306 of the holder 304 is sized and shaped to accommodate sleeve device 308, including the protruding portion, and may ensure that the sleeve device 308 has a predetermined orientation relative to the holder 304 when the sleeve device 308 and the ingestible device 300 are inserted into the opening 306 on the holder 304.

In order to ensure that the ingestible device 300 is oriented properly relative to the sleeve device 308, the aperture 310 may be used to view the ingestible device 300 while it is within the sleeve device 308. The ingestible device 300 has a physical feature or marking 302 that may be a sample acquisition window located on the side of the ingestible device 300. The ingestible device 300 may by rotated in order to line up the feature or marking 302 with the aperture 310, thereby orienting the ingestible device 300 properly within the sleeve device 308. Once the ingestible device 300 is oriented properly relative to the sleeve device 308 and the holder 304, a fitting screw 312 may be placed into the aperture 310. The fitting screw 312 may secure the ingestible device 300 within the sleeve device 308 and prevent the ingestible device 300 from moving or rotating relative to the sleeve device 308.

The openings 314A and 314B (generally, opening 314) on the sleeve device 308 are sized and shaped to accommodate two dowels 318A and 318B (generally, dowel 318). The dowels 318A and 318B may connect the sleeve device 308 to a lancing guide 320, and ensure that the lancing guide 320 has a particular orientation relative to the sleeve device 308. The bottom surface of the lancing guide 320 has a similar pair of openings (not shown), which may allow the lancing guide 320 to slide smoothly onto the dowels 318A and 318B, and come into contact with the top of the sleeve device 308 when the lancing guide 320 is oriented properly relative to the sleeve device 308.

The top surface of the lancing guide 320 may include two openings 322A and 322B (generally, openings 322). Depending on the length of the dowels 318A and 318B, the dowels 318A and 318B may extend all the way through the lancing guide 320, and extend out of the openings 322A and 322B. This may allow additional guides or additional types of devices to be connected to the top surface of the lancing guide 320 and the sleeve device 308. However, in some embodiments, the length of the dowels 318A and 318B may be shorter, and the dowels 318A and 318B do not extend out of the top surface of the lancing guide 320. In this case, the top surface of the lancing guide 320 may not include the openings 332A and 332B.

Referring now to diagram 352, the openings 324A and 324B (generally, opening 324) in the top surface of the lancing guide 320 are sized and shaped to accommodate a pointed end 334 of a lancet 332. Because the lancing guide 320 may have a particular orientation relative to the sleeve device 308, and the sleeve device 308 may have a particular orientation relative to the ingestible device 300, the opening may be created at a predetermined site on the ingestible device 300 when the pointed end 334 of the lancet 332 is inserted through the opening 324A and into the housing of the ingestible device.

As discussed in relation to FIG. 2, the pointed end 334 of the lancet 332 may be heated prior to being inserted into the opening 324. This may allow the pointed end 334 of the lancet 332 to easily slide through the housing of the ingestible device 300. The pointed end 334 may, for example, be heated until it has a temperature within a predetermined range, which may be high enough to melt a material used to create the housing of the ingestible device 300, but not so hot that it damages the lancing mechanism or the sample contained within the ingestible device 300. After a first opening in the ingestible device 300 has been created by inserting the lancet through the opening 324A and penetrating a first site on the housing of the ingestible device 300, a second opening may be created by inserting a lancet through the opening 324B and penetrating a second site on the housing of the ingestible device 300.

In some embodiments, the lancet 332 may be replaced by a soldering iron, and the lancing guide 320 may be replaced with a soldering iron guide 326. The soldering iron guide 326 may be connected to the sleeve device 308 via the dowels 318A and 318B, and it may have openings 328A and 328B (generally, opening 328) which function similarly to the openings 322A and 322B. One difference between the soldering iron guide 326 and the lancing guide 320 is that the openings 330A and 330B (generally, opening 330) are sized and shaped to fit an end of a soldering iron, as opposed to the pointed end 334 of the lancet 332. The heated point of a soldering iron may then be inserted into the openings 330A and 330B in order to create openings in the housing of the ingestible device 300. Similar to the lancet 332, the heated point of the soldering iron may be heated to a temperature that is above a melting point of a material used to create the ingestible device 300.

In some embodiments, the openings 324A and 324B may be replaced with any number of openings, including a single opening at the top of the lancet guide. In some embodiments, there may be no aperture 310, no fitting screw 312, no feature or marking 302 on the ingestible device 300, or any suitable combination thereof. For example, if there is only a single sampling chamber within the ingestible device 300, or if the precise positioning of the opening created in the ingestible device 300 is unimportant, there may be no need for the ingestible device 300 to be oriented a particular way relative to the sleeve device 308.

In general, the systems and methods discussed in relation to FIG. 3 may be modified or altered to accommodate the particular ingestible device and the particular application that the opening in the ingestible device is needed for. This may include, for example, incorporating the systems and methods discussed in relation to FIG. 3 into one or more automated systems for creating openings in an ingestible device, which may be operated by an appropriate combination of sensors, actuators, microcontrollers, mechanized systems, robotic systems, and the like. Additionally, the systems and methods discussed in relation to FIG. 3 may be combined with any other systems and methods, including the systems and methods discussed in relation to FIG. 1, FIG. 2, or FIGS. 4-9. For example, after creating openings in the ingestible device 300, the sleeve device 308 may be connected to or more tubes by a base portion, similar to the base portion 112 of the sleeve device 108 depicted in FIG. 1, or the base portion 508 which is discussed in relation to FIG. 5. The ingestible device 300, the sleeve device 308, and the attached base portion connected to tubes may then be inserted into a centrifuge in order to extract a portion of the sample from the ingestible device, similar to the method discussed in relation to FIG. 1. As an alternate example, the lancet 332 may be replaced with a modified version of the lancing mechanism 206 discussed in relation to FIG. 2. The modified version of the lancing mechanism 206 may be designed to be inserted into the openings 324A and 324B simultaneously. As yet another example, a modified version of the lancet 332 may be configured to automatically retract the pointed end 334 of the lancet 332 once it is fully inserted into the opening 324A. This may be done, for example, through a spring-loaded mechanism, which is triggered when the base of the lancet 332 (i.e., the point where the pointed end 334 of the lancet 332 is connected to the remaining portion of the lancet 332) comes into contact with the top of the lancing guide 320.

FIG. 4 shows an illustrative embodiment of a grating device, which may be used to create an opening in an ingestible device, and may be used in conjunction with the method illustrated by FIG. 1, or which may be combined either wholly or in part with any of the systems and methods discussed in relation to FIG. 2, FIG. 3, or FIGS. 5-9. In general, the grating device may remove a portion of a housing of the ingestible device 400, exposing one or more sampling chambers contained within the ingestible device 400. Diagram 450 shows an ingestible device 400 that is loaded into the grating device. Diagram 452 shows a view from one end of the grating device, after the ingestible device 400 has been loaded into the grating device. Diagram 454 shows a profile view of the grating device, and indicates a motion of the slide block 410 used to remove a portion of the housing of the ingestible device 400. Diagram 456 shows the ingestible device 400, before and after the grating device is used to remove a portion of the housing of the ingestible device 400.

As shown in diagram 450, the grating device includes a holder 406 having a built-in platform 402, which includes a recessed area 404 that is sized and shaped to snugly hold a bottom portion of the ingestible device 400. In some embodiments, the platform 402 may be removable from the holder 406, which may allow the ingestible device to be easily inserted and oriented within the recessed area 404 before the platform 402 is connected to the rest of the grating device. The ingestible device 400 is partially inserted into the recessed area 404 of the holder 406, such that the top portion of the ingestible device is exposed.

The side of the holder 406 forms a rail 412A, and the other side of the holder 406 may form a second rail 412B (shown in diagram 452). The grating device includes a slide block 410 which is configured to move along the rails 412A and 412B (generally, rail 412), and pass over the exposed top portion of the ingestible device 400.

Diagram 452 shows a view from one end of the grating device. As shown in diagram 452, two grating surfaces 416A and 416B (generally, grating surface 416) are connected to the inside of the slide block 410. The grating surfaces 416A and 416B may be positioned so that when the slide block 410 passes over the exposed top portion of ingestible device 400, the grating surfaces 416A and 416B scrape against the top portion of the ingestible device 400. This scraping may remove a portion of the housing of the ingestible device 400, potentially exposing one or more sampling chambers within the ingestible device. It will be understood that the slide block 410 may be passed over the exposed top portion of the ingestible device 400 several times before enough of the housing is removed for the sampling chambers within the ingestible device 400 to be exposed.

In general, the grating surfaces 416A and 416B may be made of any type of sufficiently sharp blade, or sufficiently abrasive material. For example, the grating surfaces 416A and 416B may include grated blades formed from metal or ceramic, a roughened or abraded surface similar to a rasp or file, or other types of rough materials, such as sand paper or Emory cloth.

In diagram 452, the grating surfaces 416A and 416B are depicted as oriented substantially perpendicularly to each other. This may help to simplify the geometry of the slide block 410, and may allow the grating device to expose two sampling chambers within the ingestible device simultaneously. In some embodiments, the orientation of the grating surfaces 416A and 416B may have a different orientation relative to one another, in order to accommodate the shape and size of the ingestible device 400, or to alter the location of the portion of the housing of the ingestible device 400 that is removed.

The grating surfaces 416A and 416B may be connected to the slide block 410 any number of ways. For example, the grating surfaces 416A and 416B may be connected by magnetic plates attached to the inside of the slide block 410, or by mechanical fasteners such as bolts, screw, springs, and the like. Certain types of connections, such as the use of magnetic plates, may allow the grating surfaces 416A and 416B to be removed from the slide block 410. In turn, this may allow the grating surfaces 416A and 416B to be periodically replaced with new grating surfaces once old grating surfaces wear down from use. Two spring mechanisms 418A and 418B (generally, spring mechanism 418) are positioned between the grating surfaces 416A and 416B and the interior wall of the slide block 410. The spring mechanisms 418A and 418B may push the grating surfaces 416A and 416B towards the ingestible device 400 when the slide block 410 is passing over the top portion of the ingestible device 400. This may allow the grating device to accommodate different types of ingestible devices, to ensure that a new portion of the housing of the ingestible device 400 is removed each time that the slide block 410 moves over the ingestible device 400, or to compensate for slight variations for how firmly the ingestible device 400 is seated into the recessed area 404 of the holder 406. However, in some embodiments, the spring mechanisms may not be included in the grating device, and the grating surfaces 416A and 416B are simply connected to the slide block 410. In some embodiments, the spring mechanisms 418A and 418B only push the grating surfaces 416A and 416B to a predetermined depth. For example, the grating surfaces 416A and 416B may be connected to a certain type of fastener that only allows the grating surfaces 416A and 416B to move a certain distance away from the inner wall of the slide block 410. This may, in turn, restrict the amount of material that may be remove from the housing of the ingestible device 400, and limit the size of any openings created in the housing of the ingestible device 400.

Diagram 452 shows the holder 406 connected to the base 408 of the grating device. Rails 412A and 412B extending from the sides of the holder 406 are suspended slightly above the base 408, forming small gaps 422A and 422B (generally gap 422). The slide block 410 has two groove-shaped slots 414A and 414B (generally, groove-shaped slot 414) that extend along the length of the slide block 410. The groove-shaped slots 414A and 414B are shaped to fit at least partially around the rails 412A and 412B (e.g., around three surfaces) as the slide block 410 moves along the rails 412A and 412B, and may guide the motion of the slide block 410. Generally, the movement of the slide block 410 is restricted by the two rails 412A and 412B, and the slide block 410 may move along the two rails 412A and 412B simultaneously as the slide block 410 is passed over the exposed top surface of the ingestible device 400.

Diagram 454 shows a profile view of the grating device, and indicates the motion of the slide block 410 used to remove a portion of the housing of the ingestible device 400. The slide block 410 may be positioned at one side of the base 408, and held in place by the rails 412A and 412B on the sides of the holder 406. The slide block 410 may slide along the rails 412A and 412B, moving along the length of the base 408. This may allow the grating surfaces 416A and 416B to scrape against the exposed top portion of the ingestible device 400. The slide block 410 may be moved back and forth along the rails 412A and 412B any number of times, and this may allow additional portions of the housing of the ingestible device 400 to be removed as the grating surfaces 416A and 416B repeatedly scrape along the top portion of the ingestible device 400.

Diagram 456 shows the ingestible device 400, before and after the grating device is used to remove a portion of the housing of the ingestible device 400. When enough of the housing of the ingestible device 400 is removed, two openings 420A and 420B (generally, opening 420) are created in the housing of the ingestible device. Each of these openings 420A and 420B may expose sampling chambers within the ingestible device 400. Once the openings 420A and 420B have been created, a portion of the sample contained in the sampling chambers may be extracted from the ingestible device 400. This may be done using any appropriate system or method, such as the systems and methods discussed in relation to FIG. 1. For example, the ingestible device 400 may be connected to a sleeve device, which connects the openings 420A and 420B to a set of tubes, similar to the sleeve device 108 and tubes 114A and 114B discussed in relation to FIG. 1. At that point, the ingestible device 400 and the sleeve device may be placed into a centrifuge tube, and a portion of the sample may be transferred from the ingestible device 400 to the tubes by centrifuging the ingestible device 400, the sleeve device, and the tubes.

In some embodiments, the holder 406 or the platform 402 may include a set of orientation markings near the recessed area 404. These orientation markings may indicate the portion of the housing of ingestible device 400 that is removed when the slide block 410 passes over the exposed top portion of the ingestible device 400. This may be particularly advantageous if it is known where the sampling chambers within the ingestible device 400 are located, and if there are any noticeable features or markings on the ingestible device 400 which may be aligned with the orientation markings.

For illustrative purposes, FIG. 4 depicts two rails 412A and 412B, and two groove-shaped slots 414A and 414B. However, in some embodiments there may be only one rail on the holder 406, or one groove-shaped slot on the slide block 410. In these embodiments, it may be advantageous to change the shape of the rail and the groove-shaped slot, to ensure that a single rail is sufficient to restrict the motion of the slide block 410. For example, in some embodiments, a “T”-shaped rail may be used to further restrict the motion of the slide block 410.

In some embodiments, the positioning or shape of the rails 412A and 412B may be different, and the shape and location of the groove-shaped slots 414A and 414B may be altered to match. For example, two “T”-shaped rails may come directly up from the base 408, and the groove-shaped slots 414A and 414B slots may be reshaped accordingly and repositioned to the bottom of the slide block 410.

For illustrative purposes, FIG. 4 depicts two grating surfaces 416A and 416B. However, in some embodiments there may be only one grating surface. If multiple sampling chambers within the ingestible device 400 need to be exposed, it may be possible to use a single grating surface to expose a first sampling chamber, reposition the ingestible device 400 within the holder 406, and then use the same grating surface to expose another sampling chamber within the ingestible device 400. This process may be repeated several times over, as necessary, to expose any number of sampling chambers within the ingestible device 400. Alternatively, the grating device may include more than two grating surfaces 416.

In general, the systems and methods discussed in relation to FIG. 4 may be modified or altered to accommodate different types of ingestible device, or accommodate the particular application that necessitate exposing a sampling chamber within the ingestible device. For example, if there is only a single sampling chamber located within the top of the ingestible device, the grating device may be modified to include only a single grating surface, and the position of the grating surface within the slide block 410 may be repositioned accordingly. As an alternate example, in some embodiments the slide block 410 is in a fixed position relative to the base 408, and the holder 406 or the platform 402 is able to be moved in order to bring the ingestible device 400 in contact with a grating surface 416 within the slide block 410. In some embodiments, the holder 406 may be replaced with a conveyer belt, to move the ingestible device 400 into contact with the grating surface 416 within the slide block 410.

Additionally, the systems and methods discussed in relation to FIG. 4 may be combined with any other systems and methods, including automated systems, and the systems and methods discussed in relation to FIGS. 1-3, or FIGS. 5-9. For example, the orientation of the ingestible device 400 within the holder 406 may be improved by first placing the ingestible device 400 into a sleeve device, similar to sleeve device 308 discussed in connection with FIG. 3. The orientation of the ingestible device 400 within the sleeve device 308 may be guaranteed by use of an aperture and fitting screw (e.g., the aperture 310 and fitting screw 312 of FIG. 3), and the recessed area 404 may be sized and shaped so the sleeve device only fits into the recessed area in a particular orientation. As another example, the movement of the slide block 410 may be automated, and an appropriately configured control system may ensure that the slide block 410 continues to move back and forth over the top of the ingestible device 400 until a predetermined portion of the housing of the ingestible device 400 is removed.

FIG. 5 shows an illustrative embodiment of a sleeve device, which may connect an ingestible device to one or more tubes, and may be used in conjunction with the method illustrated by FIG. 1, or which may be combined either wholly or in part with any of the systems and methods discussed in relation to FIGS. 2-4 or FIGS. 6-9. Diagram 550 shows an ingestible device 500 with two sampling chambers 502A and 502B (generally, sampling chamber 502) inserted into a sleeve device 504, which is used to connect openings in the housing of the ingestible device (not shown) to the tubes 510A and 510B (generally, tube 510). Diagram 552 shows a detailed view of an example base portion 508, which may be included in the sleeve device 504.

Sleeve device 504 connects to the end of the ingestible device 500, and may generally be used in extracting samples from the ingestible device 500. Ingestible device 500 is shown with sampling chambers 502A and 502B, and openings (not shown) in the portion of the housing of the ingestible device inserted into the sleeve device 504 lead to the sampling chambers 502A and 502B. The sleeve device 504 has a sleeve portion 506 and a base portion 508. The sleeve portion 506 is shaped to fit snugly around the circumference of the ingestible device, and the base portion 508 connects the openings in the bottom of the ingestible device 500 to the tubes 510A and 510B.

The top surface of the base portion 508 has openings 516A and 516B (shown in diagram 552), each of which may connect to an opening in the housing of the ingestible device 500. Tunnels run through the base portion 508, and connect the openings 516A and 516B on the top surface of the base portion 508 to similar openings (not shown) in the bottom surface of the base portion 508.

In general, the tubes 510A and 510B are removable tubes, which may be attached or detached from the bottom of the base portion 508. Each of the tubes 510A and 510B have a tube opening, which may face the openings on the bottom of the base portion 508 when the tubes 510A and 510B are attached to the bottom of the base portion 508.

The top surface of the base portion 508 may be connected to the end of the sleeve portion 506 in order to form the sleeve device 504. This connection may be made any number of ways. For example, the openings 520A and 520B (generally, opening 520) may be sized and shaped to accommodate a dowel or a bolt, and the sleeve portion 506 may be connected to the base portion 508 through the use of an appropriately sized dowel or bolt. In this example, the sleeve portion 506 may also have dowel-shaped openings, similar to the sleeve device 308 discussed in relation to FIG. 3, and the sleeve portion 506 and the base portion 508 may be connected by a dowel joint. In some embodiments, another type of mechanism may be used to connect the sleeve portion 506 and the base portion 508. For example, part of the base portion 508 may form a pair of pegs, which fit into the openings 520A and 520B, which allow the sleeve portion 506 and the base portion 508 to be connected together, similar to a mortise and tenon joint. In general, it may be possible to use any type of convenient mechanism, such as magnetic or mechanical fasteners, to connect the sleeve portion 506 and the base portion 508. In some embodiments, depending on the mechanism being used to connect the sleeve portion 506 and the base portion 508, the openings 520A and 520B may not be included on the base portion 508.

In some embodiments, the openings 520A and 520B on the base portion 508 may be sized and shaped to accommodate a dowel, and dowel-shaped tunnels connect the openings 520A and 520B to similar dowel-shaped openings on the bottom of the base portion 508. The sleeve portion 506 may have similar dowel-shaped openings, which connect to another set of dowel-shaped tunnels that extend into the sleeve portion 506 (e.g., similar to the sleeve device 308 of FIG. 3). This configuration may allow a dowel to be inserted all the way through the base portion 508, through the openings on the bottom of the base portion 508, and connect the base portion 508 to the sleeve portion 506.

The top surface of the base portion 508 includes a sealing portion 512, that is sized and shaped to fit the end of the ingestible device 500. The sealing portion 512 forms watertight seals between openings in the housing of the ingestible device and the openings 516A and 516B. These watertight seals may be created by the protruding portions 514A and 514B (generally, protruding portion 514). These protruding portions 514A and 514B may be sized and shaped to fit the curvature of the ingestible device. When the end of the ingestible device 500 is inserted into the base portion 508, each of the protruding portions 514A and 514B may contact a portion of the housing of the ingestible device 500 that surrounds one of the openings on the end of the housing of the ingestible device 500. This may cause each of the protruding portions 514A and 514B to form a watertight seal that encircles the openings in the housing in the ingestible device 500.

In some embodiments, the protruding portions 514A and 514B are able to compress and conform to the shape of the ingestible device 500. For example, the protruding portions 514A and 514B, or the entire base portion 508, may be made from silicone. The shape of the silicone protruding portions 514A and 514B may deform slightly to accommodate the portion of the ingestible device 500 that contacts the protruding portions 514A and 514B. This may help the protruding portions 514A and 514B form watertight seals around the openings in the housing of the ingestible device 500.

Diagram 552 shows two magnets 518A and 518B (generally, magnet 518) included as part of the base portion 508. These magnets may be used to connect the tubes 510A and 510B to the bottom of the base portion 508 of the sleeve device 504. This, in turn, may allow the tubes 510A and 510B to be easily connected or disconnected from the sleeve device. In some embodiments, base portion 508 may not include magnets 518A and 518B, and some other suitable type of mechanism may be used to connect the tubes 510A and 510B to the sleeve device 504. For example, this may be accomplished by mechanically fastening the tubes 510A and 510B to the base portion 508 of the sleeve device 504. In some embodiments, the bottom (not shown) of the base portion 508 is shaped to conform to the opening of the tubes 510A or 510B. For example, there may be a slight protrusion on the bottom of the base portion 508, which is sized and shaped to fit snugly around the outer circumference of the tubes 510A and 510B, or to fill the inner circumference of the tubes 510A and 510B. This may help to ensure that the tubes 510A and 510B are properly oriented relative to the base portion 508, and reduce the chance of any of the sample from within the ingestible device 500 spilling out from the tubes 510A and 510B.

In some embodiments, a window or aperture on the side of the sleeve portion 506 of the sleeve device 504 may be used to view the ingestible device 500 and orient the ingestible device 500 within the sleeve device 504. This may be done in order to ensure that any openings within the housing of the ingestible device 500 are aligned with the openings 516A and 516B in the base portion 508. In some embodiments, the orientation of the ingestible device 500 relative to the sleeve device 504 may be fixed. For example, a peg or fitting screw may be inserted into an aperture on the side of the sleeve portion 506 in order to prevent the orientation of the ingestible device 500 from changing. As another example, the ingestible device 500 and the sleeve device 504 may be shaped such that the ingestible device 500 can only be inserted into the sleeve device 504 in a predetermined orientation. This may help to ensure that openings in the housing of the ingestible device 500 are properly aligned with the protruding portions 514A and 514B of the base portion 508.

For illustrative purposes, FIG. 5 depicts two sampling chambers within the ingestible device 500, two openings 516A and 516B within the base portion 508 of the sleeve device 504, and two tubes 510A and 510B. However, the general size and shape of the sealing portion 512 and the base portion 508 may be modified to connect any number of sampling chambers to any number of tubes.

In general, the systems and methods discussed in relation to FIG. 5 may be modified or altered to accommodate different types of ingestible devices. For example, it is understood that the general design and shape of the sleeve device 504 may be easily modified to accommodate different types of ingestible devices, openings in the housing of an ingestible device which are located in different positions around the ingestible device. For example, if there is only a single sampling chamber located within the top of an ingestible device, the openings 516A and 516B may be replaced with a single opening in the center of the sealing portion 512, and this opening may connect the ingestible device to a single tube.

Additionally, the systems and methods discussed in relation to FIG. 5 may be combined with any other systems and methods, including automated systems, and the systems and methods discussed in relation to FIGS. 1-4, or FIGS. 6-9. For example, the orientation of the ingestible device 500 may be fixed within the sleeve device 504 by inserting a positioning pin or a fitting screw into an aperture on the side of the sleeve portion 506 of the sleeve device 504 (e.g., similar to the aperture 310 and fitting screw 312 of FIG. 3). As an alternate example, the sleeve device 504 may be modified to accommodate any ingestible device with an opening or exposed sampling chamber, such as the ingestible devices 100, 200, 300, and 400. Additionally, any ingestible device connected to sleeve device 504 may be inserted into a centrifuge in order to extract a portion of the sample from the ingestible device, similar to the methods discussed in relation to FIG. 1.

FIG. 6 shows illustrative diagrams of a centrifuge mechanism, which may allow a sleeve device and an ingestible device to be inserted into a centrifuge, and may be used in conjunction with the method illustrated by FIG. 1, or which may be combined, either wholly or in part, with any of the systems and methods discussed in relation to FIGS. 2-5 or FIGS. 7-9. Diagram 650 shows an exploded view of the centrifuge mechanism, and shows details of the different components which may be included in the centrifuge mechanism. Diagram 652 shows a cross-sectional view of the assembled centrifuge mechanism. Diagram 654 shows a detailed view of the interface between an ingestible device and a sleeve device when placed together within the centrifuge mechanism.

The ingestible device 600 is placed within a sleeve portion 602, and the end of the ingestible device 600 rests at the top of a base portion 604. Together, the sleeve portion 602 and the base portion 604 form a sleeve device 628, and are able to connect the ingestible device 600 to two tubes 606A and 606B (generally, tube 606). In some embodiments, these elements (e.g., the ingestible device 600, the sleeve portion 602, the base portion 604, and the tubes 606) may be the same as the ingestible device 500, the sleeve portion 506 and base portion 508 that form the sleeve device 504, and tubes 510A and 510B discussed in relation to FIG. 5. In general, the sleeve portion 602 and the base portion 604 may be made of any suitable material, such as machined polycarbonate or molded silicone.

The ends of the tubes 606A and 606B are placed into a tube holder 608, and the tip of the tube holder 608 may be sized and shaped to fit snugly within the end of the centrifuge tube 610. The tube holder 608 may have an extended portion 626, which comes up from the tube holder 608 and may be positioned near the top of the centrifuge tube 610 when the centrifuge mechanism is fully assembled. Pulling on the extended portion 626 may be used to remove the contents of the centrifuge tube 610. This may be particularly useful if the centrifuging mechanism needs to be disassembled once the centrifuging has been completed.

Once the ingestible device 600, the sleeve device 628, the tubes 606A and 606B, and the tube holder 608 are assembled together within the centrifuge tube 610, a cap 612 may be used to cover the centrifuge tube. The bottom surface of the cap 612 is connected to a spring 614, and a brace 616 extends from the bottom of the cap 612. The spring 614 may be used to help maintain the position of the ingestible device 600 relative to the sleeve device 628 while the ingestible device 600 is inserted into the sleeve device 628. This may be done by using a spring 614 that is sized and shaped to fill the space between the cap 612 and ingestible device 600, and apply a downward pressure to the ingestible device 600. The brace 616 is sized and shaped to fill the space around the sides of the ingestible device 600, between the cap 612 and the sleeve device 628. This may help hold the ingestible device 600 within position in the center of the centrifuge tube 610, and also help to maintain the position of the sleeve device 628 and the tube holder 608 at the bottom of the centrifuge tube 610.

In some embodiments, the spring 614 is not attached to the cap 612, and is a separate component. For example, the spring 614 may be placed onto the ingestible device 600 after the ingestible device 600 and the sleeve device 628 have been inserted into the centrifuge tube 610. The cap 612 may then be screwed or fitted onto the top of the centrifuge tube 610. This may compress the spring 614, and cause the spring 614 to push the ingestible device 600 towards the bottom of the centrifuge tube 610.

As shown in diagram 654, an opening 624 in the housing of the ingestible device 600 is pressed against an opening 622 in the base portion 604 of the sleeve device 628 when the ingestible device 600 and the sleeve device 628 are connected together. A protruding portion 630 of the base portion 604 contacts the housing of the ingestible device 600 around the opening 624, and forms a seal between the opening 624 in the housing of the ingestible device 600, and the opening 622 in the base portion 604. This may be similar to the system discussed in relation to FIG. 5, where the protruding portions 514A and 514B of the sealing portion 512 of the base portion 508 contact the ingestible device 500 to form a watertight seal.

A tunnel 620A runs through the base portion 604, and connects the opening 622 on the top surface of the base portion 604 to a similar opening on the bottom surface of the base portion 604. This may form path for a portion of the sample to flow through when it is transferred from the sampling chamber 618A into the tube 606A. Generally, the tunnels 620A and 620B (generally, tunnel 620) are able to connect the sampling chambers 618A and 618B (generally, sampling chamber 618) to one or more tubes.

After the centrifuge mechanism is assembled, the centrifuge tube 610 containing the ingestible device 600, the sleeve device 628, and the tubes 606A and 606B may be placed into a centrifuge. When the centrifuge is turned on, the centrifugal forces may force any sample contained within the sampling chambers 618A and 618B in a downward direction, towards the bottom of the centrifuge tube 610. This may force the samples out of the sampling chambers 618A and 618B, through the tunnels 620A and 620B in the base portion 604 of the sleeve device 628, and into the tubes 606A and 606B.

After a sufficient amount of the sample has been transferred into the tubes 606A and 606B, the centrifuge may be turned off, the centrifuge mechanism disassembled, and the tubes 606A and 606B may be recovered by detaching them from the base portion 604.

For illustrative purposes, FIG. 6 depicts two sampling chambers 618A and 618B within the ingestible device 600, two tunnels 620A and 620B running through the base portion 604, and two tubes 606A and 606B being used to collect the sample. However, the centrifuge mechanism may be modified to connect any number of sampling chambers to any number of tubes through any number of tunnels.

For illustrative purposes, FIG. 6 depicts a centrifuge mechanism that includes a spring 614 and a brace 616. However, in some embodiments, the spring 614 and/or the brace 616 may not be included. In these embodiments, centrifugal forces alone may be enough to hold the ingestible device 600 within the sleeve device 628. Similarly, the general size and shape of the sleeve device 628 may be altered to fit snugly with the centrifuge tube 610, or to better secure the ingestible device within the centrifuge tube 610.

In some embodiments, when the sample is to be collected in the bottom of the centrifuge tube 610 instead of the tubes 606A and 606B, the tubes 606A and 606B and the tube holder 608 may not be included in the centrifuge mechanism. It is also possible that the design of the sleeve device 628 may be altered accordingly. For example, the sleeve device 628 may be redesigned to simply suspend the ingestible device 600 above the bottom of the centrifuge tube 610, with an opening in the housing of the ingestible device 600 facing towards the bottom of the centrifuge tube 610.

In general, the systems and methods discussed in relation to FIG. 6 may be modified or altered to accommodate different types of ingestible devices. For example, it is understood that the general design and shape of the sleeve device 628 may be easily modified to accommodate different types of ingestible devices, or openings in the housing of an ingestible device which are located in different positions around the ingestible device. For example, if there is only a single sampling chamber located within the bottom of an ingestible device, a single tunnel running through the base portion 604 may connect an opening in the bottom of the ingestible device to a single tube.

Additionally, the systems and methods discussed in relation to FIG. 6 may be combined with any other systems and methods, including automated systems, and the systems and methods discussed in relation to FIGS. 1-5, or FIGS. 7-9. For example, the sleeve device 504 discussed in relation to FIG. 5 may be incorporated into the centrifuge mechanism as the sleeve device 628. As an alternate example, the centrifuge mechanism may, as a whole, be used to replace the centrifuge tube 116 discussed in relation to FIG. 1. It will also be understood that any of the systems or methods discussed in relation to FIG. 6 may be combined, either wholly or in part, with the centrifuge mechanism discussed in relation to FIG. 7.

FIG. 7 shows an illustrative diagram of a centrifuge mechanism, which may allow a sleeve device and an ingestible device to be inserted into a centrifuge, and may be used in conjunction with the methods described in relation to FIG. 1. The centrifuge mechanism of FIG. 7 includes several modifications which may be made to the centrifuge mechanism discussed in relation to FIG. 6, and may be combined, either wholly or in part, with any of the systems and methods discussed in relation to FIGS. 2-6, FIG. 8, or FIG. 9. Diagram 750 shows an internal view of an assembled centrifuge mechanism, which includes a modified sleeve device. Diagram 752 shows an external view of the same centrifuge mechanism shown by diagram 750.

The ingestible device 700 is positioned within a sleeve portion 702, and the end of the ingestible device 700 rests at the top of a base portion 704. Together, the sleeve portion 702 and the base portion 704 form a sleeve device 718, and connect the ingestible device 700 to a tube 706. The ingestible device 700, the sleeve device 718, and the tube 706 may be similar to the arrangement of the ingestible device 600, the sleeve device 628, and the tubes 606A and 606B discussed in relation to FIG. 6.

The end of the tube 706 is placed in a tube holder 720, and the tip of the tube holder 720 may be sized and shaped to fit snugly within the end of the centrifuge tube 712. The centrifuge tube 712 is covered by a cap 714. Similar to cap 612 discussed in relation to FIG. 6, cap 714 is connected to a spring 716 and a brace 722. The spring 716 may be used to help maintain the position of the ingestible device 700 relative to the sleeve device 718, and the brace 722 is sized and shaped to fill the space around the sides of the ingestible device 700, between the cap 714 and the sleeve device 718.

On the side of the sleeve portion 702 is an aperture 708, which may be used to view the orientation of the ingestible device 700 within the sleeve device 718. A moveable fitting screw 710 is inserted into aperture 708, and may be used to restrict the motion of the ingestible device 700 relative to the sleeve device 718. In some embodiments, the moveable fitting screw 710 is inserted through the side of the centrifuge tube 712, and the moveable fitting screw 710 may be used to restrict the motion of the ingestible device 700 relative to the centrifuge tube 712 as well. In some embodiments, the moveable fitting screw 710 is made of a transparent material. This may allow the orientation of the ingestible device 700 within the sleeve device 718 to be viewed through the aperture 708 when the moveable fitting screw 710 is inserted into the aperture 708.

For illustrative purposes, FIG. 7 shows a single tube 706 being used to collect samples from the ingestible device 700. If the ingestible device 700 has multiple sampling chambers, multiple tunnels may run through the base portion 704, each tunnel connecting a different sampling chamber to the same tube 706 through openings in the housing of the ingestible device 700. Furthermore, in some embodiments, the base portion 704 may be modified to connect any number of sampling chambers within the ingestible device 700 to any number of tubes.

In general, the systems and methods discussed in relation to FIG. 7 may be modified or altered to accommodate different types of ingestible devices. For example, it is understood that the general design and shape of the sleeve device 718 may be easily modified to accommodate different types of ingestible devices, or accommodate openings in the housing of an ingestible device which are located in other positions.

Additionally, the systems and methods discussed in relation to FIG. 7 may be combined with any other systems and methods, including the systems and methods discussed in relation to FIGS. 1-6, FIG. 8, or FIG. 9. For example, any of systems and methods of the centrifuge mechanism discussed in relation to FIG. 6 may be applied to the centrifuge mechanism of FIG. 7, and the individual components used in different centrifuge mechanism embodiments may be interchangeable with one another. As an alternate example, the sleeve device 718 may be replaced with any other suitable type of device connecting sampling chambers of an ingestible device to an appropriately sized tube, and the size and shape of the other components of the centrifuge mechanism may be adapted accordingly. Examples of other systems and methods for accessing sampling chambers of an ingestible device and connecting the sampling chambers to tubes, which may be incorporated into a centrifuge mechanism similar to the mechanisms discussed in relation to FIG. 6 and FIG. 7, are discussed in relation to FIG. 8 and FIG. 9.

FIG. 8 shows an illustrative embodiment of a method for separating the ingestible device into multiple portions, and extracting a sample from a portion of the ingestible device. The method includes separating the ingestible device into a first portion and a second portion, the first portion comprising a sampling chamber that contains at least a portion of the sample. An example system and method for separating an ingestible device into portions is shown in diagrams 850, 852, and 854. The method also includes connecting the sampling chamber to an adapter. Connecting the sampling chamber to the adapter is shown in diagram 856, and an example adapter is discussed in relation to FIG. 9. The adapter, in turn, may be connected to a tube. The method includes centrifuging the first portion of the ingestible device, while the sampling chamber is connected to the adapter, to transfer at least the portion of the sample from the sampling chamber into the tube. This may be done by inserting the adapter, the ingestible device, and the tube into a centrifuge mechanism (e.g., variations of the centrifuge mechanisms discussed in relation to FIG. 6 and FIG. 7) and making use of a centrifuge similar to the centrifuge 118 discussed in relation to FIG. 1.

As shown in diagram 850, an ingestible device 800 may have a first portion 802A that houses the sampling chambers within the ingestible device, and a second portion 802B. The ingestible device 800 is inserted into a cutting mechanism 806 by positioning the ingestible device 800 within a recessed area 804 of the cutting mechanism 806. The cutting mechanism 806 has a moveable blade 808 which may be operated by means of a handle 810. In general, the first portion 802A of the ingestible device 800 is positioned within the recessed area 804, and the second portion 802B is exposed. The moveable blade 808 may be used to cut the ingestible device 800 and form two separate portions 802A and 802B, while the recessed area 804 holds the ingestible device 800.

As shown in diagram 852, after the ingestible device 800 is inserted into the cutting mechanism 806, a back-piece 812 may be connected to the cutting mechanism 806. The back-piece 812 may further restrain the ingestible device 800, and provide additional resistance when the end of the moveable blade 808 is inserted into the ingestible device 800.

Diagram 854 shows the back-piece 812 and the cutting mechanism 806 connected together. When the back-piece 812 and the cutting mechanism 806 are connected together, the walls of the back-piece 812 and the recessed area 804 form a chamber that holds the ingestible device 800. As noted above, this may effectively restrain the ingestible device 800 while it is being cut by the moveable blade 808, and provide additional resistance when the end of the moveable blade 808 is inserted into the ingestible device 800.

By pushing the handle 810 towards the body of the cutting mechanism 806, the moveable blade 808 is pressed into the ingestible device 800. If sufficient pressure is placed on the handle 810, the moveable blade 808 may cut through the ingestible device 800, separating the ingestible device 800 into a first portion 802A and a second portion 802B. Once the first portion 802A and the second portion 802B have been formed, the back-piece 812 may be disconnected and removed from the cutting mechanism 806. At this point, the second portion 802B may be removed, and the handle 810 may be used to retract the moveable blade 808.

Diagram 856 shows the cutting mechanism 806 after ingestible device 800 has been separated into two portions. Removing the second portion 802B exposes a barrier 814 within the first portion 802A of the ingestible device 800. The barrier 814 may cover the sampling chambers contained within the first portion 802A of the ingestible device 800. In some embodiments, the barrier 814 may form a portion of the outer surface of the sampling chamber, or it may be part of a structure within the first portion 802A that houses the sampling chambers.

After the barrier 814 is exposed, an adapter 816 is inserted into the first portion 802A of the ingestible device 800. The bottom of the adapter 816 may include hollow needles, and inserting the adapter 816 into the first portion 802A may cause the hollow needles to penetrate the barrier 814, and enter one or more sampling chambers within the ingestible device 800. An example of an adapter 816 is discussed in detail in relation to FIG. 9.

In some embodiments, there may visible features or markings on the barrier 814, which indicate the position of the sampling chambers within the first portion 802A. This may ensure that the hollow needles of the adapter 816 are inserted into the correct locations of the first portion 802A. In some embodiments, there may be small indentations on the barrier 814. These indentations may be used to guide the hollow needles of the adapter 816, and the hollow needles of the adapter 816 may penetrate the barrier 814 at the location of the indentations.

After the adapter 816 is inserted into the first portion 802A, one or more tubes (not shown) may be connected to the top of the adapter 816. These tubes may be sized and shaped similar to the tubes 510A and 510B (FIG. 5), and may be connected to the adapter 816 similar to how the tubes 510A and 510B connect to base portion 508, as discussed in relation to FIG. 5. The first portion 802A may then be loaded into a centrifuge tube while it is connected to the adapter 816, and run through a centrifuge (e.g., the centrifuge 118 of FIG. 1). This may cause at least a portion of the sample to be transferred from the sampling chambers within the first portion 802A into the tubes. After the centrifuging is completed, the tubes containing portions of the sample may be disconnected from the adapter 816, and may be used in laboratory testing.

As an alternate example, instead of tubes, the adapter 816 may connect the sampling chambers within the first portion 802A to a vacuum mechanism. This may allow the sample to be directly sucked from the first portion 802A of the ingestible device 800. The sample may then be deposited into a tube or other suitable receptacle attached to the vacuum mechanism. This may be done instead of centrifuging the first portion 802A and the adapter 816, or it may be done either before or after a portion of the sample has been extracted by centrifuging. To assist in this process, the adapter 816 may simultaneously connect a vacuum mechanism and a source of air or flushing fluid to a single sampling chamber within the first portion 802A of the ingestible device 800. This may allow air or flushing fluid to enter the sampling chamber as the sample is being sucked out of the sampling chamber, allowing the sample to be easily sucked out.

In some embodiments, the adapter 816, the first portion 802A, and the tubes are inserted into a centrifuge mechanism prior to being centrifuged. For example, the centrifuge mechanisms discussed in relation to FIG. 6 and FIG. 7 may be modified to hold the adapter 816 while it is connected to an ingestible device, instead of the sleeve devices 628 and 718. More generally, any of the various systems or methods discussed in relation to FIG. 6 and FIG. 7 may be modified to accommodate the first portion 802A and adapter 816. For example, this may include inserting the adapter 816 and the first portion 802A into a centrifuge tube (e.g., the centrifuge tube 610 or 712), and attaching the centrifuge tube to a cap having a spring at the bottom of the cap (e.g., the cap 612 or 714 attached to spring 614 or 716). This may cause the spring to maintain a position of the first portion 802A relative to the adapter 816 while they are centrifuged. As an alternate example, a moveable fitting screw or positioning pin (e.g., the moveable fitting screw 710) may be used to restrict the movement of the first portion 802A relative to the centrifuge tube. This may be done by inserting the moveable fitting screw into an aperture on the side of the adapter 816.

In general, the systems and methods discussed in relation to FIG. 8 may be modified or altered to accommodate different types of ingestible device. For example, the size and shape of the recessed area 804, and the positioning of the ingestible device within the recessed area 804, may be altered. This may change where the moveable blade 808 cuts the ingestible device, and determine where the separation between the first portion and second portion is made. Similarly, the general design of the adapter 816 may be altered to accommodate the size and shape of the ingestible device, and may connect any number of sampling chambers within the ingestible device to any number of tubes. For example, the adapter 816 may have a single hollow needle that connects a single sampling chamber to a single tube, or the adapter 816 may have two hollow needles that connect two sampling chambers to two separate tubes.

Additionally, the systems and methods discussed in relation to FIG. 8 may be combined with any other systems and methods, including automated systems, and the systems and methods discussed in relation to FIGS. 1-7, or FIG. 9. For example, moveable blade 808 may automatically retract into the cutting mechanism 806 when the handle 810 is fully inserted into the cutting mechanism 806, similar to the lancing mechanism 206 and the plunger 208 discussed in relation to FIG. 2. As another example, similar to the heated lancet discussed in relation to FIG. 2, the moveable blade 808 may be heated to a temperature that is above the melting temperature of the housing of the ingestible device 800. This may allow the moveable blade 808 to easily cut into the ingestible device 800, thereby forming the first portion 802A and the second portion 802B. This may be done by incorporating a heating element, such as an electric heater, into the cutting mechanism 806. This may also be done by removing the moveable blade 808 from the cutting mechanism 806, and heating it using an external heat source. As one example, the motion of the moveable blade 808 may be fully automated, and the cutting mechanism 806 automatically cuts the ingestible device 800 into two portions 802A and 802B upon detecting that the back-piece 812 is connected to the cutting mechanism 806. Similarly, an automated system may connect the back-piece 812 to the cutting mechanism 806, remove and dispose of the second portion 802B, and connect the adapter 816 to the first portion 802A. This may be done using an appropriate set of sensors, actuators, microcontrollers, computerized systems, robotic systems, or the like.

FIG. 9 shows an illustrative adapter device, which may connect a portion of an ingestible device to a tube, and may be used in conjunction with the method illustrated by FIG. 8. Furthermore, the adapter device discussed in relation to FIG. 9 may be combined either wholly or in part with any of the systems and methods discussed in relation to FIGS. 2-8.

Diagram 950 shows a detailed external view of the adapter device, and diagram 952 shows a detailed internal cross-sectional view of the adapter device when it is connected to an ingestible device portion 908. As shown in diagram 950, the top surface of the adapter device 900 includes four openings 904A, 904B, 904C, and 904D (generally, opening 904). Each of these openings 904A, 904B, 904C, and 904D are connected to similar openings (e.g., the openings 918A and 918B shown in diagram 952) on the bottom surface of the adapter device 900 by tunnels (e.g., the tunnels 906A and 906B, shown in diagram 952) which run through the body of the adapter device 900. Hollow needles 902A, 902B, 902C, and 902D (generally, hollow needle 902) protrude from the bottom surface of the adapter device 900, and each hollow needle 902A, 902B, 902C, and 902D has a lumen that connects to one of the openings on the bottom surface of the adapter device 900.

For simplicity, diagram 952 only shows two openings 904A and 904B, that connect via two tunnels 906A and 906B (generally, tunnels 906) to two openings 918A and 918B (generally, openings 918). These openings 918A and 918B are connected to two hollow needles 902A and 902B, which connect with sampling chambers 910A and 910B (generally, sampling chamber 910) within the ingestible device portion 908. However, it will be understood that the openings 904C and 904D and the hollow needles 902C and 902B may function in a similar manner, and any of the systems and methods discussed in relation to one of the openings 904 or hollow needles 902 may apply to all of the others.

As shown in diagram 952, tunnel 906A connects the opening 904A on the top surface of the adapter device 900 to the opening 918A on the bottom surface of the adapter device 900. The hollow needle 902A protrudes from the bottom surface of the adapter device 900, and has a lumen connected to the opening 918A. The hollow needle 902A may penetrate the barrier 914A, and connect to the sampling chamber 910A. It is understood that the barriers 914A and 914B (generally, barrier 914) may be penetrated by any hollow needle 902.

The lumen of hollow needle 902A, the opening 918A, the tunnel 906A, and the opening 904A form a fluid channel, and the hollow needle 902A is sized and shaped such that a portion of the sample contained in the sampling chamber 910A is able to flow through this fluid channel. A similar fluid channel is formed by the combination of the lumen of hollow needle 902B, the opening 918B, the tunnel 906B, and the opening 904B, which allows a portion of the sample to flow out from the sampling chamber 910B.

In general, the ingestible device portion 908 may include a wiper 912, which contacts the internal walls of the housing of the ingestible device portion 908, and forms part of the boundaries of the sampling chambers 910A and 910B. The wiper 912 may act as a watertight barrier, isolating samples in the sampling chambers 910A and 910B until the top portion of the wiper 912 that forms the barriers 914A and 914B is penetrated by the hollow needles 902A and 902B. The ingestible device portion 908 is also depicted with a secondary barrier 916. This secondary barrier 916 may act as a fail-safe to ensure that the sample remains with ingestible device portion 908 in case the barriers 914A and 914B formed by the wiper 912 are damaged. The secondary barrier 916 may be constructed from a similar material as the wiper 912, and may be penetrated by the hollow needles 902A and 902B in a similar fashion.

The openings 904A, 904B, 904C, and 904D may be connected with removable tubes, similar to the tubes 510A and 510B connected to the sleeve device 504 in FIG. 5. These tubes may have tube openings which face the openings 904A, 904B, 904C, and 904D when the tubes are connected to the top surface of the adapter device 900. These tubes may be connected to the adapter device by any convenient means, such as through the use of mechanical fasteners or magnets. In some embodiments, the adapter device 900 includes magnets, which may be used to connect tubes to the appropriate locations of the adapter device 900 near the openings 904A, 904B, 904C, and 904D.

In some embodiments, the adapter device 900 may be configured to have two hollow needles simultaneously connected to a single sampling chamber within the ingestible device portion 908. This may allow a sample to be removed from the sampling chamber without the need for centrifuging the ingestible device portion 908 and the adapter device 900. For example, both of the hollow needles 902B and 902C may be connected simultaneously to the sampling chamber 910B. In these embodiments, one hollow needle (e.g., hollow needle 902B) may be used to remove a sample from the sampling chamber 910B, and the other hollow needle (e.g., the hollow needle 902C) may allow air or fluid to enter sampling chamber 910B. For example, the hollow needle 902B may be connected to a vacuum mechanism (e.g., by connecting the vacuum mechanism to the opening 904B), which applies suction to the sampling chamber 910B. At the same time, the hollow needle 902C may allow air to flow into the sampling chamber 910B, preventing negative pressure from building up within the sampling chamber 910B and resisting the force of the suction. As an alternate embodiment, the hollow needle 902C may pump fluid into the sampling chamber 910B, such as water or saline. This may be done by attaching the water source to the opening 904C. This may flush out the sampling chamber 910B, causing any of the sample contained in the sampling chamber 910B to be forced out of the other hollow needle 902B.

In some embodiments, the adapter device 900 may be used to load substances into the ingestible device portion 908. For example, after a hollow needle 902 has been inserted into the ingestible device portion 908, the substance may be inserted into the ingestible device portion 908 through the opening 904. The opening 904 may be connected to a funnel, tube, needle, syringe, or any other mechanism which allows substances to be placed into the opening 904. Afterwards, the ingestible device portion 908 may be connected to another ingestible device portion, in order to form a single ingestible device that may be administered to a patient. In general, this method may allow any type of substance to be loaded into an ingestible device through the use of the adapter device 900.

In some embodiments, one or more of the hollow needles 902A, 902B, 902C, and 902D may include a retractable sleeve around the needle. After a hollow needle (e.g., hollow needle 902A) is inserted into a sampling chamber (e.g., the sampling chamber 910A), the sleeve around the hollow needle 902A is retracted. This may result in the hollow needle 902A penetrating the barrier 914A through an opening which is slightly wider than the hollow needle 902A. This may provide a path for air to enter the sampling chamber 910A as the hollow needle 902A is used to remove the sample from the sampling chamber 910A. For example, this may be particularly advantageous if the opening 904A connected to the hollow needle 902A is connected to a vacuum mechanism which sucks a portion of the sample out of the sampling chamber 910A.

In some embodiments, one or more secondary needles may be inserted through the hollow needles 902A, 902B, 902C, and 902D. After a hollow needle (e.g., hollow needle 902A) is inserted into a sampling chamber (e.g., the sampling chamber 910A), a thinner secondary needle is inserted through the hollow needle and into the sampling chamber. This secondary needle may be inserted, for example, through one of the openings 904 of the top surface of the adapter device 900. The secondary needle may then be used to extract the sample from the sampling chamber 910A, and the area between the outer circumference of the secondary needle and the inner circumference of the hollow needle 902A may provide a path for air to enter the sampling chamber 910A.

For illustrative purposes, the adapter device 900 of FIG. 9 is depicted with the top surface of the adapter device 900 being divided into an upper level and a lower level. The openings 904A and 904B are located on the upper level, and the openings 904C and 904D are located on the lower level. This may visually distinguish the different openings 904, or allow only certain types of devices or mechanisms to be connected to each of the openings 904. For example, a vacuum mechanism may be configured to only connect to openings on the upper level, and a source of flushing fluid may by configured to only connect to openings on the lower level. However, in some embodiments, the design and shape of the adapter device 900 may be altered, and all of the openings 904 may be placed on a single level.

In general, the systems and methods discussed in relation to FIG. 9 may be modified or altered to accommodate different types of ingestible devices. For example, it is understood that the general design and shape of the adapter device 900 may be modified to accommodate different types of ingestible devices, and the number and positioning of the various openings and hollow needles may be altered to accommodate the number and position of the sampling chambers within the ingestible device portion. For example, if there is only a single chamber located in the center of the ingestible device, there may be only a single hollow needle extending from center of the bottom of the adapter device 900. As an alternate example, in some embodiments the hollow needles can penetrate the housing of an ingestible device. This may allow the adapter device 900 to connect to an ingestible device without first separating the ingestible device into a first and second portion.

Additionally, the systems and methods discussed in relation to FIG. 9 may be combined with any other systems and methods, including automated systems, and the systems and methods discussed in relation to FIGS. 1-8. For example, the adapter device 900 may be directly incorporated into the systems and methods discussed in connection with FIG. 8 as the adapter 816. As an alternate example, the adapter device 900 may be used in a similar fashion as the sleeve device 504 discussed in relation to FIG. 5, in order to connect an ingestible device to one or more tubes. The adapter device 900, may be included as part of the centrifuging mechanisms discussed in relation to FIG. 6 and FIG. 7, and the systems and methods discussed in relation to FIG. 1 may be adapted to centrifuge the adapter device 900, a portion of an ingestible device, and a tube, in order to transfer a portion of the sample within the portion of the ingestible device into the tube.

Moreover, while examples have been provided in which holes are generated at the top of an ingestible device, other approaches are also within the disclosure.

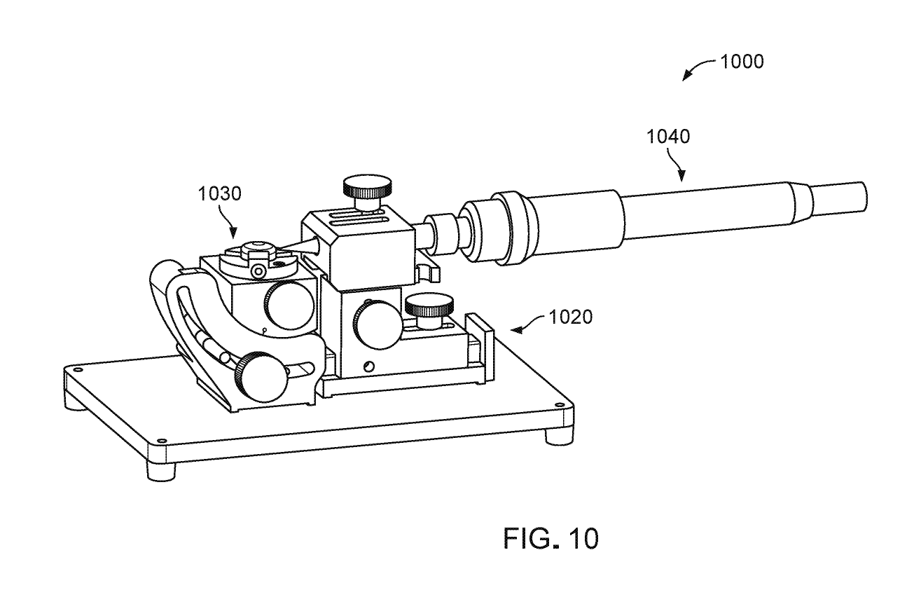

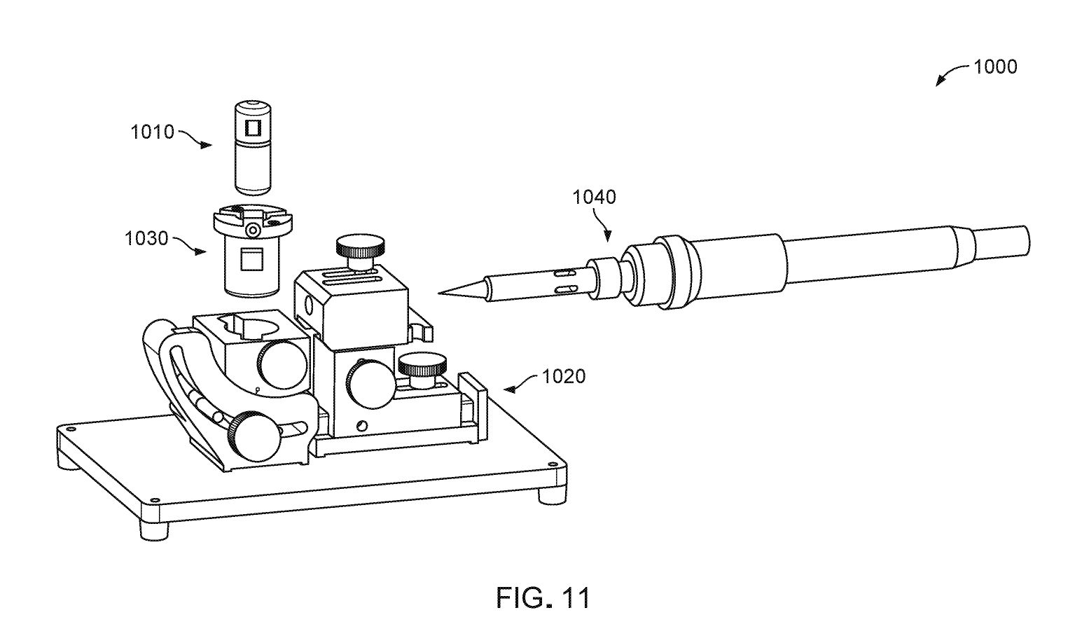

FIGS. 10 and 11 illustrate a system 1000 that can be used to pierce one or more holes in the side of an ingestible device 1010. System 1000 includes a tool 1020, a device holder 1030 and a soldering iron 1040.

In some embodiments, soldering iron tool 1020 includes primarily machined aluminum components. Tool 1020 is designed to assist in the production of two holes in the side of device 1010 through which sample contained within device 1010 is extracted. Tool 1020 has a keyed cradle which accepts device holder 1030. The cradle has two keying features which position holder 1030 and device 1010 into two distinct radial positions to produce the holes in device 1010 in the target locations in the side of each sampling chamber. A guide channel in tool 1020 accurately positions the tip of soldering iron 1040 in the correct position and at the correct depth to control the location and size of the melted holes in device 1010. Tool 1020 also has knobs to adjust the height, distance and penetration depth of soldering iron 1040 and to adjust the angle of device 1010 with respect to soldering iron 1040. Alternatively, or additionally, safety screws may be used to lock the positions of the parts of tool 1020, which can, for example, reduce the possibility of accidental misalignment during use of tool 1020. Optionally, soldering iron is operated manually 1040. However, in some embodiments, soldering iron 1040 is operated automatically. In certain embodiments, soldering iron 1040 is attached to tool 1020 such that movement of iron 1040 is automated with a motor to accurately control penetration depth, exposure time and applied force. In some embodiments, soldering iron 1040 is on a rail fixed to the tool 1020 and uses a spring loaded trigger mechanism to snap soldering iron 1040 back automatically once it has been pushed forward and reached the target.

Device holder 1030 can be made of machined polycarbonate. Holder 1030 holds device 1010 during the hole creation and centrifugation processes. In some embodiments, holder 1030 uses a set screw or the like (e.g., a ball detent, snap, spring loaded tab, or other keying feature) to lock device 1010 in place by keying into the window of device 1010. When positioned correctly, the set screw does not apply pressure to device 1010, but simply protrudes into the window of device 1010. Holder 1030 is placed into the cradle in soldering iron tool 1020. An external keying feature on the side of holder 1030 helps locate holder 1030 into one of two positions within tool 1020. The two positions ensure that the tip of soldering iron 1040 is guided into the two hole locations. After the holes in device 1010 have been produced, holder 1030 is removed from tool 1020 and is used to key device 1010 into a centrifuge jig which is then centrifuged to extract the samples (see discussion above).

In some embodiments, soldering iron 1040 is a hand-held tool that has a heatable tip. As an example, the tip can be heated electrically, and the temperature can be set using an interface of a control system of system 1000. The heatable tip can be a commercially available tip, or it can be custom made. After the tip of soldering iron 1040 is heated to the target temperature, the tip is inserted into a guide in soldering iron tool 1020. The guide aligns the tip to the correct location on device 1010 and restricts the depth to which soldering iron 1040 can be pushed. The tip is pushed into the device 1010 (e.g., polycarbonate housing) to produce the holes. The heat of the tip melts the polycarbonate, allowing for penetration while minimizing the applied force. Optionally, the tip can be a single use tip.

While system 1000 has been described with a soldering iron, other embodiments are possible. For example, additionally or alternatively, system 1000 can use one or more heated lancets (see discussion above). In some embodiments, soldering iron 1040 can be replaced by rotary cutting tool, such as a dremel, to drill holes into capsules. In certain embodiments of a fully automated system, soldering iron 1040 can be replaced by custom heated tips that could be provided, for example, in cartridges which are attached to tool 1000. In such embodiments, tool 1000 can be configured to dispense the tips, heat the tips (e.g., using an onboard electrical system), move one tip forward at a time to melt the holes in the capsule, and eject the tip. Optionally, tips could then be discarded. In some embodiments, the number of components in system 1000 can be simplified to reduce the number of adjustment options. Alternatively, system 1000 could be configured so that there are more adjustment options. In certain embodiments, components of system 1000 can be adjusted by adding rails and lead screws or rack and pinion systems to allow positions of components to be adjusted more precisely.

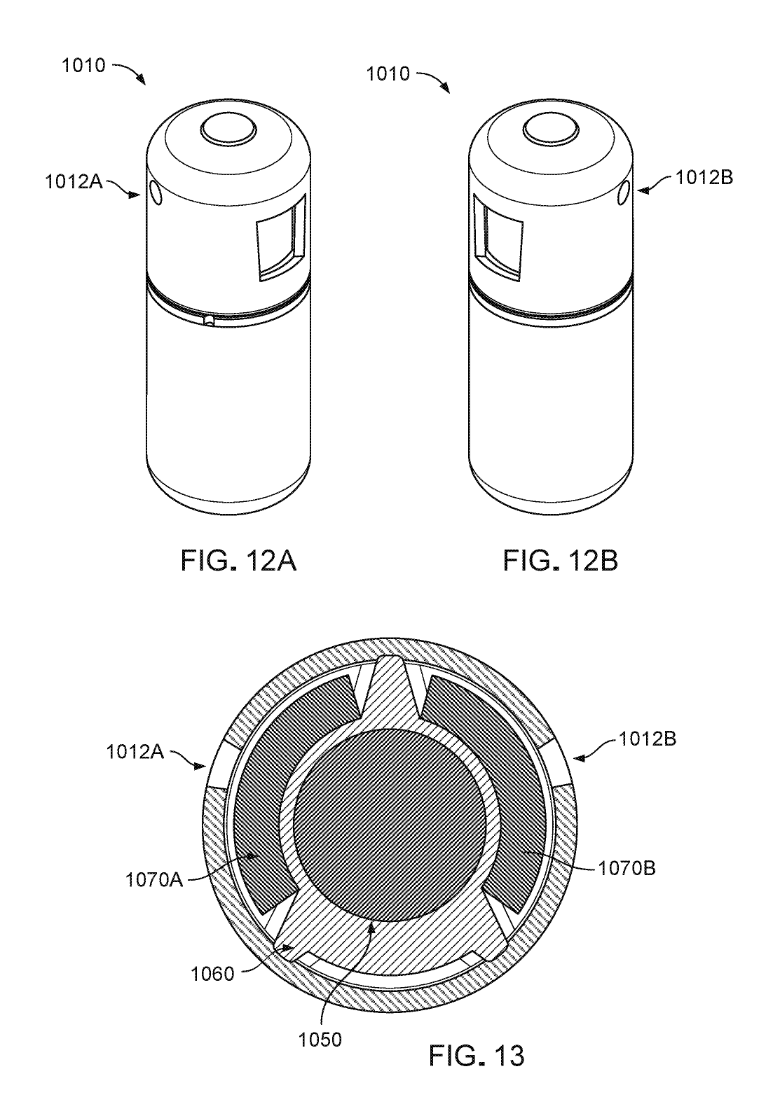

FIGS. 12A and 12B illustrate device 1010 with holes 1012A and 1012B formed, for example, using the approach noted above. In general holes 1012A and 1012B are centered on corresponding, respective sampling chamber of device 1010. Thus, in some embodiments 1012A and 1012B are not separated from each other by 180°. The location of holes 1012A and 1012B allows sample to exit the sample chambers when device 1010 is centrifuged upside down with holes facing away from the centrifuge's rotor.

FIG. 13 illustrates a cross-sectional view of an embodiment of device 1010 with holes 1012A and 1012B. Device 1010 includes a gearmotor 1050 and a wiper 1060. A shaft of gearmotor 1050 is connected to the top cap of device 1010 so that the activation of gearmotor 1050 spins the top cap of device 1010. This in turn allows the sampling window in the top cap to be rotated so that it aligns with the sampling chambers formed by wiper 1060. Wiper 1060 contacts the internal surface of device 1010 and forms part of the boundaries between holes 1012A and 1012B. Wiper 1060 may define a liquid tight barrier that isolates a sample in chamber 1012A from a sample in 1012B. As shown in FIG. 13, device 1010 may further include absorbent material members 1070A and 1070B. Members 1070A and 1070B may be, for example, sponges (e.g., hydrophilic sponges, hydrophobic sponges). Members 1070A and 1070B can assist in chambers 1012A and 1012B collecting and maintaining their respective samples.

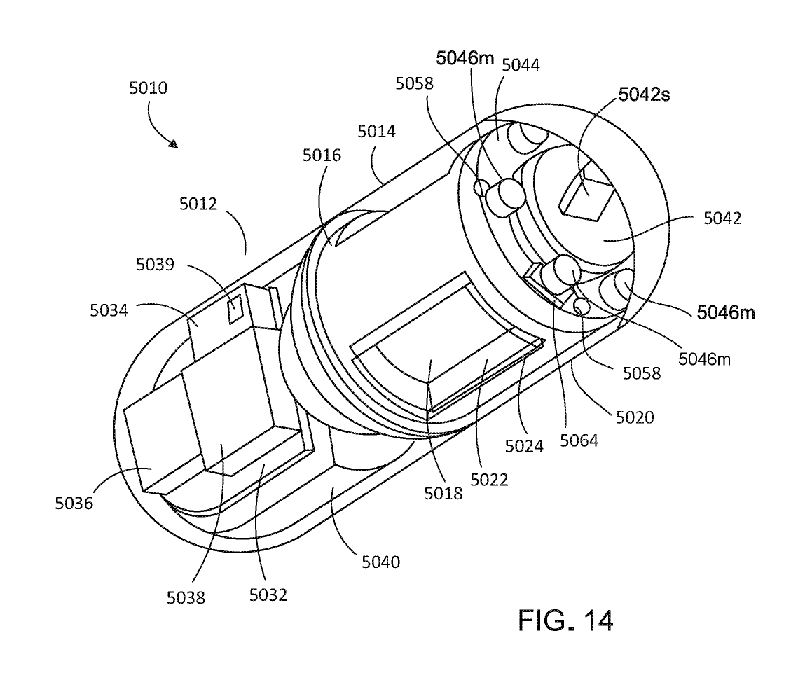

FIG. 14 shows a partial view of an exemplary embodiment of an ingestible device 5010 in which a portion of the enclosure of ingestible device 5010 has been removed. Ingestible device 5010 may be used for collecting substances. Ingestible device 5010 may generally be in the shape of a capsule, like a conventional pill. Accordingly, the shape of ingestible device 5010 provides for easier ingestion and is also familiar to healthcare practitioners and patients.

The structure of ingestible device 5010 includes first portions and second portions 5012 and 5014. First portion 5012 includes control electronics, a power supply, and a communication system. Second portion 5014 is generally configured to interact with the GI tract, such as, for example but not limited to, sample collection, substance delivery and environmental monitoring. Second portion 5014 includes a storage sub-unit 16 with one or more chambers 5018 and a chamber enclosure 5020 that encloses or overlays a storage sub-unit 5016. Each chamber 5018 has a corresponding chamber opening 5022. Chamber enclosure 5020 has an access port 5024. In this example embodiment, ingestible device 5010 includes three chambers 5018, but there can be other embodiments that have one, two or more than three chambers 5018.

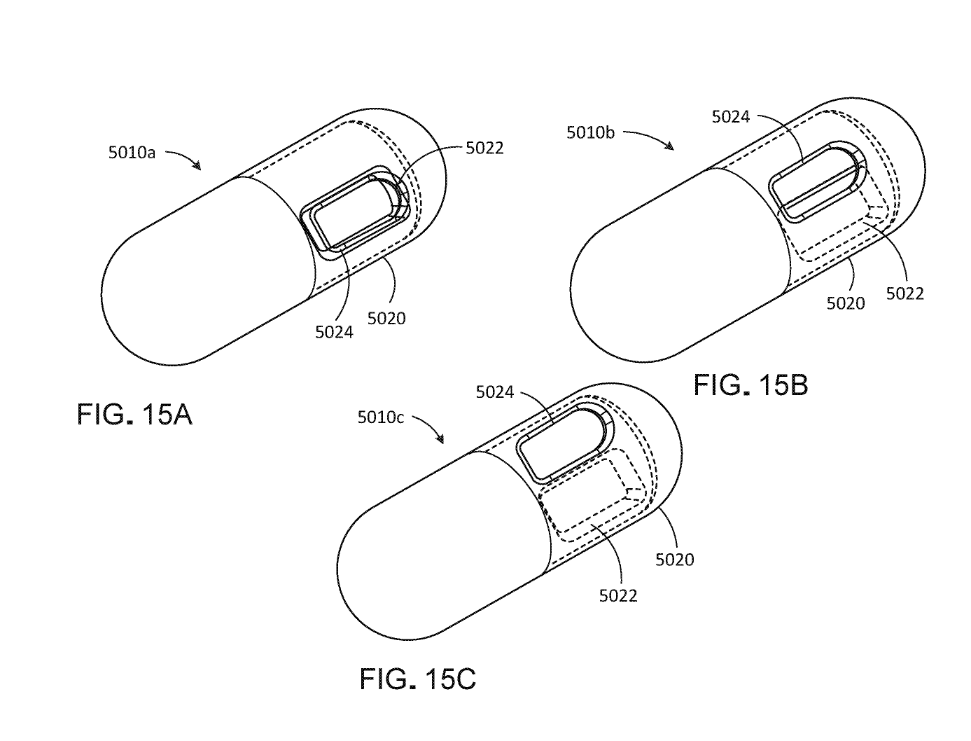

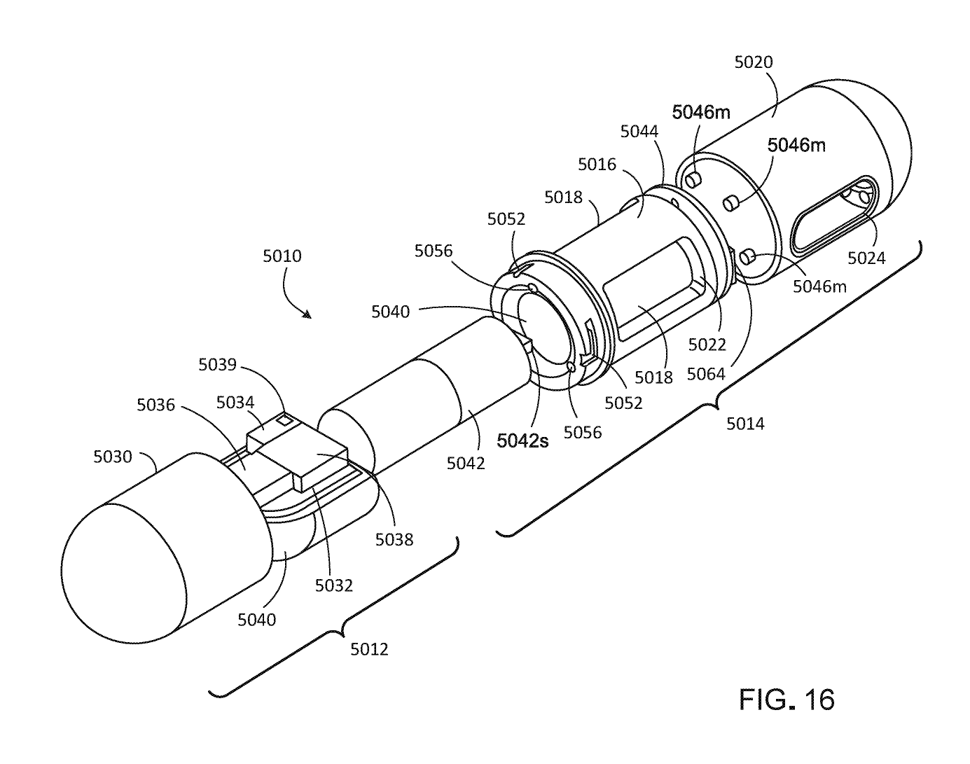

FIGS. 15A-15C illustrate operation of ingestible device 5010. Generally, chamber enclosure 5020 operates as a “closed-loop” revolver mechanism. Chamber enclosure 5020 rotates, in a controlled manner, to align the access port 5024 with each of chamber openings 5022 for collecting, at targeted locations, samples of the contents in the GI into corresponding chamber 5018, and/or for delivering substances stored in chambers 5018 to targeted locations within the body.

Generally, during collection of samples, the rotation of chamber enclosure 5020 may be described as a “closed-loop” revolver mechanism because each chamber opening 5022 is exposed only once during the passage of ingestible device 5010 within the body in order to avoid cross-contamination of the collected samples. In other words, in some embodiments, chamber enclosure 5020 ideally rotates only once when collecting samples during each usage of ingestible device 5010 so that access port 5024 aligns with each of chamber openings 5022 serially and only once. That is, during collection of samples, access port 2224 does not bypass any chamber opening 5022 and also does not return to a previous chamber opening 5022 during its rotation.

In some embodiments, chamber enclosure 5020 can rotate in a bidirectional motion before completing one revolution and/or perform multiple revolutions during one usage of the ingestible device 5010 so that at least one chamber opening 5022 is exposed multiple times. A chamber opening 5022 may need to be exposed multiple times if its corresponding chamber stores solids or semi-solid reagents, sensors or cleaning agents for cleaning the GI tract.

As illustrated in FIG. 15A, shown therein generally is ingestible device 5010 in an open position 5010a in which access port 5024 on chamber enclosure 5020 is aligned with a chamber opening 5022. In this configuration, ingestible device 5010 may collect substances through chamber opening 5022. In other words, the contents of the GI tract may be forced into exposed chamber 5018 through muscular contractions (e.g., peristalsis).

Thereafter, chamber enclosure 5020 may rotate to seal chamber opening 5022. FIG. 15B shows ingestible device 5010 with a partially open/partially closed position 5010b in which access port 5024 has been rotated such that chamber enclosure 5020 partially seals chamber opening 5022.

FIG. 15C shows ingestible device 5010 in a closed position 5010c, in which the chamber enclosure 5020 has been rotated a distance such that access port 5024 completely seals chamber opening 5022. If chamber enclosure 5020 has not rotated one revolution, chamber enclosure 5020 may continue to rotate in the same direction in order to align access port 5024 with another chamber opening 5022 depending if ingestible device 5010 has been configured to perform another operation (i.e. sampling or distribution).

In another example embodiment, chamber enclosure 5020 may be stationary and storage sub-unit 5016 may instead rotate to align its one or more chamber openings 5022 with access port 5024. Rotating storage sub-unit 5016 instead of chamber enclosure 5020 may provide greater control over the rotation motion and a more constant motion since storage sub-unit 5016 would not be subjected to a varying viscosity arising from the contents in the GI tract. This arrangement, however, may limit a volume of at least one of chambers 5018.

In some embodiments, chamber enclosure 5020 or storage sub-unit 5016 may rotate in a predetermined sequence of bidirectional rotational motions. As described above, when storage sub-unit 5016 is configured to rotate instead of chamber enclosure 5020, the volume of at least one of chambers 5018 can be limited. In order to avoid having to limit the volume of the chambers 5018, non-recess areas that may be used to separate different chambers 5018 in storage sub-unit 5016 may be minimized in volume or removed. Ingestible device 5010 can rotate in a first direction for aligning access port 5024 with one of the two adjacent chambers. Ingestible device 5010 can be configured to rotate in a second direction that is opposite to the first direction in order to avoid cross contamination between samples collected into or substances released from those two adjacent chambers.

Ingestible device 5010 may be used for collecting usable samples from the contents of the GI tract (e.g., 100 μL sized samples) and maintaining each sample in isolation from one another until the samples are extracted.

In some embodiments, ingestible device 5010 may also be configured to conduct in-vivo measurements. Ingestible device 5010 is introduced into the body with some of chambers 5018 being empty and some of chambers 5018 carrying at least one reagents. At a predefined location in the body, ingestible device 5010 is configured to collect a sample from the GI tract and to store the sample into a chamber carrying at least one reagent. After collection, in-vivo analysis may be conducted based on how the collected sample interacts with the reagent inside chamber 5018. For example, ingestible device 5010 may use a biochemistry assay, such as an enzyme-linked immunosorbent assay (ELISA), for performing in-situ experiments on collected samples. Alternatively, peripherals can be included into chambers 5018 for changing the dynamics of several in-vivo analysis and measurements. The peripherals may include a light source, a receiver, a transducer, a heater, and the like. In general, the in-vivo experiments vary according to the type of information that is being sought.

FIG. 16 illustrates an exploded view of the components of ingestible device 5010 in one example embodiment. First portion 5012 of ingestible device 5010 includes an end closure 5030, and electronic components embedded on a main printed circuit board (PCB) 5032 including a communication subsystem having communication peripherals 5034 and a transceiver 5036, a main microcontroller (i.e. processor) 5038, a power supply 5040 and other peripheral components described in further detail below. Second portion 5014 of ingestible device 5010 generally includes a motor 5042, storage sub-unit 5016, a secondary PCB 5044, an encoding magnet arrangement 5046m and the chamber enclosure 5020. Generally, by placing main PCB 5032 and secondary PCB 5044 in distinct regions inside ingestible device 5010, they may be prevented from experiencing the same electrical or physical hazards. Motor 42 is inserted into a motor compartment 5054 that is located in the center of storage sub-unit 5016. PCB 5044 is annular and includes one or more peripheral electronic components (e.g., a capacitor 5062 and a resistor 4060, which can be used as a pull-up resistor), and a sensor 5064. 5039 is a magnetic switch. 5042s is a shaft. 5056 are access holes.

End enclosure 5030 provides a hollow space defined by an inner wall 5048 that is cylindrical with a domed end portion. End enclosure 5030 also includes engagement members 5050 for aligning and releasably engaging with storage sub-unit 5016 to releasably lock end enclosure 5030 in place during operation. In particular, engagement members 5050 releasably engage complementary structures 5052 in storage sub-unit 5016. When end enclosure 5030 locks with storage sub-unit 5016, end enclosure 5030 overlaps with a rear of storage sub-unit 5016 and creates a seal. In some embodiments, the overlap between end enclosure 5030 and storage sub-unit 5016 may span a width of 3 millimeters.

Some or all of the sponges of the above-described sampling systems may contain one or more preservatives. Typically, the assay sponge and/or the volume sponge 1230 and/or the transfer sponge contain one or more preservatives. Typically, the preservative(s) are selected based on the analyte of interest, e.g., an analyte (such as a nucleic acid or protein biomarker) for a GI disorder.

Typically, the ingestible devices disclosed herein include one or more processing devices, and one more machine readable hardware storage devices. In some embodiments, the one or more machine readable hardware storage devices store instructions that are executable by the one or more processing devices to determine the location of the ingestible device in a portion of a GI tract of the subject. In certain embodiments, the one or more machine readable hardware storage devices store instructions that are executable by the one or more processing devices to transmit data to an external device (e.g., a base station external to the subject, such as a base station carried on an article worn by the subject) capable of implementing the data to determine the location of the device within the GI tract of the subject.

In some embodiments, the location of the ingestible device within the GI tract of the subject can be determined to an accuracy of at least 85%, e.g., at least 90%, at least 95%, at least 97%, at least 98%, at least 99%, 100%. In such embodiments, the portion of the portion of the GI tract of the subject can include, for example, the esophagus, the stomach, duodenum, the jejunum, and/or the terminal ileum, cecum and colon.

In certain embodiments, the location of the ingestible device within the esophagus of the subject can be determined to an accuracy of at least 85%, e.g., at least 90%, at least 95%, at least 97%, at least 98%, at least 99%, 100%.

In some embodiments, the location of the ingestible device within the stomach of the subject can be determined to an accuracy of at least 85%, e.g., at least 90%, at least 95%, at least 97%, at least 98%, at least 99%, 100%.

In certain embodiments, the location of the ingestible device within the duodenum of the subject can be determined to an accuracy of at least 85%, e.g., at least 90%, at least 95%, at least 97%, at least 98%, at least 99%, 100%.

In some embodiments, the location of the ingestible device within the jejunum of the subject can be determined to an accuracy of at least 85%, e.g., at least 90%, at least 95%, at least 97%, at least 98%, at least 99%, 100%.

In certain embodiments, the location of the ingestible device within the terminal ileum, cecum and colon of the subject can be determined to an accuracy of at least 85%, e.g., at least 90%, at least 95%, at least 97%, at least 98%, at least 99%, 100%.

In some embodiments, the location of the ingestible device within the cecum of the subject can be determined to an accuracy of at least 85%, e.g., at least 90%, at least 95%, at least 97%, at least 98%, at least 99%, 100%.

As used herein, the term “reflectance” refers to a value derived from light emitted by the device, reflected back to the device, and received by a detector in or on the device. For example, in some embodiments this refers to light emitted by the device, wherein a portion of the light is reflected by a surface external to the device, and the light is received by a detector located in or on the device.

As used herein, the term “illumination” refers to any electromagnetic emission. In some embodiments, an illumination may be within the range of Infrared Light (IR), the visible spectrum and ultraviolet light (UV), and an illumination may have a majority of its power centered at a particular wavelength in the range of 100 nm to 1000 nm. In some embodiments, it may be advantageous to use an illumination with a majority of its power limited to one of the infrared (750 nm-1000 nm), red (600 nm-750 nm), green (495 nm-600 nm), blue (400 nm-495 nm), or ultraviolet (100 nm-400 nm) spectrums. In some embodiments a plurality of illuminations with different wavelengths may be used. For illustrative purposes, the embodiments described herein may refer to the use of green or blue spectrums of light. However, it is understood that these embodiments may use any suitable light having a wavelength that is substantially or approximately within the green or blue spectra defined above, and the localization systems and methods described herein may use any suitable spectra of light.

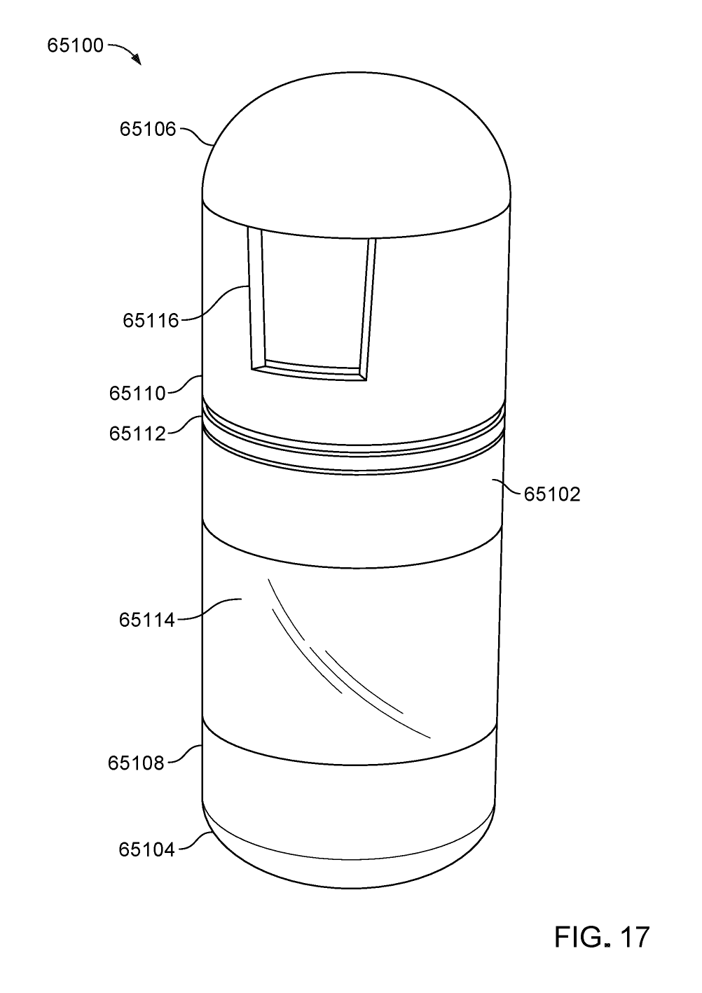

Referring now to FIG. 17, shown therein is a view of an example embodiment of an ingestible device 65100, which may be used to identify a location within a gastrointestinal (GI) tract. It is to be understood that certain details regarding the design of ingestible device 65100 are not shown in FIG. 17 and the following figures, and that, in general, various aspect of ingestible devices described elsewhere herein can be implemented in ingestible device 65100 and the ingestible devices shown in the following figures.

In some embodiments, ingestible device 65100 may be configured to autonomously determine whether it is located in the stomach, a particular portion of the small intestine such as a duodenum, jejunum, or ileum, or the large intestine by utilizing sensors operating with different wavelengths of light. Additionally, ingestible device 65100 may be configured to autonomously determine whether it is located within certain portions of the small intestine or large intestine, such as the duodenum, the jejunum, the cecum, or the colon.

Ingestible device 65100 may have a housing 65102 shaped similar to a pill or capsule. The housing 65102 of ingestible device 65100 may have a first end portion 65104, and a second end portion 65106. The first end portion 65104 may include a first wall portion 65108, and second end portion 65106 may include a second wall portion 65110. In some embodiments, first end portion 65104 and second end portion 65106 of ingestible device 65100 may be manufactured separately, and may be affixed together by a connecting portion 65112.

In some embodiments, ingestible device 65100 may include an optically transparent window 65114. Optically transparent window 65114 may be transparent to various types of illumination in the visible spectrum, infrared spectrum, or ultraviolet light spectrum, and ingestible device 65100 may have various sensors and illuminators located within the housing 65102, and behind the transparent window 65114. This may allow ingestible device 65100 to be configured to transmit illumination at different wavelengths through transparent window 65114 to an environment external to housing 65102 of ingestible device 65100, and to detect a reflectance from a portion of the illumination that is reflected back through transparent window 65114 from the environment external to housing 65102. Ingestible device 65100 may then use the detected level of reflectance in order to determine a location of ingestible device 65100 within a GI tract. In some embodiments, optically transparent window 65114 may be of any shape and size, and may wrap around the circumference of ingestible device 65100. In this case, ingestible device 65100 may have multiple sets of sensors and illuminators positioned at different locations azimuthally behind window 65114.

In some embodiments, ingestible device 65100 may optionally include an opening 65116 in the second wall portion 65110. In some embodiments, the second wall portion 65110 may be configured to rotate around the longitudinal axis of ingestible device 65100 (e.g., by means of a suitable motor or other actuator housed within ingestible device 65100). This may allow ingestible device 65100 to obtain a fluid sample from the GI tract, or release a substance into the GI tract, through opening 65116.

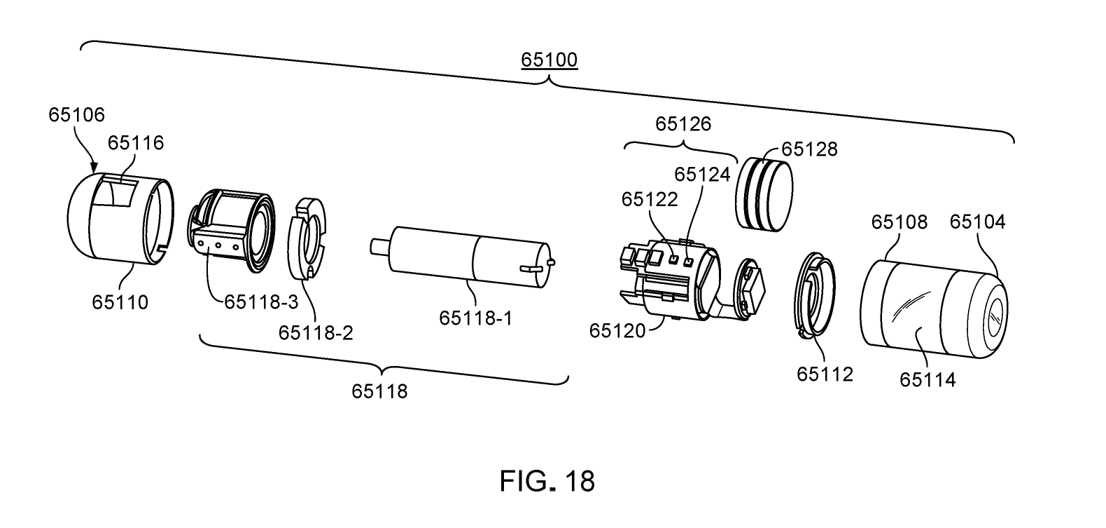

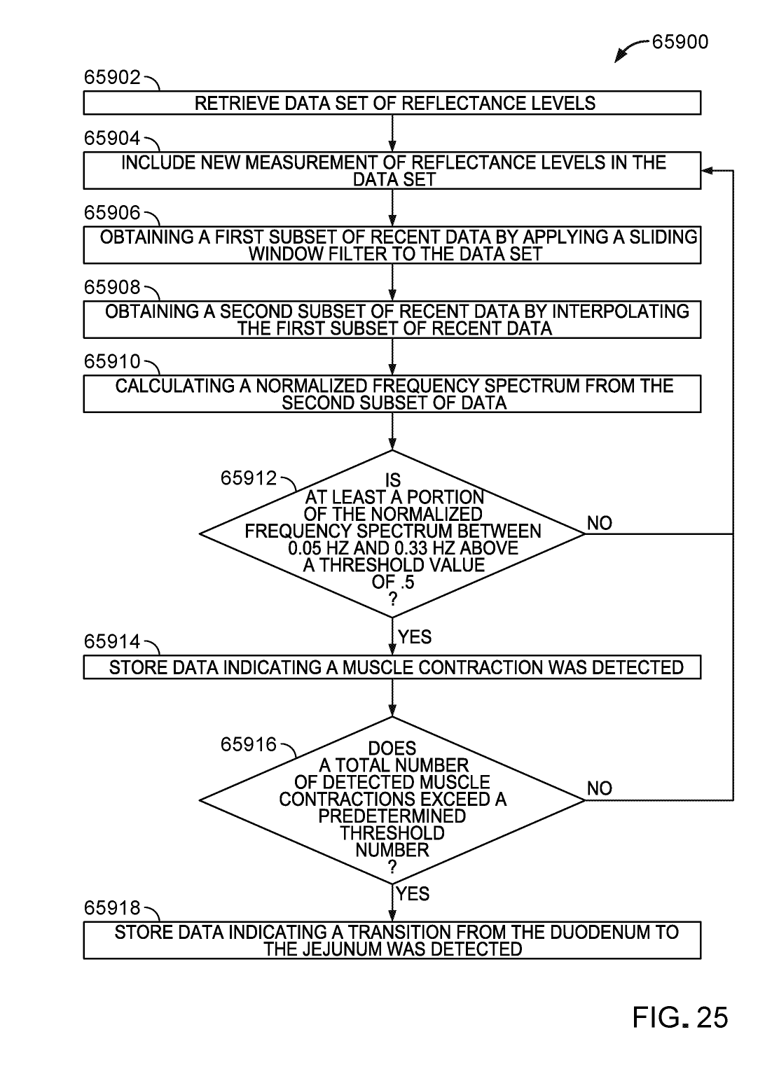

FIG. 18 shows an exploded view of ingestible device 65100. In some embodiments, ingestible device 65100 may optionally include a rotation assembly 65118. Optional rotation assembly 65118 may include a motor 65118-1 driven by a microcontroller (e.g., a microcontroller coupled to printed circuit board 65120), a rotation position sensing ring 65118-2, and a storage sub-unit 65118-3 configured to fit snugly within the second end portion 65104. In some embodiments, rotation assembly 65118 may cause second end portion 65104, and opening 65116, to rotate relative to the storage sub-unit 65118-3. In some embodiments, there may be cavities on the side of storage sub-unit 65118-3 that function as storage chambers. When the opening 65116 is aligned with a cavity on the side of the storage sub-unit 65118-3, the cavity on the side of the storage sub-unit 65118-3 may be exposed to the environment external to the housing 65102 of ingestible device 65100. In some embodiments, the storage sub-unit 65118-3 may be loaded with a medicament or other substance prior to the ingestible device 65100 being administered to a subject. In this case, the medicament or other substance may be released from the ingestible device 65100 by aligning opening 65116 with the cavity within storage sub-unit 65118-3. In some embodiments, the storage sub-unit 65118-3 may be configured to hold a fluid sample obtained from the GI tract. For example, ingestible device 65100 may be configured to align opening 65116 with the cavity within storage sub-unit 65118-3, thus allowing a fluid sample from the GI tract to enter the cavity within storage sub-unit 65118-3. Afterwards, ingestible device 65100 may be configured to seal the fluid sample within storage sub-unit 65118-3 by further rotating the second end portion 65106 relative to storage sub-unit 65118-3. In some embodiments, storage sub-unit 118-3 may also contain a hydrophilic sponge, which may enable ingestible device 65100 to better draw certain types of fluid samples into ingestible device 65100. In some embodiments, ingestible device 65100 may be configured to either obtain a sample from within the GI tract, or to release a substance into the GI tract, in response to determining that ingestible device 65100 has reached a predetermined location within the GI tract. For example, ingestible device 65100 may be configured to obtain a fluid sample from the GI tract in response to determining that the ingestible device has entered the jejunum portion of the small intestine (e.g., as determined by process 65900 discussed in relation to FIG. 25). Other ingestible devices capable of obtaining samples or releasing substances are discussed in commonly-assigned PCT Application No. PCT/CA2013/000133 filed Feb. 15, 2013, commonly-assigned U.S. Provisional Application No. 62/385,553, and commonly-assigned U.S. Provisional Application No. 62/376,688, which each are hereby incorporated by reference herein in their entirety. It is understood that any suitable method of obtaining samples or releasing substances may be incorporated into some of the embodiments of the ingestible devices disclosed herein, and that the systems and methods for determining a location of an ingestible device may be incorporated into any suitable type of ingestible device.

Ingestible device 65100 may include a printed circuit board (PCB) 65120, and a battery 65128 configured to power PCB 65120. PCB 65120 may include a programmable microcontroller, and control and memory circuitry for holding and executing firmware or software for coordinating the operation of ingestible device 65100, and the various components of ingestible device 65100. For example, PCB 65120 may include memory circuitry for storing data, such as data sets of measurements collected by sensing sub-unit 65126, or instructions to be executed by control circuitry to implement a localization process, such as, for example, one or more of the processes, discussed herein, including those discussed below in connection with one or more of the associated flow charts. PCB 65120 may include a detector 65122 and an illuminator 65124, which together form sensing sub-unit 65126. In some embodiments, control circuitry within PCB 65120 may include processing units, communication circuitry, or any other suitable type of circuitry for operating ingestible device 65100. For illustrative purposes, only a single detector 65122 and a single illuminator 65124 forming a single sensing sub-unit 65126 are shown. However, it is understood that in some embodiments there may be multiple sensing sub-units, each with a separate illuminator and detector, within ingestible device 65100. For example, there may be several sensing sub-units spaced azimuthally around the circumference of the PCB 65120, which may enable ingestible device 65100 to transmit illumination and detect reflectances or ambient light in all directions around the circumference of the device. In some embodiments, sensing sub-unit 65126 may be configured to generate an illumination using illuminator 65124, which is directed through the window 65114 in a radial direction away from ingestible device 65100. This illumination may reflect off of the environment external to ingestible device 65100, and the reflected light coming back into ingestible device 65100 through window 65114 may be detected as a reflectance by detector 65122.

In some embodiments, window 65114 may be of any suitable shape and size. For example, window 65114 may extend around a full circumference of ingestible device 65100. In some embodiments there may be a plurality of sensing sub-units (e.g., similar to sensing sub-unit 65126) located at different positions behind the window. For example, three sensing sub-units may be positioned behind the window at the same longitudinal location, but spaced 120 degrees apart azimuthally. This may enable ingestible device 65100 to transmit illuminations in all directions radially around ingestible device 65100, and to measure each of the corresponding reflectances.

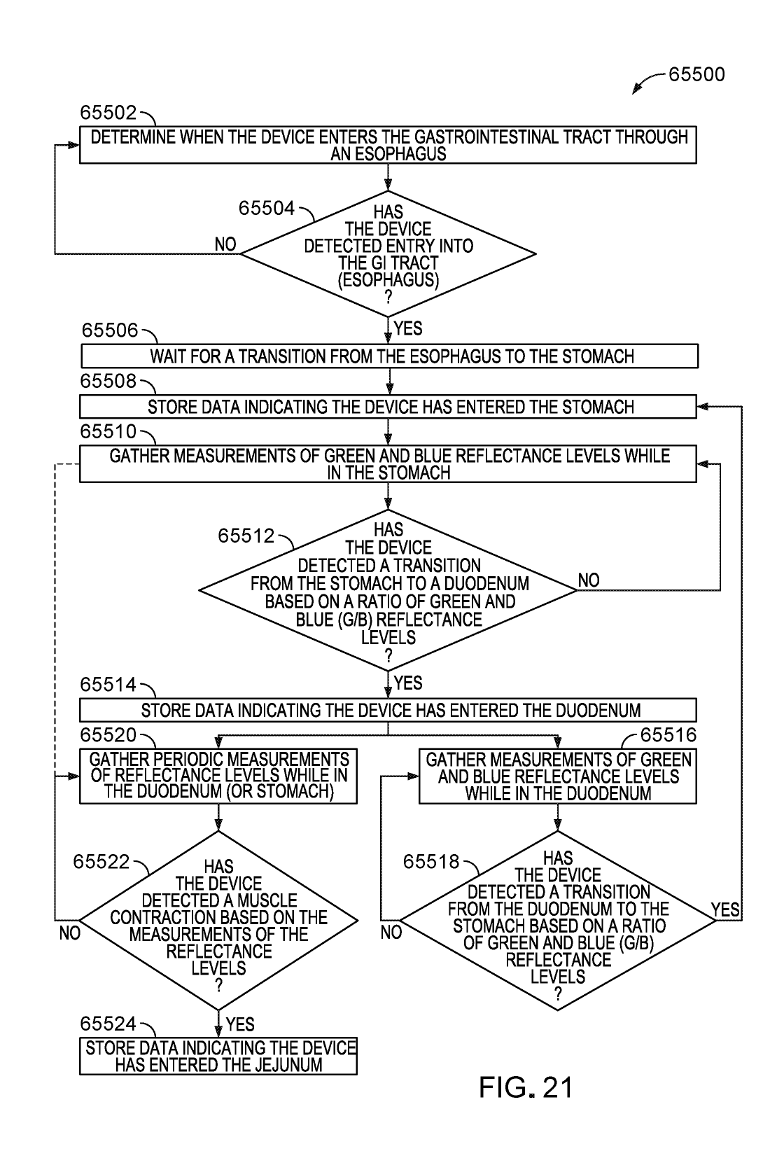

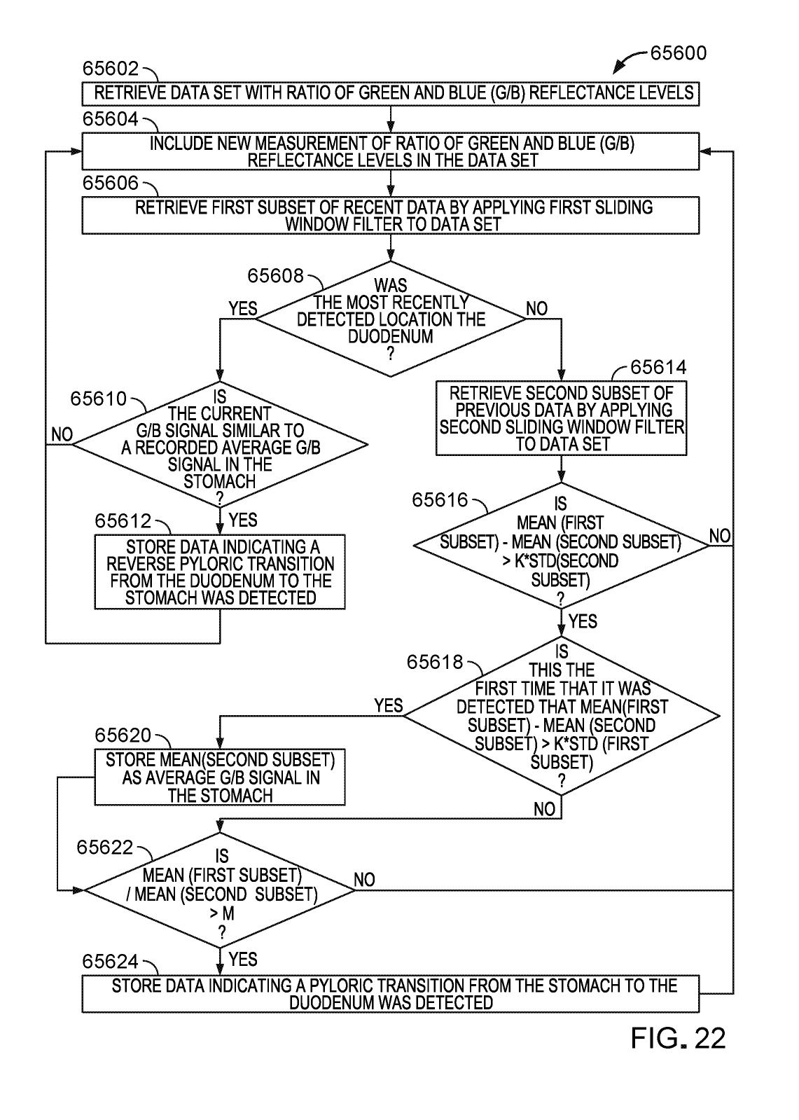

In some embodiments, illuminator 65124 may be capable of producing illumination at a variety of different wavelengths in the ultraviolet, infrared, or visible spectrum. For example, illuminator 65124 may be implemented by using Red-Green-Blue Light-Emitting diode packages (RGB-LED). These types of RGB-LED packages are able to transmit red, blue, or green illumination, or combinations of red, blue, or green illumination. Similarly, detector 65122 may be configured to sense reflected light of the same wavelengths as the illumination produced by illuminator 65124. For example, if illuminator 65124 is configured to produce red, blue, or green illumination, detector 65122 may be configured to detect different reflectances produced by red, blue, or green illumination (e.g., through the use of an appropriately configured photodiode). These detected reflectances may be stored by ingestible device 65100 (e.g., within memory circuitry of PCB 65120), and may then be used by ingestible device 65100 in determining a location of ingestible device 65100 within the GI tract (e.g., through the use of process 65500 (FIG. 21), process 65600 (FIG. 22), or process 65900 (FIG. 25)).

It is understood that ingestible device 65100 is intended to be illustrative, and not limiting. It will be understood that modifications to the general shape and structure of the various devices and mechanisms described in relation to FIG. 17 and FIG. 18 may be made without significantly changing the functions and operations of the devices and mechanisms. For example, ingestible device 65100 may have a housing formed from a single piece of molded plastic, rather than being divided into a first end portion 65104 and a second end portion 65106. As an alternate example, the location of window 65114 within ingestible device 65100 may be moved to some other location, such as the center of ingestible device 65100, or to one of the ends of ingestible device 65100. Moreover, the systems and methods discussed in relation to FIGS. 65-74 may be implemented on any suitable type of ingestible device, provided that the ingestible device is capable of detecting reflectances or levels of illumination in some capacity. For example, in some embodiments ingestible device 65100 may be modified to replace detector 65122 with an image sensor, and the ingestible device may be configured to measure relative levels of red, blue, or green light by decomposing a recorded image into its individual spectral components. Other examples of ingestible devices with localization capabilities, which may be utilized in order to implement the systems and methods discussed in relation to FIG. 17-27, are discussed in co-owned PCT Application No. PCT/US2015/052500 filed on Sep. 25, 2015, which is hereby incorporated by reference herein in its entirety. Furthermore, it should be noted that the features and limitations described in any one embodiment may be applied to any other embodiment herein, and the descriptions and examples relating to one embodiment may be combined with any other embodiment in a suitable manner.

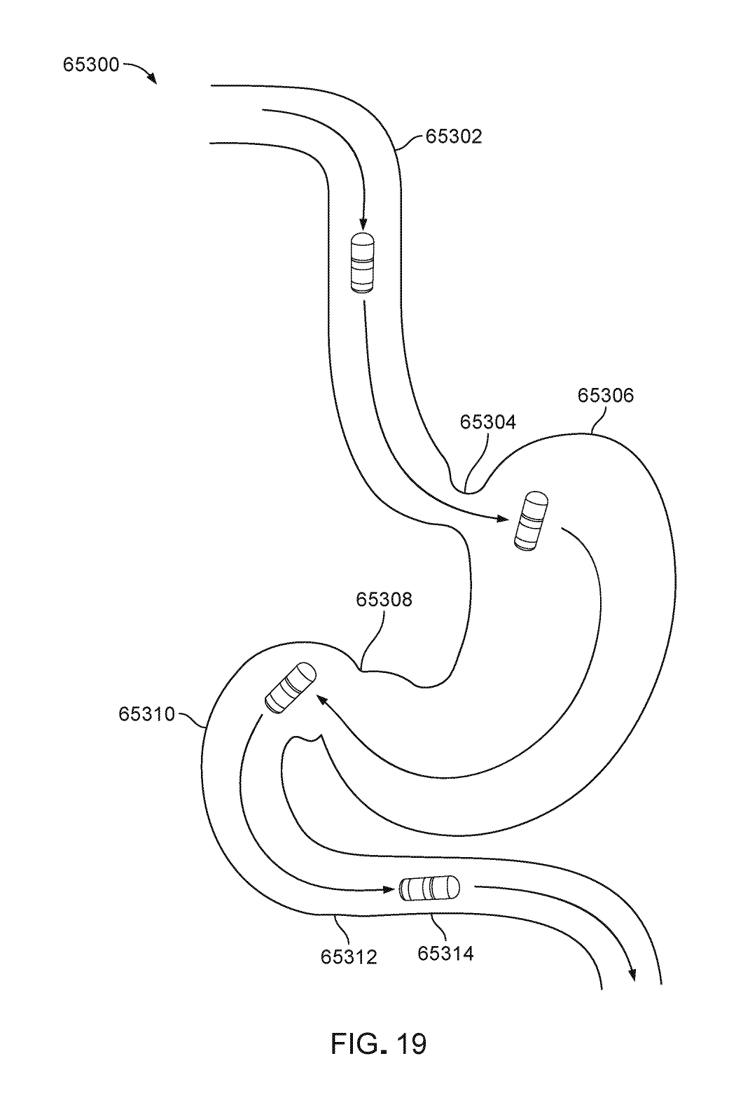

FIG. 19 is a diagram of an ingestible device during an example transit through a gastrointestinal (GI) tract, in accordance with some embodiments of the disclosure. Ingestible device 65300 may include any portion of any other ingestible device discussed in this disclosure (e.g., ingestible device 65100 (FIG. 17)), and may be any suitable type of ingestible device with localization capabilities. For example, ingestible device 65300 may be one embodiment of ingestible device 65100 without the optional opening 65116 (FIG. 17) or optional rotation assembly 65118 (FIG. 18)). In some embodiments, ingestible device 65300 may be ingested by a subject, and as ingestible device 65300 traverses the GI tract, ingestible device 65300 may be configured to determine its location within the GI tract. For example, the movement of ingestible device 65300 and the amount of light detected by ingestible device 65300 (e.g., via detector 65122 (FIG. 18)) may vary substantially depending on the location of ingestible device 65300 within the GI tract, and ingestible device 65300 may be configured to use this information to determine a location of ingestible device 65300 within the GI tract. For instance, ingestible device 65300 may detect ambient light from the surrounding environment, or reflectances based on illumination generated by ingestible device 65300 (e.g., generated by illuminator 65124 (FIG. 17)), and use this information to determine a location of ingestible device 65300 through processes, such as described herein. The current location of ingestible device 65300, and the time that ingestible device 65300 detected each transition between the various portions of the GI tract, may then be stored by ingestible device 65300 (e.g., in memory circuitry of PCB 65120 (FIG. 18)), and may be used for any suitable purpose.

Shortly after ingestible device 65300 is ingested, ingestible device will traverse the esophagus 65302, which may connect the subject's mouth to a stomach 65306. In some embodiments, ingestible device 65300 may be configured to determine that it has entered the esophagus portion GI tract by measuring the amount and type of light (e.g., via detector 65122 (FIG. 18)) in the environment surrounding the ingestible device 65300. For instance, ingestible device 65300 may detect higher levels of light in the visible spectrum (e.g., via detector 65122 (FIG. 18)) while outside the subject's body, as compared to the levels of light detected while within the GI tract. In some embodiments, ingestible device 65300 may have previously stored data (e.g., on memory circuitry of PCB 65120 (FIG. 18)) indicating a typical level of light detected when outside of the body, and the ingestible device 65300 may be configured to determine that entry to the body has occurred when a detected level of light (e.g., detected via detector 65122 (FIG. 18)) has been reduced beyond a threshold level (e.g., at least a 20-30% reduction) for a sufficient period of time (e.g., 5.0 seconds).

In some embodiments, ingestible device 65300 may be configured to detect a transition from esophagus 65302 to stomach 65306 by passing through sphincter 65304. In some embodiments, ingestible device 65300 may be configured to determine whether it has entered stomach 65306 based at least in part on a plurality of parameters, such as but not limited to the use of light or temperature measurements (e.g., via detector 65122 (FIG. 18) or via a thermometer within ingestible device 65300), pH measurements (e.g., via a pH meter within ingestible device 65300), time measurements (e.g., as detected through the use of clock circuitry included within PCB 65120 (FIG. 18)), or any other suitable information. For instance, ingestible device 65300 may be configured to determine that ingestible device 65300 has entered stomach 65306 after detecting that a measured temperature of ingestible device 65300 exceeds 31 degrees Celsius. Additionally, or alternately, ingestible device 65300 may be configured to automatically determine it has entered stomach 65306 after one minute (or another pre-set time duration parameter, 80 seconds, 90 seconds, etc.) has elapsed from the time that ingestible device 65300 was ingested, or one minute (or another pre-set time duration parameter, 80 seconds, 90 seconds, etc.) from the time that ingestible device 65300 detected that it has entered the GI tract.

Stomach 65306 is a relatively large, open, and cavernous organ, and therefore ingestible device 65300 may have a relatively large range of motion. By comparison, the motion of ingestible device 65300 is relatively restricted within the tube-like structure of the duodenum 65310, the jejunum 65314, and the ileum (not shown), all of which collectively form the small intestine. Additionally, the interior of stomach 65306 has distinct optical properties from duodenum 65310 and jejunum 65314, which may enable ingestible device 65300 to detect a transition from stomach 65306 to duodenum 65310 through the appropriate use of measured reflectances (e.g., through the use of reflectances measured by detector 65122 (FIG. 18)), as used in conjunction with process 65600 (FIG. 22)).

In some embodiments, ingestible device 65300 may be configured to detect a pyloric transition from stomach 65306 to duodenum 65310 through the pylorus 65308. For instance, in some embodiments, ingestible device 65300 may be configured to periodically generate illumination in the green and blue wavelengths (e.g., via illuminator 65124 (FIG. 18)), and measure the resulting reflectances (e.g., via detector 65122 (FIG. 18)). Ingestible device 65300 may be configured to then use a ratio of the detected green reflectance to the detected blue reflectance to determine whether ingestible device 65300 is located within the stomach 65306, or duodenum 65310 (e.g., via process 65600 (FIG. 22)). In turn, this may enable ingestible device 65300 to detect a pyloric transition from stomach 65306 to duodenum 65310, an example of which is discussed in relation to FIG. 22.

Similarly, in some embodiments, ingestible device 65300 may be configured to detect a reverse pyloric transition from duodenum 65310 to stomach 65306. Ingestible device 65300 will typically transition naturally from stomach 65306 to duodenum 65310, and onward to jejunum 65314 and the remainder of the GI tract. However, similar to other ingested substances, ingestible device 65300 may occasionally transition from duodenum 65310 back to stomach 65306 as a result of motion of the subject, or due to the natural behavior of the organs with the GI tract. To accommodate this possibility, ingestible device 65300 may be configured to continue to periodically generate illumination in the green and blue wavelengths (e.g., via illuminator 65124 (FIG. 18)), and measure the resulting reflectances (e.g., via detector 65122 (FIG. 18)) to detect whether or not ingestible device 65300 has returned to stomach 65306. An exemplary detection process is described in additional detail in relation to FIG. 22.

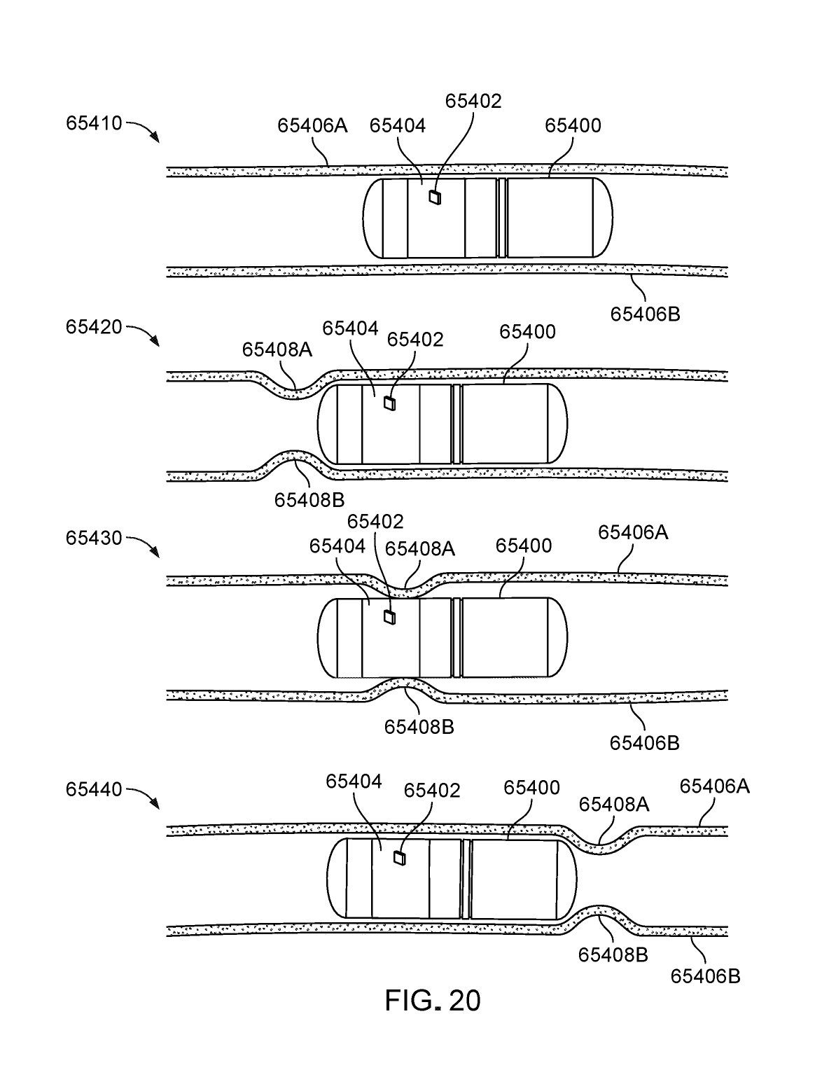

After entering duodenum 65310, ingestible device 65300 may be configured to detect a transition to the jejunum 65314 through the duodenojejunal flexure 65312. For example, ingestible device 65300 may be configured to use reflectances to detect peristaltic waves within the jejunum 65314, caused by the contraction of the smooth muscle tissue lining the walls of the jejunum 65314. In particular, ingestible device 65300 may be configured to begin periodically transmitting illumination (and measuring the resulting reflectances (e.g., via detector 65122 and illuminator 65124 of sensing sub-unit 65126 (FIG. 18)) at a sufficiently high frequency in order to detect muscle contractions within the jejunum 65314. Ingestible device 65300 may then determine that it has entered the jejunum 65314 in response to having detected either a first muscle contraction, or a predetermined number of muscle contractions (e.g., after having detected three muscle contractions in sequence). The interaction of ingestible device 65300 with the walls of jejunum 65314 is also discussed in relation to FIG. 20, and an example of this detection process is described in additional detail in relation to FIG. 25.

FIG. 20 is a diagram of an ingestible device during an example transit through a jejunum, in accordance with some embodiments of the disclosure. Diagrams 65410, 65420, 65430, and 65440 depict ingestible device 65400 as it traverses through a jejunum (e.g., jejunum 65314), and how ingestible device 65400 interacts with peristaltic waves formed by walls 65406A and 65406B (collectively, walls 65406) of the jejunum. In some implementations, ingestible device 65400 may include any portion of any other ingestible device discussed in this disclosure (e.g., ingestible device 65100 (FIG. 17) or ingestible device 65300 (FIG. 19)), and may be any suitable type of ingestible device with localization capabilities. For example, ingestible device 65400 may be substantially similar to the ingestible device 65300 (FIG. 67) or ingestible device 65100 (FIG. 18), with window 65404 being the same as window 65114 (FIG. 17), and sensing sub-unit 65402 being the same as sensing sub-unit 65126 (FIG. 18).

Diagram 65410 depicts ingestible device 400 within the jejunum, when the walls 65406 of the jejunum are relaxed. In some embodiments, the confined tube-like structure of the jejunum naturally causes ingestible device 65400 to be oriented longitudinally along the length of the jejunum, with window 65404 facing walls 65406. In this orientation, ingestible device 65400 may use sensing sub-unit 65402 to generate illumination (e.g., via illuminator 65124 (FIG. 18)) oriented towards walls 65406, and to detect the resulting reflectances (e.g., via detector 65122 (FIG. 18)) from the portion of the illumination reflected off of walls 65406 and back through window 65404. In some embodiments, ingestible device 65400 may be configured to use sensing sub-unit 65402 to generate illumination and measure the resulting reflectance with sufficient frequency to detect peristaltic waves within the jejunum. For instance, in a healthy human subject, peristaltic waves may occur at a rate of approximately 0.1 Hz to 0.2 Hz. Therefore, the ingestible device 65400 may be configured to generate illumination and measure the resulting reflectance at least once every 2.5 seconds (i.e., potentially minimum rate to detect a 0.2 Hz signal), and preferably at a higher rate, such as once every 0.5 seconds, which may improve the overall reliability of the detection process due to more data points being available. It is understood that the ingestible device 65400 need not gather measurements at precise intervals, and in some embodiments the ingestible device 65400 may be adapted to analyze data gathered at more irregular intervals, provided that there are still a sufficient number of appropriately spaced data points to detect 0.1 Hz to 0.2 Hz signals.

Diagram 65420 depicts ingestible device 65400 within the jejunum, when the walls 65406 of the jejunum begin to contract and form a peristaltic wave. Diagram 65420 depicts contracting portion 65408A of wall 65406A and contracting portion 65408B of wall 65406B (collectively, contracting portion 65408 of wall 65406) that form a peristaltic wave within the jejunum. The peristaltic wave proceeds along the length of the jejunum as different portions of wall 65406 contract and relax, causing it to appear as if contracting portions 65408 of wall 65406 proceed along the length of the jejunum (i.e., as depicted by contracting portions 65408 proceeding from left to right in diagrams 65410-65430). While in this position, ingestible device 65400 may detect a similar level of reflectance (e.g., through the use of illuminator 65124 and detector 65122 of sensing sub-unit 65126 (FIG. 18)) as detected when there is no peristaltic wave occurring (e.g., as detected when ingestible device 65400 is in the position indicated in diagram 65410).

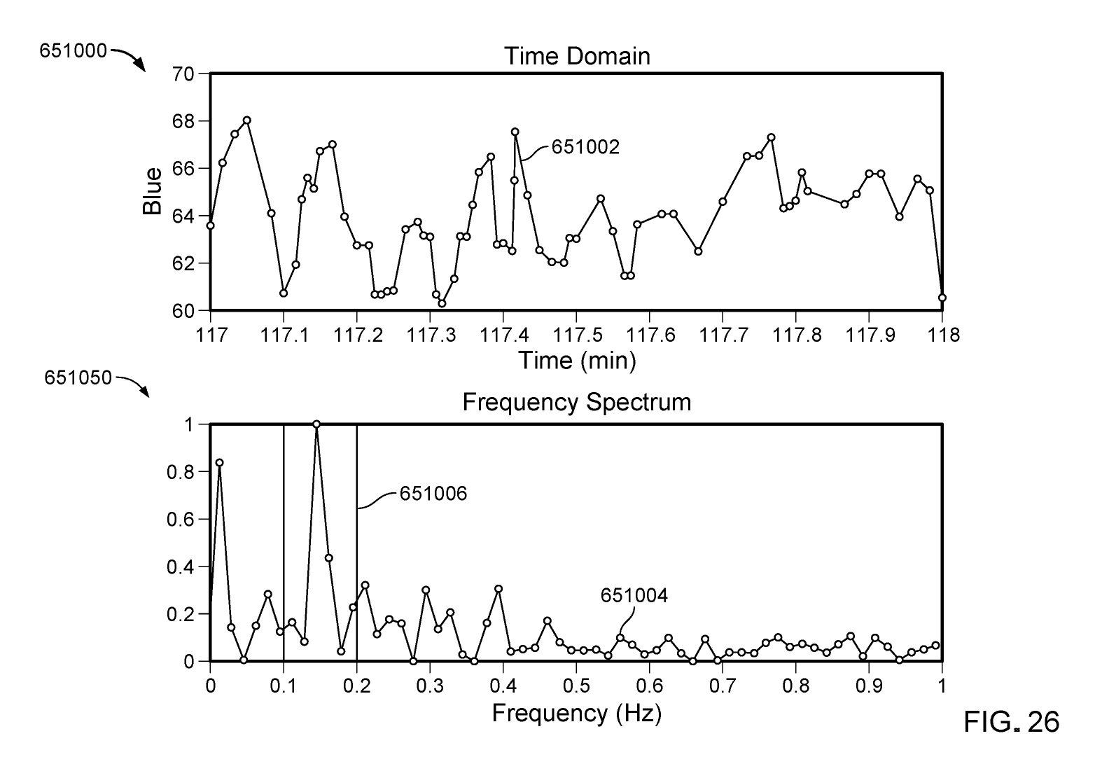

Diagram 65430 depicts ingestible device 65400 within the jejunum, when the walls 65406 of the jejunum continue to contract, squeezing around ingestible device 65400. As the peristaltic wave proceeds along the length of the jejunum, contracting portions 65408 of wall 65406 may squeeze tightly around ingestible device 65400, bringing the inner surface of wall 65406 into contact with window 65404. While in this position, ingestible device 65400 may detect a change in a reflectance detected as a result of illumination produced by sensing sub-unit 65402. The absolute value of the change in the measured reflectance may depend on several factors, such as the optical properties of the window 65404, the spectral components of the illumination, and the optical properties of the walls 65406. However, ingestible device 65400 may be configured to store a data set with the reflectance values over time, and search for periodic changes in the data set consistent with the frequency of the peristaltic waves (e.g., by analyzing the data set in the frequency domain, and searching for peaks between 0.1 Hz to 0.2 Hz). This may enable ingestible device 65400 to detect muscle contractions due to peristaltic waves without foreknowledge of the exact changes in reflectance signal amplitude that may occur as a result of detecting the muscle contractions of the peristaltic wave. An example procedure for detecting muscle contractions is discussed further in relation to FIG. 25, and an example of a reflectance data set gathered while ingestible device 65400 is located within the jejunum is discussed in relation to FIG. 26.

Diagram 440 depicts ingestible device 65400 within the jejunum, when the peristaltic wave has moved past ingestible device 65400. Diagram 65440 depicts contracting portions 65408 that form the peristaltic wave within the jejunum having moved past the end of ingestible device 65400. The peristaltic wave proceeds along the length of the jejunum as different portions of wall 65406 contract and relax, causing it to appear as if contracting portions 65408 of wall 65406 proceed along the length of the jejunum (i.e., as depicted by contracting portions 65408 proceeding from left to right in diagrams 65410-65430). While in this position, ingestible device 65400 may detect a similar level of reflectance (e.g., through the use of illuminator 65124 and detector 65122 of sensing sub-unit 65126 (FIG. 18)) as detected when there is no peristaltic wave occurring (e.g., as detected when ingestible device 65400 is in the position indicated in diagram 65410, or diagram 65420).

Depending on the species of the subject, peristaltic waves may occur with relatively predictable regularity. After the peristaltic wave has passed over ingestible device 65400 (e.g., as depicted in diagram 65440), the walls 65406 of the jejunum may relax again (e.g., as depicted in diagram 65410), until the next peristaltic wave begins to form. In some embodiments, ingestible device 65400 may be configured to continue to gather reflectance value data while it is within the GI tract, and may store a data set with the reflectance values over time. This may allow ingestible device 65400 to detect each of the muscle contractions as the peristaltic wave passes over ingestible device 65400 (e.g., as depicted in diagram 65430), and may enable ingestible device 65400 to both count the number of muscle contractions that occur, and to determine that a current location of the ingestible device 65400 is within the jejunum. For example, ingestible device 65400 may be configured to monitor for possible muscle contractions while is inside either the stomach or the duodenum, and may determine that ingestible device 65400 has moved to the jejunum in response to detecting a muscle contraction consistent with a peristaltic wave.

FIG. 21 is a flowchart illustrating some aspects of a localization process used by the ingestible device. Although FIG. 21 may be described in connection with the ingestible device 65100 for illustrative purposes, this is not intended to be limiting, and either portions or the entirety of the localization procedure 65500 described in FIG. 21 may be applied to any device discussed in this application (e.g., the ingestible devices 65100, 65300, and 65400), and any of the ingestible devices may be used to perform one or more parts of the process described in FIG. 21. Furthermore, the features of FIG. 21 may be combined with any other systems, methods or processes described in this application. For example, portions of the process in FIG. 21 may be integrated into or combined with the pyloric transition detection procedure described by FIG. 22, or the jejunum detection process described by FIG. 25.

At 65502, the ingestible device (e.g., ingestible device 65100, 65300, or 65400) gathers measurements (e.g., through detector 65122 (FIG. 17)) of ambient light. For example, ingestible device 65100 may be configured to periodically measure (e.g., through detector 65122 (FIG. 17)) the level of ambient light in the environment surrounding ingestible device 65100. In some embodiments, the type of ambient light being measured may depend on the configuration of detector 65122 within ingestible device 65100. For example, if detector 65122 is configured to measure red, green, and blue wavelengths of light, ingestible device 65100 may be configured to measure the ambient amount of red, green, and blue light from the surrounding environment. In some embodiments, the amount of ambient light measured by ingestible device 65100 will be larger in the area external to the body (e.g., a well-lit room where ingestible device 65100 is being administered to a subject) and in the oral cavity of the subject, as compared to the ambient level of light measured by ingestible device 65100 when inside of an esophagus, stomach, or other portion of the GI tract (e.g., esophagus 65302, stomach 65306, duodenum 65310, or jejunum 65314 (FIG. 19)).

At 65504, the ingestible device (e.g., ingestible device 65100, 65300, or 65400) determines (e.g., via control circuitry within PCB 65120 (FIG. 18)) whether the ingestible device has detected entry into the GI tract. For example, ingestible device 65100 may be configured to determine when the most recent measurement of ambient light (e.g., the measurement gathered at 65502) indicates that the ingestible device has entered the GI tract. For instance, the first time that ingestible device 65100 gatherers a measurement of ambient light at 65502, ingestible device 65100 may store that measurement (e.g., via storage circuitry within PCB 65120 (FIG. 18)) as a typical level of ambient light external to the body. Ingestible device 65100 may be configured to then compare the most recent measurement of ambient light to the typical level of ambient light external to the body (e.g., via control circuitry within PCB 65120 (FIG. 18)), and determine that ingestible device 65100 has entered the GI tract when the most recent measurement of ambient light is substantially smaller than the typical level of ambient light external to the body. For example, ingestible device 65100 may be configured to detect that it has entered the GI tract in response to determining that the most recent measurement of ambient light is less than or equal to 20% of the typical level of ambient light external to the body. If ingestible device 65100 determines that it has detected entry into the GI tract (e.g., that ingestible device 65100 has entered at least the esophagus 65302 (FIG. 19)), process 65500 proceeds to 65506. Alternately, if ingestible device 65100 determines that it has not detected entry into the GI tract (e.g., as a result of the most recent measurement being similar to the typical level of ambient light external to the body), process 65500 proceeds back to 65502 where the ingestible device 65100 gathers further measurements. For instance, ingestible device 65100 may be configured to wait a predetermined amount of time (e.g., five seconds, ten seconds, etc.), and then gather another measurement of the level of ambient light from the environment surrounding ingestible device 65100.

At 65506, the ingestible device (e.g., ingestible device 65100, 65300, or 65400) waits for a transition from the esophagus to the stomach (e.g., from esophagus 65302 to stomach 65306 (FIG. 19)). For example, ingestible device 65100 may be configured to determine that it has entered the stomach (e.g., stomach 65306 (FIG. 19)) after waiting a predetermined period of time after having entered the GI tract. For instance, a typical esophageal transit time in a human patient may be on the order of 15-30 seconds. In this case, after having detected that ingestible device 65100 has entered the GI tract at 65504 (i.e., after detecting that ingestible device 65100 has reached at least esophagus 65302 (FIG. 19)), ingestible device 65100 may be configured to wait one minute, or a similar amount of time longer than the typical esophageal transmit time (e.g., ninety-seconds), before automatically determining that ingestible device 65100 has entered at least the stomach (e.g., stomach 65306 (FIG. 19)).

In some embodiments, the ingestible device (e.g., ingestible device 65100, 65300, or 65400) may also determine it has entered the stomach based on measurements of pH or temperature. For example, ingestible device 65100 may be configured to determine that it has entered the stomach if a temperature of ingestible device has increased to at least 31 degrees Celsius (i.e., consistent with the temperature inside the stomach), or if a measured pH of the environment surrounding ingestible device 65100 is sufficiently acidic (i.e., consistent with the acidic nature of gastric juices that may be found inside the stomach).

At 65508, the ingestible device (e.g., ingestible device 65100, 65300, or 65400) stores data indicating the ingestible device has entered the stomach (e.g., stomach 306 (FIG. 19)). For example, after having waited a sufficient amount of time at 65506, ingestible device 65100 may store data (e.g., within storage circuitry of PCB 65120 (FIG. 18)) indicative of ingestible device 65100 having entered at least the stomach. Once ingestible device 65100 reaches at least the stomach, process 65500 proceeds to 65510 where ingestible device 65100 may be configured to gather data to detect entry into the duodenum (e.g., duodenum 65310 (FIG. 19)).

In some embodiments, process 65500 may also simultaneously proceed from 65508 to 65520, where ingestible device 65100 may be configured to gather data in order to detect muscle contractions and detect entry into the jejunum (e.g., jejunum 65314 (FIG. 19)). In some embodiments, ingestible device 65100 may be configured to simultaneously monitor for entry into the duodenum at 65516-65518, as well as detect for entry into the jejunum at 65520-65524. This may allow ingestible device 65100 to determine when it has entered the jejunum (e.g., as a result of detecting muscle contractions), even when it fails to first detect entry into the duodenum (e.g., as a result of very quick transit times of the ingestible device through the duodenum).

At 65510, the ingestible device (e.g., ingestible device 65100,65300, or 65400) gathers measurements of green and blue reflectance levels (e.g., through the use of illuminator 65124 and detector 65122 of sensing sub-unit 65126 (FIG. 18)) while in the stomach (e.g., stomach 65306 (FIG. 19)). For example, ingestible device 100 may be configured to periodically gather measurements of green and blue reflectance levels while in the stomach. For instance, ingestible device 65100 may be configured to transmit a green illumination and a blue illumination (e.g., via illuminator 65124 (FIG. 18)) every five to fifteen seconds, and measure the resulting reflectance (e.g., via detector 65122 (FIG. 18)). Every time that ingestible device 65100 gathers a new set of measurements, the measurements may be added to a stored data set (e.g., stored within memory circuitry of PCB 65120 (FIG. 18)). The ingestible device 65100 may then use this data set to determine whether or not ingestible device 65100 is still within a stomach (e.g., stomach 65306 (FIG. 19)), or a duodenum (e.g., duodenum 65310 (FIG. 19)).

In some embodiments, the ingestible device (e.g., ingestible device 65100, 65300, or 65400) may be configured to detect a first reflectance based on generating an illumination of a first wavelength in approximately the green spectrum of light (between 495-600 nm), and detecting a second reflectance based on generating an illumination of the second wavelength in approximately the blue spectrum of light (between 400-495 nm). In some embodiments, the ingestible device may ensure that the illumination in the green spectrum and the illumination in the blue spectrum have wavelengths separated by at least 50 nm. This may enable ingestible device 65100 to sufficiently distinguish between the two wavelengths when detecting the reflectances (e.g., via detector 65122 (FIG. 18)). It is understood that the separation of 50 nm is intended to be illustrative, and not limiting, and depending on the accuracy of the detectors within ingestible device 65100, smaller separations may be possible to be used.

At 65512, the ingestible device (e.g., ingestible device 65100, 65300, or 65400) determines (e.g., using control circuitry within PCB 65120 (FIG. 18)) whether the ingestible device has detected a transition from the stomach (e.g., stomach 65306 (FIG. 19)) to a duodenum (e.g., duodenum 65310 (FIG. 19)) based on a ratio of green and blue (G/B) reflectance levels. For example, ingestible device 65100 may obtain (e.g., from memory circuitry of PCB 65120 (FIG. 18)) a data set containing historical data for the respective ratio of the green reflectance to the blue reflectance as measured at a respective time. Generally speaking, a duodenum (e.g., duodenum 65310 (FIG. 19)) of a human subject reflects a higher ratio of green light to blue light, as compared to the ratio of green light to blue light that is reflected by a stomach (e.g., stomach 65306 (FIG. 19)). Based on this, ingestible device 65100 may be configured to take a first set of ratios from the data set, representing the result of recent measurements, and compare them to a second set of ratios from the data set, representing the results of past measurements. When the ingestible device 65100 determines that the mean value of the first set of ratios is substantially larger than the mean value of the second set of ratios (i.e., that the ratio of reflected green light to reflected blue light has increased), the ingestible device 65100 may determine that it has entered the duodenum (e.g., duodenum 65310 (FIG. 19)) from the stomach (e.g., stomach 65306 (FIG. 18)). If the ingestible device 65100 detects a transition from the stomach (e.g., stomach 65306 (FIG. 19)) to a duodenum (e.g., duodenum 65310 (FIG. 19)), process 65500 proceeds to 65514, where ingestible device 65100 stores data indicating that the ingestible device 65100 has entered the duodenum (e.g., duodenum 65310 (FIG. 19)). Alternatively, if the ingestible device determines that the ingestible device has not transitioned from the stomach (e.g., stomach 65306 (FIG. 19)) to the duodenum (e.g., duodenum 65310 (FIG. 19)), process 65500 proceeds back to 65510 to gather more measurements of green and blue reflectance levels while still in the stomach (e.g., stomach 65306 (FIG. 19)). An example procedure for using measurements of green and blue reflectances to monitor for transitions between the stomach and the duodenum is discussed in greater detail in relation to FIG. 22.

In some embodiments, the first time that ingestible device 65100 detects a transition from the stomach (e.g., stomach 65306 (FIG. 19)) to the duodenum (e.g., duodenum 65310 (FIG. 19)), ingestible device 65100 may be configured to take a mean of the second set of data, (e.g., the set of data previously recorded while in stomach 65306 (FIG. 19)) and store this as a typical ratio of green light to blue light detected within the stomach (e.g., stomach 65306 (FIG. 19)) (e.g., within memory circuitry of PCB 65120 (FIG. 19)). This stored information may later be used by ingestible device 65100 to determine when ingestible device 65100 re-enters the stomach (e.g., stomach 65306 (FIG. 19)) from the duodenum (e.g., duodenum 65310 (FIG. 19)) as a result of a reverse pyloric transition.

At 65514, the ingestible device (e.g., ingestible device 65100, 65300, or 65400) stores data indicating that the ingestible device has entered the duodenum (e.g., duodenum 65310 (FIG. 19)). For example, ingestible device 65100 may store a flag within local memory (e.g., memory circuitry of PCB 65120) indicating that the ingestible device 65100 is currently in the duodenum. In some embodiments, the ingestible device 65100 may also store a timestamp indicating the time when ingestible device 65100 entered the duodenum. Once ingestible device 65100 reaches the duodenum, process 65500 proceeds to 65520 where ingestible device 65100 may be configured to gather data in order to detect muscle contractions and detect entry into the jejunum (e.g., jejunum 65314 (FIG. 19)). Process 65500 also proceeds from 65514 to 65516, where ingestible device 65100 may be configured to gather data additional data in order to detect re-entry into the stomach (e.g., stomach 65306 (FIG. 19)) from the duodenum (e.g., duodenum 65310 (FIG. 19)).

At 65516, the ingestible device (e.g., ingestible device 65100, 65300, or 65400) gathers measurements (e.g., via sensing sub-unit 65126 (FIG. 18)) of green and blue reflectance levels while in the duodenum (e.g., duodenum 65310 (FIG. 19)). For example, ingestible device 65100 may be configured to periodically gather measurements (e.g., via sensing sub-unit 65126 (FIG. 18)) of green and blue reflectance levels while in the duodenum, similar to the measurements made at 65510 while in the stomach. For instance, ingestible device 65100 may be configured to transmit a green illumination and a blue illumination (e.g., via illuminator 65124 (FIG. 18)) every five to fifteen seconds, and measure the resulting reflectance (e.g., via detector 65122 (FIG. 18)). Every time that ingestible device 65100 gathers a new set of measurements, the measurements may be added to a stored data set (e.g., stored within memory circuitry of PCB 65120 (FIG. 18)). The ingestible device 65100 may then use this data set to determine whether or not ingestible device 65100 is still within the duodenum (e.g., duodenum 65310 (FIG. 19)), or if the ingestible device 65100 has transitioned back into the stomach (e.g., stomach 65306 (FIG. 19)).

At 65518, the ingestible device (e.g., ingestible device 65100, 65300, or 65400) determines a transition from the duodenum (e.g., duodenum 65310 (FIG. 19)) to the stomach (e.g., stomach 65306 (FIG. 19)) based on a ratio of the measured green reflectance levels to the measured blue reflectance levels. In some embodiments, ingestible device 65100 may compare the ratio of the measured green reflectance levels to the measured blue reflectance levels recently gathered by ingestible device 65100 (e.g., measurements gathered at 65516), and determine whether or not the ratio of the measured green reflectance levels to the measured blue reflectance levels is similar to the average ratio of the measured green reflectance levels to the measured blue reflectance levels seen in the stomach (e.g., stomach 65306 (FIG. 19)). For instance, ingestible device 65100 may retrieve data (e.g., from memory circuitry of PCB 65120 (FIG. 18)) indicative of the average ratio of the measured green reflectance levels to the measured blue reflectance levels seen in the stomach, and determine that ingestible device 65100 has transitioned back to the stomach if the recently measured ratio of the measured green reflectance levels to the measured blue reflectance levels is sufficiently similar to the average level in the stomach (e.g., within 20% of the average ratio of the measured green reflectance levels to the measured blue reflectance levels seen in the stomach, or within any other suitable threshold level). If the ingestible device detects a transition from the duodenum (e.g., duodenum 65310 (FIG. 19)) to the stomach (e.g., stomach 65306 (FIG. 19)), process 65500 proceeds to 65508 to store data indicating the ingestible device has entered the stomach (e.g., stomach 65306 (FIG. 19)), and continues to monitor for further transitions. Alternatively, if the ingestible device does not detect a transition from the duodenum (e.g., duodenum 65310 (FIG. 19)) to the stomach (e.g., stomach 65306 (FIG. 19)), process 65500 proceeds to 65516 to gather additional measurements of green and blue reflectance levels while in the duodenum (e.g., duodenum 65310 (FIG. 19)), which may be used to continuously monitor for possible transitions back into the stomach. An example procedure for using measurements of green and blue reflectances to monitor for transitions between the stomach and the duodenum is discussed in greater detail in relation to FIG. 22.

At 65520, the ingestible device (e.g., ingestible device 65100, 65300, or 65400) gathers periodic measurements of the reflectance levels (e.g., via sensing sub-unit 65126 (FIG. 18)) while in the duodenum (e.g., duodenum 65310 (FIG. 19)). In some embodiments, the ingestible device (e.g., ingestible device 65100, 65300, or 65400) may gather similar periodic measurements while in the stomach as well. In some embodiments, these periodic measurements may enable ingestible device 65100 to detect muscle contractions (e.g., muscle contractions due to a peristaltic wave as discussed in relation to FIG. 20), which may be indicative of entry into a jejunum (e.g., jejunum 65314 (FIG. 19)). Ingestible device 65100 may be configured to gather periodic measurements using any suitable wavelength of illumination (e.g., by generating illumination using illuminator 65124, and detecting the resulting reflectance using detector 65122 (FIG. 18)), or combinations of wavelengths of illumination. For example, in some embodiments, ingestible device 65100 may be configured to generate red, green, and blue illumination, store separate data sets indicative of red, green, and blue illumination, and analyze each of the data sets separately to search for frequency components in the recorded data indicative of detected muscle contractions. In some embodiments, the measurements gathered by ingestible device 65100 at 65520 may be sufficiently fast as to detect peristaltic waves in a subject. For instance, in a healthy human subject, peristaltic waves may occur at a rate of approximately 0.1 Hz to 0.2 Hz. Therefore, the ingestible device 65400 may be configured to generate illumination and measure the resulting reflectance at least once every 2.5 seconds (i.e., potentially minimum rate to detect a 0.2 Hz signal), and preferably at a higher rate, such as once every 0.5 seconds or faster, and store values indicative of the resulting reflectances in a data set (e.g., within memory circuitry of PCB 65120 (FIG. 18)). After gathering additional data (e.g., after gathering one new data point, or a predetermined number of new data points), process 65500 proceeds to 65522, where ingestible device 65100 determines whether or not a muscle contraction has been detected.

At 65522, the ingestible device (e.g., ingestible device 65100, 65300, or 65400) determines (e.g., via control circuitry within PCB 65120 (FIG. 18)) whether the ingestible device detects a muscle contraction based on the measurements of reflectance levels (e.g., as gathered by sensing sub-unit 65126 (FIG. 18)). For example, ingestible device 65100 may obtain a fixed amount of data stored as a result of measurements made at 65520 (e.g., retrieve the past minute of data from memory circuitry within PCB 65120 (FIG. 18)). Ingestible device 65100 may then convert the obtained data into the frequency domain, and search for peaks in a frequency range that would be consistent with peristaltic waves. For example, in a healthy human subject, peristaltic waves may occur at a rate of approximately 0.1 Hz to 0.2 Hz, and an ingestible device 65100 may be configured to search for peaks in the frequency domain representation of the data between 0.1 Hz and 0.2 Hz above a threshold value. If the ingestible device 65100 detects a contraction based on the reflectance levels (e.g., based on detecting peaks in the frequency domain representation of the data between 0.1 Hz and 0.2 Hz), process 65500 proceeds to 65524 to store data indicating that the device has entered the jejunum. Alternatively, if the ingestible device 65100 does not detect a muscle contraction, process 65500 proceeds to 65520 to gather periodic measurements of the reflectance levels while in the duodenum (e.g., duodenum 65310 (FIG. 19)). In some embodiments, the ingestible device (e.g., ingestible device 65100, 65300, or 65400) may store data (e.g., within memory circuitry of PCB 65120 (FIG. 18)) indicating that a muscle contraction was detected, and process 65500 will not proceed from 65522 to 65524 until a sufficient number of muscle contractions have been detected.

At 65524, the ingestible device (e.g., ingestible device 65100, 65300, or 65400) stores data (e.g., within memory circuitry of PCB 65120 (FIG. 18)) indicating that the device has entered the jejunum (e.g., jejunum 65314 (FIG. 19)). For example, in response to detecting that muscle contraction has occurred at 65522, ingestible device 65100 may determine that it has entered the jejunum 65314, and is no longer inside of the duodenum (e.g., duodenum 65310 (FIG. 19)) or the stomach (e.g., stomach 65306 (FIG. 19)). In some embodiments, the ingestible device 65100 may continue to measure muscle contractions while in the jejunum, and may store data indicative of the frequency, number, or strength of the muscle contractions over time (e.g., within memory circuitry of PCB 65120 (FIG. 18)). In some embodiments, the ingestible device 65100 may also be configured to monitor for one or more transitions. Such transitions can include a transition from the jejunum to the ileum, an ileoceacal transition from the ileum to the cecum, a transition from the cecum to the colon, or detect exit from the body (e.g., by measuring reflectances, temperature, or levels of ambient light).

In some embodiments, the ingestible device (e.g., ingestible device 65100, 65300, or 65400) may also determine that it has entered the jejunum (e.g., jejunum 65314 (FIG. 19)) after a pre-determined amount of time has passed after having detected entry into the duodenum (e.g., duodenum 65310 (FIG. 19)). For example, barring a reverse pyloric transition from the duodenum (e.g., duodenum 65310 (FIG. 19)) back to the stomach (e.g., stomach 65306 (FIG. 19)), the typical transit time for an ingestible device to reach the jejunum from the duodenum in a healthy human subject is less than three minutes. In some embodiments, the ingestible device (e.g., ingestible device 65100, 65300, or 65400) may therefore be configured to automatically determine that it has entered the jejunum after spending at least three minutes within the duodenum. This determination may be made separately from the determination made based on measured muscle contractions (e.g., the determination made at 65522), and in some embodiments, ingestible device 65100 may determine that it has entered the jejunum in response to either detecting muscle contractions, or after three minutes has elapsed from having entered the duodenum (e.g., as determined by storing data at 65514 indicative of the time that ingestible device entered the duodenum).

For illustrative purposes, 65512-65518 of process 65500 describe the ingestible device (e.g., ingestible device 65100, 65300, or 65400) measuring green reflectances and blue reflectances, calculating a ratio of the two reflectances, and using this information to determine when the ingestible device has transitioned between the duodenum and stomach. However, in some embodiments, other wavelengths of light may be used other than green and blue, provided that the wavelengths of light chosen have different reflective properties within the stomach and the duodenum (e.g., as a result of different reflection coefficients of the stomach tissue and the tissue of the duodenum).

It will be understood that the steps and descriptions of the flowcharts of this disclosure, including FIG. 21, are merely illustrative. Any of the steps and descriptions of the flowcharts, including FIG. 21, may be modified, omitted, rearranged, and performed in alternate orders or in parallel, two or more of the steps may be combined, or any additional steps may be added, without departing from the scope of the present disclosure. For example, the ingestible device 65100 may calculate the mean and the standard deviation of multiple data sets in parallel in order to speed up the overall computation time. As another example, ingestible device 65100 may gather data periodic measurements and detect possible muscle contractions (e.g., at 65520-65522) while simultaneously gathering green and blue reflectance levels to determine transitions to and from the stomach and duodenum (e.g., at 65510-65518). Furthermore, it should be noted that the steps and descriptions of FIG. 21 may be combined with any other system, device, or method described in this application, including processes 65600 (FIGS. 22) and 65900 (FIG. 25), and any of the ingestible devices or systems discussed in this application (e.g., ingestible devices 65100, 65300, or 65400) could be used to perform one or more of the steps in FIG. 21.

FIG. 22 is a flowchart illustrating some aspects of a process for detecting transitions from a stomach to a duodenum and from a duodenum back to a stomach, which may be used when determining a location of an ingestible device as it transits through a gastrointestinal (GI) tract, in accordance with some embodiments of the disclosure. In some embodiments, process 65600 may begin when an ingestible device first detects that it has entered the stomach, and will continue as long as the ingestible device determines that it is within the stomach or the duodenum. In some embodiments, process 65600 may only be terminated when an ingestible device determines that it has entered the jejunum, or otherwise progressed past the duodenum and the stomach. Although FIG. 22 may be described in connection with the ingestible device 65100 for illustrative purposes, this is not intended to be limiting, and either portions or the entirety of the duodenum detection process 65600 described in FIG. 22 may be applied to any device discussed in this application (e.g., the ingestible devices 65100, 65300, or 65400), and any of the ingestible devices may be used to perform one or more parts of the process described in FIG. 22. Furthermore, the features of FIG. 22 may be combined with any other systems, methods or processes described in this application. For example, portions of the process described by the process in FIG. 22 may be integrated into process 65500 discussed in relation to FIG. 21.

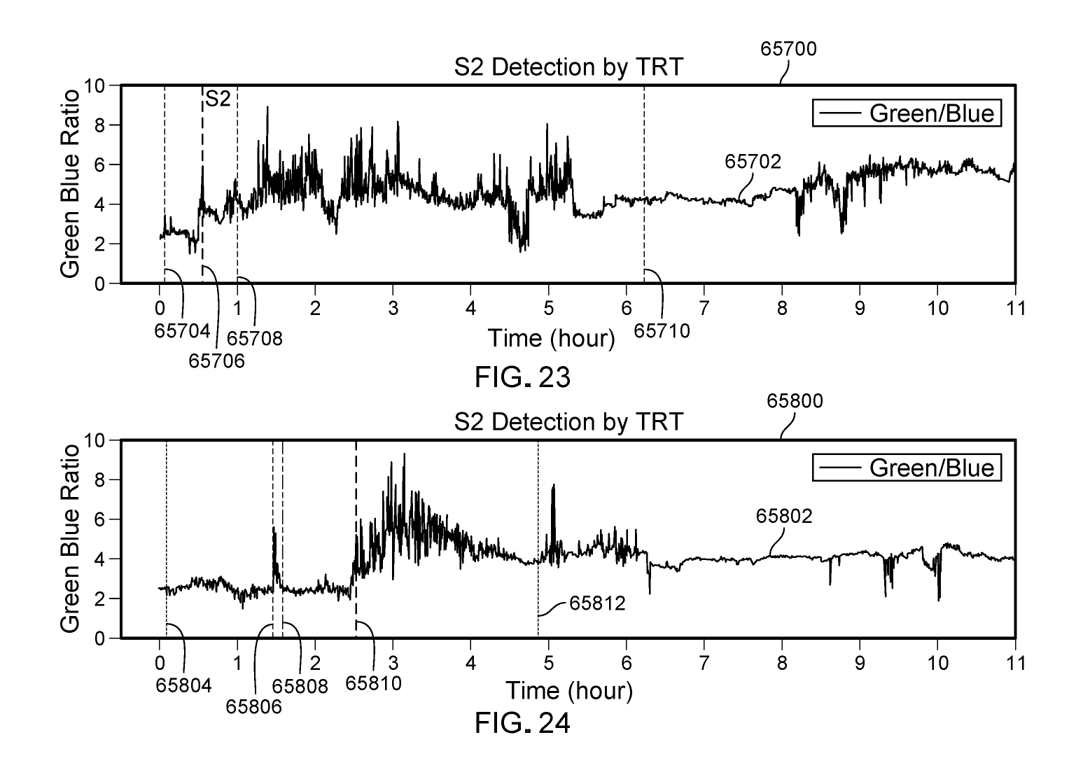

At 65602, the ingestible device (e.g., ingestible device 65100, 65300, or 65400) retrieves a data set (e.g., from memory circuitry within PCB 65120 (FIG. 18)) with ratios of the measured green reflectance levels to the measured blue reflectance levels over time. For example, ingestible device 65100 may retrieve a data set from PCB 65120 containing recently recorded ratios of the measured green reflectance levels to the measured blue reflectance levels (e.g., as recorded at 65510 or 65516 of process 65500 (FIG. 21)). In some embodiments, the retrieved data set may include the ratios of the measured green reflectance levels to the measured blue reflectance levels over time. Example plots of data sets of ratios of the measured green reflectance levels to the measured blue reflectance levels are discussed further in relation to FIG. 23 and FIG. 24.

At 65604, the ingestible device (e.g., ingestible device 65100, 65300, or 65400) includes a new measurement (e.g., as made with sensing sub-unit 65126 (FIG. 18)) of a ratio of the measured green reflectance level to the measured blue reflectance level in the data set. For example, ingestible device 65100 may be configured to occasionally record new data by transmitting green and blue illumination (e.g., via illuminator 65124 (FIG. 18)), detecting the amount of reflectance received due to the green and blue illumination (e.g., via detector 65122 (FIG. 18)), and storing data indicative of the amount of the received reflectance (e.g., in memory circuitry of PCB 65120 (FIG. 18)). The ingestible device 65100 may be configured to record new data every five to fifteen seconds, or at any other convenient interval of time. For illustrative purposes, ingestible device 65100 is described as storing and retrieving the ratio of the measured green reflectance levels to the measured blue reflectance levels (e.g., if the amount of detected green reflectance was identical to the amount of detected blue reflectance at a given time, the ratio of the green and blue reflectances would be “1.0” at that given time); however, it is understood that the green reflectance data and the blue reflectance data may be stored separately within the memory of ingestible device 65100 (e.g., stored as two separate data sets within memory circuitry of PCB 65120 (FIG. 18)).

At 65606, the ingestible device (e.g., ingestible device 65100, 65300, or 65400) retrieves a first subset of recent data by applying a first sliding window filter to the data set. For example, ingestible device 65100 may use a sliding window filter to obtain a predetermined amount of the most recent data within the data set, which may include any new values of the ratio of the measured green reflectance level to the measured blue reflectance level obtained at 65604. For instance, the ingestible device may be configured to select between ten and forty data points from the data set, or ingestible device 65100 may be configured to select a predetermined range of data values between fifteen seconds of data and five minutes of data. In some embodiments, other ranges of data may be selected, depending on how frequently measurements are recorded, and the particular application at hand. For instance, any suitable amount of data may be selected in the sliding window, provided that it is sufficient to detect statistically significant differences between the data selected in a second sliding window (e.g., the second subset of data selected at 65614).

In some embodiments, the ingestible device (e.g., ingestible device 65100, 65300, or 65400) may also be configured to remove outliers from the data set, or to smooth out unwanted noise in the data set. For example, ingestible device 65100 may select the first subset of data, or any other subset of data, by first obtaining a raw set of values by applying a window filter to the data set (e.g., selecting a particular range of data to be included). Ingestible device 65100 may then be configured to identify outliers in the raw set of values; for instance, by identifying data points that are over three standard deviations away from the mean value of the raw set of values, or any other suitable threshold. Ingestible device 65100 may then determine the subset of data by removing outliers from the raw set of values. This may enable ingestible device 65100 to avoid spurious information when determining whether or not it is located within the stomach or the duodenum.

At 65608, the ingestible device (e.g., ingestible device 65100, 65300, or 65400) determines whether the most recently detected location was the duodenum (e.g., duodenum 65310 (FIG. 19)). In some embodiments, ingestible device 65100 may store a data flag (e.g., within memory circuitry of PCB 65120 (FIG. 18)) indicating the most recent portion of the GI tract that the ingestible device 65100 detected itself to be within. For instance, every time ingestible device 65100 detects entry to the stomach (e.g., detects entry into stomach 65306 (FIG. 19) as a result of the decision made at 65610), a flag is stored in memory indicating the ingestible device 65100 is in the stomach (e.g., as part of storing data at 65612). If ingestible device 65100 subsequently detects entry into the duodenum (e.g., detects entry into duodenum 65310 (FIG. 19) as a result of a decision made at 65624), another different flag is stored in memory indicating that the ingestible device 65100 is in the duodenum (e.g., as part of storing data at 65624). In this case, ingestible device 65100 may retrieve the most recently stored flag at 65608, and determine whether or not the flag indicates that the ingestible device 65100 was most recently within the duodenum. If ingestible device 65100 detects that it was most recently in the duodenum, process 65600 proceeds to 65610 where the ingestible device compares the recent measurements of the ratios of the measured green reflectance levels to the measured blue reflectance levels (e.g., measurements that include the recent measurement made at 65606) to the typical ratios measured within the stomach, and uses this information to determine whether a reverse pyloric transition from the duodenum back to the stomach has occurred. Alternately, if ingestible device 65100 detects that it was not most recently in the duodenum (e.g., because it was in the stomach instead), process 65600 proceeds to 65614 where the ingestible device compares the recent measurements of the ratios of the measured green reflectance levels to the measured blue reflectance levels (e.g., measurements that include the recent measurement made at 65606) to past measurements, and uses this information to determine whether a pyloric transition from the stomach to the duodenum has occurred.

Process 65600 proceeds from 65608 to 65610 when the ingestible device determined that it was most recently in the duodenum. At 65610, the ingestible device (e.g., ingestible device 65100, 65300, or 65400) determines (e.g., via control circuitry within PCB 65120 (FIG. 18)) whether the current G/B signal is similar to a recorded average G/B signal in the stomach. For example, ingestible device 65100 may be configured to have previously stored data (e.g., within memory circuitry of PCB 65120 (FIG. 18)) indicative of the average ratio of the measured green reflectance levels to the measured blue reflectance levels measured in the stomach. Ingestible device 65100 may then retrieve this stored data indicative of the average ratio of the measured green reflectance levels to the measured blue reflectance levels in the stomach, and compare this against the recent measurements in order to determine whether or not ingestible device 65100 has returned back to the stomach from the duodenum. For instance, ingestible device 65100 may determine if the mean value of the first subset of recent data (i.e., the average value of the recently measured ratios of the measured green reflectance levels to the measured blue reflectance levels) is less than the average ratio of the measured green reflectance levels to the measured blue reflectance levels within the stomach, or less that the average ratio measured within the stomach plus a predetermined number times the standard deviation of the ratios measured within the stomach. For instance, if the average ratio of the measured green reflectance levels to the measured blue reflectance levels in the stomach was “1,” with a standard deviation of “0.2,” ingestible device 100 may determine whether or not the mean value of the first subset of data is less than “1.0+k*0.2,” where “k” is a number between zero and five. It is understood that, in some embodiments, the ingestible device 65100 may be configured to use a different threshold level to determine whether or not the mean value of the first subset of recent data is sufficiently similar to the average ratio of the measured green reflectance levels to the measured blue reflectance levels within the stomach. In response to determining that the recent ratio of the measured green reflectance levels to the measured blue reflectance levels is similar to the average ratio of measured green and blue reflectance levels seen in the stomach, process 65600 proceeds to 65612 where ingestible device 65100 stores data indicating that it has re-entered the stomach from the duodenum. Alternately, in response to determining that the recent ratio of measured green and blue reflectance levels is sufficiently different from the average ratio of measured green and blue reflectance levels seen in the stomach, ingestible device 65100 proceeds directly to 65604, and continues to obtain new data on an ongoing basis.

At 65612, the ingestible device (e.g., ingestible device 65100, 65300, or 65400) stores data indicating a reverse pyloric transition from the duodenum to the stomach was detected. For example, ingestible device 65100 may store a data flag (e.g., within memory circuitry of PCB 65120 (FIG. 18)) indicating that the ingestible device 65100 most recently detected itself to be within the stomach portion of the GI tract (e.g., stomach 65306 (FIG. 19)). In some embodiments, ingestible device 65100 may also store data (e.g., within memory circuitry of PCB 65120 (FIG. 18)) indicating a time that ingestible device 65100 detected the reverse pyloric transition from the duodenum to the stomach. This information may be used by ingestible device 65100 at 65608, and as a result process 65600 may proceed from 65608 to 65614, rather than proceeding from 65618 to 65610. After ingestible device 65100 stores the data indicating a reverse pyloric transition from the duodenum to the stomach was detected, process 65600 proceeds to 65604 where ingestible device 65100 continues to gather additional measurements, and continues to monitor for further transitions between the stomach and the duodenum.

Process 65600 proceeds from 65608 to 65614 when the ingestible device determined that it was not most recently in the duodenum (e.g., as a result of having most recently been in the stomach instead). At 65614, the ingestible device (e.g., ingestible device 65100, 65300, or 65400) retrieves a second subset of previous data by applying a second sliding window filter to the data set. For example, ingestible device 65100 may use a sliding window filter to obtain a predetermined amount of older data from a past time range, which may be separated from recent time range used to select the first subset of data gathered at 65606 by a predetermined period of time. In some embodiments, any suitable amount of data may be selected by the first and second window filters, and the first and second window filters may be separated by any appropriate predetermined amount of time. For example, in some embodiments, the first window filter and the second window filter may each be configured to select a predetermined range of data values from the data set, the predetermined range being between fifteen seconds of data and five minutes of data. In some embodiments, the recent measurements and the past measurements may then be separated by a predetermined period of time that is between one to five times the predetermined range of data values. For instance, ingestible device 65100 may select the first subset of data and the second subset of data to each be one minute of data selected from the dataset (i.e., selected to have a predetermined range of one minute), and the first subset of data and the second subset of data are selected from recorded measurements that are at least two minutes apart (i.e., the predetermined period of time is two minutes, which is twice the range used to select the subsets of data using the window filters). As another example, ingestible device 100 may select the first subset of data and the second subset of data to each be five minutes of data selected from the dataset (i.e., selected to have a predetermined range of five minutes), and the first subset of data and the second subset of data are selected from recorded measurements that are at least 10 minutes apart (i.e., the predetermined period of time is two minutes, which is twice the range used to select the subsets of data using the window filters).

In some embodiments, if ingestible device 65100 recently transitioned to the stomach from the duodenum (e.g., as determined by checking for recent data stored within ingestible device 65100 at 65612), ingestible device 65100 may select the second subset of data at 65614 from a time frame when ingestible device 65100 is known to be within the stomach. In some embodiments, ingestible device 65100 may alternately select a previously recorded average and standard deviation for ratios of green reflectances and blue reflectances within the stomach (e.g., an average and standard deviation typical of data recorded within the stomach, as previously recorded within memory circuitry of PCB 65120 at 65620) in place of the second subset of data. In this case, ingestible device 65100 may simply use the previously recorded average and previously recorded standard deviation when making a determination at 65616, rather than expending resources to calculate the mean and standard deviation of the second subset.

At 65616, the ingestible device (e.g., ingestible device 65100, 65300, or 65400) determines whether the difference between the mean of the second subset and the mean of the first subset is greater than a predetermined multiple of the standard deviation of the first subset. For example, ingestible device 65100 may compute a difference between a mean of the first subset of recent data and a mean of a second subset of past data, and determine whether this difference is greater than three times the standard deviation of the second subset of past data. In some embodiments, it is understood that any convenient threshold level may be used other than three times the standard deviation, such as any value between one and five times the standard deviation. Also, in some embodiments, the ingestible device may instead set the threshold level based on the standard deviation of the second subset instead of the first subset. In response to determining that the difference between the mean of the first subset and the mean of the second subset is greater than a predetermined multiple of the standard deviation of the second subset, process 65600 proceeds to 65618. Otherwise, process 65600 proceeds back to 65604, where the ingestible device 65604 continues to gather new data to be used in monitoring for transitions between the stomach (e.g., stomach 65306 (FIG. 19)) and the duodenum (e.g., duodenum 65310 (FIG. 19)).

At 65618, the ingestible device (e.g., ingestible device 65100, 65300, or 65400) determines (e.g., via control circuitry within PCB 65120 (FIG. 18)) whether the determination made at 65616 is the first time that the difference between the mean of the first subset of recent data and the mean of the second subset of past data is calculated to be greater than the standard deviation of the second subset. If the ingestible device determines that this is the first time that the difference between the mean of the first subset and the mean of the second subset is calculated to be greater than the standard deviation of the second subset, process 65600 proceeds to 65620 to store the mean of the second subset of past data as an average G/B signal in the stomach. Alternatively, if the ingestible device determines that the immediately preceding determination made at 65616 is not the first time that the difference between the mean of the first subset of recent data and the mean of the second subset of past data is calculated to be greater than the standard deviation of the second subset, process 65600 proceeds directly to 65622.

At 65620, the ingestible device (e.g., ingestible device 65100, 65300, or 65400) stores the mean of the second subset as an average G/B signal in the stomach. For example, ingestible device 65100 may be configured to store the mean of the second subset of past data (e.g., store within memory circuitry of PCB 65120 (FIG. 18)) as the average ratio of the measured green reflectance levels to the measured blue reflectance levels measured in the stomach. In some embodiments, ingestible device 65100 may also store the standard deviation of the second subset of past data as a typical standard deviation of the ratios of the measured green reflectance levels to the measured blue reflectance levels detected within the stomach. This stored information may be used by the ingestible device later on (e.g., at 65610) to compare against future data, which may enable the ingestible device to detect reverse pyloric transitions from the duodenum (e.g., duodenum 65310 (FIG. 19)) back to the stomach (e.g., stomach 65306 (FIG. 19)), and may generally be used in place of other experimental data gathered from the stomach (e.g., in place of the second subset of data at 65616). After storing the mean of the second subset as an average G/B signal in the stomach, process 65600 proceeds to 65622.

At 65622, the ingestible device (e.g., ingestible device 65100, 65300, or 65400) determines whether a difference of the mean of the first subset of recent data to the mean of the second subset of past data is greater than a predetermined threshold, “M”. In some embodiments, the predetermined threshold, “M,” will be sufficiently large to ensure that the mean of the first subset is substantially larger than the mean of the second subset, and may enable ingestible device 65100 to ensure that it detected an actual transition to the duodenum. This may be particularly advantageous when the determination made at 65616 is potentially unreliable due to the standard deviation of the second subset of past data being abnormally small. For example, a typical value of the predetermined threshold “M,” may be on the order of 0.1 to 0.5. If ingestible device 65100 determines that the difference of the mean of the first subset of recent data to the second subset of past data is greater than a predetermined threshold, process 65600 proceeds to 65624 to store data indicating that a pyloric transition from the stomach to the duodenum (e.g., from stomach 65306 to duodenum 65310 (FIG. 19)) was detected. Alternatively, if the ingestible device determines that the ratio of the mean of the first subset to the second subset is less than or equal to the predetermined threshold, “M” (i.e., determines that a transition to the duodenum has not occurred), process 65600 proceeds directly to 65604 where ingestible device 65100 continues to make new measurements and monitor for possible transitions between the stomach and the duodenum.

In some embodiments, instead of using a difference of the mean of the first subset of recent data to the mean of the second subset of past data, the ingestible device (e.g., ingestible device 65100, 65300, or 65400) determines whether the ratio of the mean of the first subset of recent data to the mean of the second subset of past data is greater than a predetermined threshold, “M”. In some embodiments, the predetermined threshold, “M,” will be sufficiently large to ensure that the mean of the first subset is substantially larger than the mean of the second subset, and may enable ingestible device 65100 to ensure that it detected an actual transition to the duodenum. This may be particularly advantageous when the determination made at 65616 is potentially unreliable due to the standard deviation of the second subset of past data being abnormally small. For example, a typical value of the predetermined threshold “M,” may be on the order of 1.2 to 2.0. It is understood any convenient type of threshold or calculation may be used to determine whether or not the first subset of data and the second subset of data are both statistically distinct from one another, and also substantially different from one another in terms of overall average value.

At 65624, the ingestible device (e.g., ingestible device 65100, 65300, or 65400) stores data indicating a pyloric transition from the stomach to the duodenum was detected. For example, ingestible device 65100 may store a data flag (e.g., within memory circuitry of PCB 65120 (FIG. 18)) indicating that the ingestible device 65100 most recently detected itself to be within the duodenum portion of the GI tract (e.g., duodenum 65310 (FIG. 19)). In some embodiments, ingestible device 65100 may also store data (e.g., within memory circuitry of PCB 65120 (FIG. 18)) indicating a time that ingestible device 65100 detected the pyloric transition from the stomach to the duodenum. This information may be used by ingestible device 65100 at 65608, and as a result process 65600 may proceed from 65608 to 65610, rather than proceeding from 65618 to 65614. After ingestible device 65100 stores the data indicating a pyloric transition from the stomach to the duodenum was detected, process 65600 proceeds to 65604 where ingestible device 65100 continues to gather additional measurements, and continues to monitor for further transitions between the stomach and the duodenum.

It will be understood that the steps and descriptions of the flowcharts of this disclosure, including FIG. 22, are merely illustrative. Any of the steps and descriptions of the flowcharts, including FIG. 22, may be modified, omitted, rearranged, and performed in alternate orders or in parallel, two or more of the steps may be combined, or any additional steps may be added, without departing from the scope of the present disclosure. For example, the ingestible device 65100 may calculate the mean and the standard deviation of multiple data sets in parallel in order to speed up the overall computation time. Furthermore, it should be noted that the steps and descriptions of FIG. 22 may be combined with any other system, device, or method described in this application, and any of the ingestible devices or systems discussed in this application could be used to perform one or more of the steps in FIG. 22. For example, portions of process 65600 may be incorporated into 65508-65516 of process 65500 (FIG. 21), and may be part of a more general process for determining a location of the ingestible device. As another example, the ratio of detected blue and green light (e.g., as measured and added to the data set at 65604) may continue even outside of the stomach or duodenum, and similar information may be recorded by the ingestible device throughout its transit in the GI tract. Example plots of data sets of ratios of measured green and blue reflectance levels, which may be gathered throughout the GI tract, are discussed further in relation to FIG. 23 and FIG. 24 below.

FIG. 23 is a plot illustrating data collected during an example operation of an ingestible device (e.g., ingestible device 65100, 65300, or 65400), which may be used when determining a location of an ingestible device as it transits through a gastrointestinal (GI) tract, in accordance with some embodiments of the disclosure. Although FIG. 23 may be described in connection with ingestible device 65100 for illustrative purposes, this is not intended to be limiting, and plot 65700 and data set 65702 may be typical of data gathered by any device discussed in this application. Plot 65700 depicts the ratios of the measured green reflectance levels to the measured blue reflectance levels over time. For example, ingestible device 65100 may have computed the value for each point in the data set 65702 by transmitting green and blue illumination at a given time (e.g., via illuminator 65124 (FIG. 18)), measuring the resulting green and blue reflectances (e.g., via detector 65122 (FIG. 18)), calculating the ratio of the resulting reflectances, and storing the ratio in the data set along with a timestamp indicating the time that the reflectances were gathered.

At 65704, shortly after ingestible device 65100 begins operation, ingestible device 65100 determines that it has reached at least the stomach (e.g., as a result of making a determination similar to the determination discussed in relation to 65506 in process 65500 (FIG. 21)). Ingestible device 65100 continues to gather additional measurements of green and blue reflectance levels, and at 65706 ingestible device 65100 determines that a pyloric transition has occurred from the stomach to the duodenum (e.g., as a result of making a determination similar to the determinations discussed in relation to 65616-65624 of process 65600 (FIG. 22)). Notably, the values in data set 65702 around 65706 jump up precipitously, which is indicative of the higher ratios of measured green reflectance levels to measured blue reflectance levels typical of the duodenum.

The remainder of the data set 65702 depicts the ratios of the measured green reflectance levels to the measured blue reflectance levels throughout the remainder of the GI tract. At 65708, ingestible device 65100 has reached the jejunum (e.g., as determined through measurements of muscle contractions, as discussed in relation to FIG. 25), and by 65710, ingestible device 65100 has reached the cecum. It is understood that, in some embodiments, the overall character and appearance of data set 65702 changes within the small intestine (i.e., the duodenum, jejunum, and ileum) versus the cecum. Within the jejunum and ileum, there may typically be a wide variation in the ratios of the measured green reflectance levels to the measured blue reflectance levels, resulting in relatively noisy data with a high standard deviation. By comparison, within the cecum ingestible device 65100 may measure a relatively stable ratio of the measured green reflectance levels to the measured blue reflectance levels. In some embodiments, ingestible device 65100 may be configured to determine transitions from the small intestine to the cecum based on these differences. For example, ingestible device 65100 may compare recent windows of data to past windows of data, and detect a transition to the cecum in response to determining that the standard deviation of the ratios in the recent window of data is substantially less than the standard deviation of the ratios in the past window of data.

FIG. 24 is another plot illustrating data collected during an example operation of an ingestible device, which may be used when determining a location of an ingestible device as it transits through a gastrointestinal (GI) tract, in accordance with some embodiments of the disclosure. Similar to FIG. 23, FIG. 24 may be described in connection with the ingestible device 65100 for illustrative purposes. However, this is not intended to be limiting, and plot 65800 and data set 65802 may be typical of data gathered by any device discussed in this application.

At 65804, shortly after ingestible device 65100 begins operation, ingestible device 65100 determines that it has reached at least the stomach (e.g., as a result of making a determination similar to the determination discussed in relation to 65506 in process 500 (FIG. 21)). Ingestible device 65100 continues to gather additional measurements of green and blue reflectance levels (e.g., via sensing sub-unit 65126 (FIG. 18)), and at 65806 ingestible device 65100 determines that a pyloric transition has occurred from the stomach to the duodenum (e.g., as a result of making a determination similar to the determinations discussed in relation to 65616-65624 of process 65600 (FIG. 22)). Notably, the values in data set 65802 around 65806 jump up precipitously, which is indicative of the higher ratios of measured green reflectance levels to measured blue reflectance levels typical of the duodenum, before falling shortly thereafter. As a result of the reduced values in data set 65802, ingestible device 65100 determines that a reverse pyloric transition has occurred from the duodenum back to the stomach at 65808 (e.g., as a result of making a determination similar to the determinations discussed in relation to 65610-65612 of process 65600 (FIG. 22)). At 65810, as a result of the values in data set 65802 increasing again, ingestible device 65100 determines that another pyloric transition has occurred from the stomach to the duodenum, and shortly thereafter ingestible device 65100 proceeds onwards to the jejunum, ileum, and cecum.

The remainder of the data set 65802 depicts the ratios of the measured green reflectance levels to the measured blue reflectance levels throughout the remainder of the GI tract. Notably, at 65812, ingestible device reaches the transition point between the ileum and the cecum. As discussed above in relation to FIG. 23, the transition to the cecum is marked by a reduced standard deviation in the ratios of measured green reflectances and measured blue reflectances over time, and ingestible device 65100 may be configured to detect a transition to the cecum based on determining that the standard deviation of a recent set of measurements is substantially smaller than the standard deviation of past measurements taken from the jejunum or ileum.

FIG. 25 is a flowchart of illustrative steps for detecting a transition from a duodenum to a jejunum, which may be used when determining a location of an ingestible device as it transits through a gastrointestinal (GI) tract, in accordance with some embodiments of the disclosure. Although FIG. 25 may be described in connection with the ingestible device 65100 for illustrative purposes, this is not intended to be limiting, and either portions or the entirety of process 65900 described in FIG. 25 may be applied to any device discussed in this application (e.g., the ingestible devices 65100, 65300, and 65400), and any of these ingestible devices may be used to perform one or more parts of the process described in FIG. 25. Furthermore, the features of FIG. 25 may be combined with any other systems, methods or processes described in this application. For example, portions of the process described by the process in FIG. 25 may be integrated into the localization process described by FIG. 21 (e.g., as part of 65520-65524 of process 65500 (FIG. 21)). In some embodiments, an ingestible device 65100 may perform process 65900 while in the duodenum, or in response to detecting entry to the duodenum. In other embodiments, an ingestible device 65100 may perform process 65900 while in the stomach, or in response to detecting entry into the GI tract. It is also understood that process 65900 may be performed in parallel with any other process described in this disclosure (e.g., process 65600 (FIG. 22)), which may enable ingestible device 65100 to detect entry into various portions of the GI tract, without necessarily detecting entry into a preceding portion of the GI tract.

For illustrative purposes, FIG. 25 may be discussed in terms of ingestible device 65100 generating and making determinations based on a single set of reflectance levels generated at a single wavelength by a single sensing sub-unit (e.g., sensing sub-unit 65126 (FIG. 18)). However, it is understood that ingestible device 65100 may generate multiple wavelengths of illumination from multiple different sensing sub-units positioned around the circumference of ingestible device (e.g., multiple sensing sub-units positioned at different locations behind window 65114 of ingestible device 65100 (FIG. 17), and each of the resulting reflectances may be stored as a separate data set. Moreover, each of these sets of reflectance levels may be used to detect muscle contractions by running multiple versions of process 65900, each one of which processes data for a different set of reflectances corresponding to data sets obtained from measurements of different wavelengths or measurements made by different sensing sub-units.

At 65902, the ingestible device (e.g., ingestible device 65100, 65300, or 65400) retrieves a set of reflectance levels. For example, ingestible device 65100 may retrieve a data set of previously recorded reflectance levels from memory (e.g., from memory circuitry of PCB 65120 (FIG. 18)). Each of the reflectance levels may correspond to reflectances previously detected by ingestible device 65100 (e.g., via detector 65122 (FIG. 18)) from illumination generated by ingestible device 65100 (e.g., via illuminator 65124 (FIG. 18)), and may represent a value indicative of an amount of light detected in a given reflectance. However, it is understood that any suitable frequency of light may be used, such as light in the infrared, visible, or ultraviolet spectrums. In some embodiments, the reflectance levels may correspond to reflectances previously detected by ingestible device 65100 at periodic intervals.

At 904, the ingestible device (e.g., ingestible device 65100, 65300, or 65400) includes new measurements of reflectance levels in the data set. For example, ingestible device 65100 may be configured to detect a new reflectance (e.g., transmit illumination and detect the resulting reflectance using sensing sub-unit 65126 (FIG. 18)) at regular intervals, or with sufficient speed as to detect peristaltic waves. For example, ingestible device 65100 may be configured to generate illumination and measure the resulting reflectance once every three seconds (i.e., potentially minimum rate to detect a 0.17 Hz signal), and preferably at a higher rate, as fast at 0.1 second or even faster. It is understood that the periodic interval between measurements may be adapted as needed based on the species of the subject, and the expected frequency of the peristaltic waves to be measured. Every time ingestible device 65100 makes a new reflectance level measurement at 65904, the new data is included to the data set (e.g., a data set stored within memory circuitry of PCB 65120 (FIG. 18)).

At 65906, the ingestible device (e.g., ingestible device 65100, 65300, or 65400) obtains a first subset of recent data by applying a sliding window filter to the data set. For example, ingestible device 65100 may retrieve a one-minute worth of data from the data set. If the data set includes values for reflectances measured every second, this would be approximately 60 data points worth of data. Any suitable type of window size may be used, provided that the size of the window is sufficiently large to detect peristaltic waves (e.g., fluctuations on the order of 0.1 Hz to 0.2 Hz for healthy human subjects). In some embodiments, ingestible device 65100 may also clean the data, for example, by removing outliers from the first subset of data obtained through the use of the sliding window filter.

At 65908, the ingestible device (e.g., ingestible device 65100, 65300, or 65400) obtains a second subset of recent data by interpolating the first subset of recent data. For example, ingestible device 65100 may interpolate the first subset of data in order to generate a second subset of data with a sufficient number of data points (e.g., data points spaced every 0.5 seconds or greater). In some embodiments, this may enable ingestible device 65100 to also replace any outlier data points that may have been removed as part of applying the window filter at 65906.

At 65910, the ingestible device (e.g., ingestible device 65100, 65300, or 65400) calculates a normalized frequency spectrum from the second subset of data. For example, ingestible device 65100 may be configured to perform a fast Fourier transform to convert the second subset of data from a time domain representation into a frequency domain representation. It is understood that depending on the application being used, and the nature of the subset of data, any number of suitable procedures (e.g., Fourier transform procedures) may be used to determine a frequency spectrum for the second subset of data. For example, the sampling frequency and size of the second subset of data may be known in advance, and ingestible device 65100 may be configured to have pre-stored values of a normalized discreet Fourier transform (DFT) matrix, or the rows of the DFT matrix corresponding to the 0.1 Hz to 0.2 Hz frequency components of interest, within memory (e.g., memory circuitry of PCB 65120 (FIG. 18)). In this case, the ingestible device may use matrix multiplication between the DFT matrix and the data set to generate an appropriate frequency spectrum. An example data set and corresponding frequency spectrum that may be obtained by the ingestible device is discussed in greater detail in relation to FIG. 26.

At 65912, the ingestible device (e.g., ingestible device 65100, 65300, or 65400) determines whether at least a portion of the normalized frequency spectrum is between 0.1 Hz and 0.2 Hz above a threshold value of 0.5 Hz. Peristaltic waves in a healthy human subject occur at a rate between 0.1 Hz and 0.2 Hz, and an ingestible device experiencing peristaltic waves (e.g., ingestible device 65400 detecting contractions in walls 65406 of the jejunum (FIG. 20)) may detect sinusoidal variations in the amplitude of detected reflectances levels that follow a similar 0.1 Hz to 0.2 Hz frequency. If the ingestible device determines that a portion of the normalized frequency spectrum between 0.1 Hz and 0.2 Hz is above a threshold value of 0.5, this measurement may be consistent with peristaltic waves in a healthy human subject, and process 65900 proceeds to 65914 where ingestible device 65100 stores data indicating a muscle contraction was detected. Alternatively, if the ingestible device determines that no portion of the normalized frequency spectrum between 0.1 Hz and 0.2 Hz above a threshold value of 0.5, process 65900 proceeds directly to 65904 to make new measurements and to continue to monitor for new muscle contractions. It is understood that a threshold value other than 0.5 may be used, and that the exact threshold may depend on the sampling frequency and type of frequency spectrum used by ingestible device 65100.

At 65914, the ingestible device (e.g., ingestible device 65100, 65300, or 65400) stores data indicating a muscle contraction was detected. For example, ingestible device 65100 may store data in memory (e.g., memory circuitry of PCB 65120 (FIG. 18)) indicating that a muscle contraction was detected, and indicating the time that the muscle contraction was detected. In some embodiments, ingestible device 65100 may also monitor the total number of muscle contractions detected, or the number of muscle contractions detected in a given time frame. In some embodiments, detecting a particular number of muscle contractions may be consistent with ingestible device 65100 being within the jejunum (e.g., jejunum 65314 (FIG. 19)) of a healthy human subject. After detecting a muscle contraction, process 65900 proceeds to 65916.

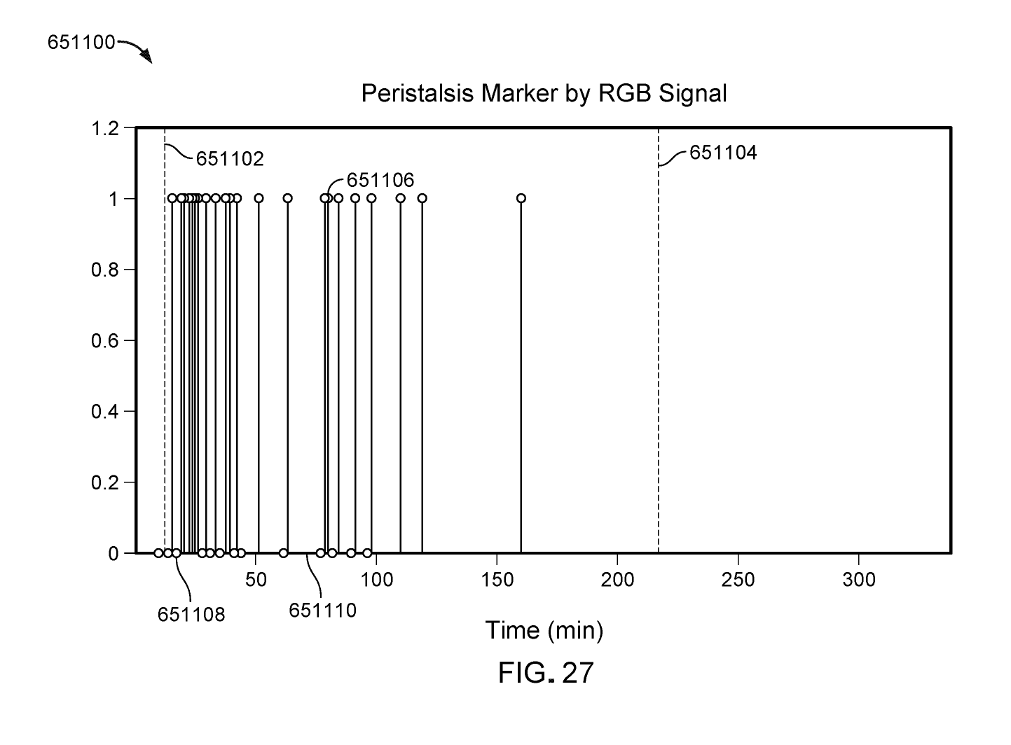

At 65916, the ingestible device (e.g., ingestible device 65100, 65300, or 65400) determines whether a total number of muscle contractions exceeds a predetermined threshold number. For example, ingestible device 65100 may retrieve the total number of muscle contractions detected from memory (e.g., from memory circuitry of PCB 65120 (FIG. 18)), and compare the total number to a threshold value. In some embodiments, the threshold value may be one, or any number larger than one. The larger the threshold value, the more muscle contractions need to be detected before ingestible device 65100 stores data indicating that it has entered the jejunum. In practice, setting the threshold value as three or higher may prevent the ingestible device from detecting false positives (e.g., due to natural movement of the GI tract organs, or due to movement of the subject). If the total number of contractions exceeds the predetermined threshold number, process 65900 proceeds to 65918 to store data indicating detection of a transition from the duodenum to the jejunum. Alternatively, if the total number of contractions does not exceed a predetermined threshold number, process 65900 proceeds to 65904 to include new measurements of reflectance levels in the data set. An example plot of the muscle contractions detected over time is discussed in greater detail in relation to FIG. 27.

At 65918, the ingestible device (e.g., ingestible device 65100, 65300, or 65400) stores data indicating detection of a transition from the duodenum to the jejunum. For example, ingestible device 65100 may store data in memory (e.g., from memory circuitry of PCB 65120 (FIG. 18)) indicating that the jejunum has been reached. In some embodiments, if ingestible device 65100 is configured to perform all or part of process 65900 while in the stomach, ingestible device 65100 may store data at 65918 indicating detection of a transition from the stomach directly to the jejunum (e.g., as a result of transitioning too quickly through the duodenum for the pyloric transition to be detected using process 65600 (FIG. 22)).

In some embodiments, the ingestible device (e.g., ingestible device 65100, 65300, or 65400) may be configured to obtain a fluid sample from the environment external to a housing of the ingestible device in response to identifying a change in the location of the ingestible device. For example, ingestible device 65100 may be configured to obtain a fluid sample from the environment external to the housing of ingestible device 65100 (e.g., through the use of optional opening 65116 and optional rotating assembly 65118 (FIG. 18)) in response to determining that the ingestible device is located within the jejunum (e.g., jejunum 65314 (FIG. 19)). In some embodiments, ingestible device 65100 may also be equipped with appropriate diagnostics to detect certain medical conditions based on the retrieved fluid sample, such as small intestinal bacterial overgrowth (SIBO).

In some embodiments, the ingestible device (e.g., ingestible device 65100, 65300, or 65400) may be configured to deliver a dispensable substance that is pre-stored within the ingestible device from the ingestible device into the gastrointestinal tract in response to identifying the change in the location of the ingestible device. For example, ingestible device 65100 may have a dispensable substance pre-stored within the ingestible device 65100 (e.g., within a storage chamber or cavity on optional storage sub-unit 65118-3 (FIG. 18)), and ingestible device 65100 may be configured to dispense the substance into the gastrointestinal tract (e.g., through the use of optional opening 65116 and optional rotating assembly 65118 (FIG. 18)) when the ingestible device 65100 detects that the ingestible device 65100 is located within the jejunum (e.g., jejunum 65314 (FIG. 19)). In some embodiments, this may enable ingestible device 65100 to deliver substances (e.g., therapeutics and medicaments) at targeted locations within the GI tract.

In some embodiments, the ingestible device (e.g., ingestible device 65100, 65300, or 65400) may be configured to perform an action based on the total number of detected muscle contractions. For example, ingestible device 65100 may be configured to retrieve data indicative of the total number of muscle contractions (e.g., from memory circuitry of PCB 65120 (FIG. 18)), and compare that to an expected number of muscle contractions in a healthy individual. In response, the ingestible device may either dispense a substance into the gastrointestinal tract (e.g., through the use of optional opening 65116 and optional rotating assembly 65118 (FIG. 66)), or may obtain a fluid sample from the environment external to the housing of ingestible device 65100 (e.g., through the use of optional opening 65116 and optional rotating assembly 65118 (FIG. 18)). For instance, ingestible device 65100 may be configured to obtain a sample in response to determining that a number of detected muscle contractions is abnormal, and differs greatly from the expected number. As another example, ingestible device 65100 may be configured to deliver a substance into the GI tract (such as a medicament), in response to determining that the detected muscle contractions are consistent with a functioning GI tract in a healthy individual.

It will be understood that the steps and descriptions of the flowcharts of this disclosure, including FIG. 25, are merely illustrative. Any of the steps and descriptions of the flowcharts, including FIG. 25, may be modified, omitted, rearranged, performed in alternate orders or in parallel, two or more of the steps may be combined, or any additional steps may be added, without departing from the scope of the present disclosure. For example, the ingestible device 65100 may calculate the mean and the standard deviation of multiple data sets in parallel (e.g., multiple data sets, each one corresponding to a different wavelength of reflectance or different sensing sub-unit used to detect the reflectance) in order to speed up the overall computation time. Furthermore, it should be noted that the steps and descriptions of FIG. 25 may be combined with any other system, device, or method described in this application, and any of the ingestible devices or systems discussed in this application could be used to perform one or more of the steps in FIG. 25.

FIG. 26 is a plot illustrating data collected during an example operation of an ingestible device, which may be used when detecting a transition from a duodenum to a jejunum, in accordance with some embodiments of the disclosure. Diagram 651000 depicts a time domain plot 651002 of a data set of reflectance levels measured by an ingestible device (e.g., the second subset of data discussed in relation to 65908 of FIG. 25). In some embodiments, ingestible device 65100 may be configured to gather data points at semi-regular intervals approximately 0.5 seconds apart. By comparison, diagram 651050 depicts a frequency domain plot 651004 of the same data set of reflectance levels measured by an ingestible device (e.g., as a result of ingestible device 65100 calculating a frequency spectrum at 65910 of FIG. 25). In some embodiments, ingestible device 65100 may be configured to calculate the frequency spectrum through any convenient means.

In diagram 651050, the range of frequencies 651006 between 0.1 Hz and 0.2 Hz may be the range of frequencies that ingestible device 65100 searches in order to detect muscle contractions. As shown in diagram 651050, there is a strong peak in the frequency domain plot 651004 around 0.14 Hz, which is consistent with the frequency of peristaltic motion in a healthy human individual. In this case, an ingestible device 65100 analyzing frequency domain plot 651004 may be configured to determine that the data is consistent with a detected muscle contraction (e.g., using a process similar to 65912 of process 65900 (FIG. 25)), and may store data (e.g., in memory circuitry of PCB 65120 (FIG. 18)) indicating that a muscle contraction has been detected. Because the muscle contraction was detected from the one-minute window of data ending at 118 minutes, ingestible device 65100 may also store data indicating that the muscle contraction was detected at the 118-minute mark (i.e., which may indicate that the ingestible device 65100 was turned on and ingested by the subject 118 minutes ago).

FIG. 27 is a plot illustrating muscle contractions detected by an ingestible device over time, which may be used when determining a location of an ingestible device as it transits through a gastrointestinal (GI) tract, in accordance with some embodiments of the disclosure. In some embodiments, ingestible device 65100 may be configured to detect muscle contractions, and store data indicative of when each muscle contraction is detected (e.g., as part of 65914 of process 65900 (FIG. 25)). Plot 651100 depicts the detected muscle contractions 651106 over time, with each muscle contraction being represented by a vertical line reaching from “0” to “1” on the y-axis.

At 651102, around the 10-minute mark, ingestible device 65100 first enters the duodenum (e.g., as determined by ingestible device 65100 performing process 65600 (FIG. 22)). Shortly thereafter, at 651108, ingestible device 65100 begins to detect several muscle contractions 1106 in quick succession, which may be indicative of the strong peristaltic waves that form in the jejunum (e.g., jejunum 65314 (FIG. 19)). Later, around 651110, ingestible device 65100 continues to detect intermittent muscle contractions, which may be consistent with an ingestible device 65100 within the ileum. Finally, at 651104, ingestible device 65100 transitions out of the small intestine, and into the cecum. Notably, ingestible device 65100 detects more frequent muscle contractions in the jejunum portion of the small intestine as compared to the ileum portion of the small intestine, and ingestible device 65100 does not measure any muscle contractions after having exited the small intestine. In some embodiments, ingestible device 65100 may incorporate this information into a localization process. For example, ingestible device 65100 may be configured to detect a transition from a jejunum to an ileum in response to determining that a frequency of detected muscle contractions (e.g., the number of muscle contractions measured in a given 10-minute window) has fallen below a threshold number. As another example, ingestible device 65100 may be configured to detect a transition from an ileum to a cecum in response to determining that no muscle contractions have been detected for a threshold period of time. It is understood that these examples are intended to be illustrative, and not limiting, and that measurements of muscle contractions may be combined with any of the other processes, systems, or methods discussed in this disclosure.

FIG. 27 is a flowchart 651200 for certain embodiments for determining a transition of the device from the jejunum to the ileum. It is to be noted that, in general, the jejunum is redder and more vascular than the ileum. Moreover, generally, in comparison to the ileum, the jejunum has a thicker intestine wall with more messentary fat. These differences between the jejunum and the ileum are expected to result in differences in optical responses in the jejunum relative to the ileum. Optionally, one or more optical signals may be used to investigate the differences in optical responses. For example, the process can include monitoring a change in optical response in reflected red light, blue light, green light, ratio of red light to green light, ratio of red light to blue light, and/or ratio of green light to blue light. In some embodiments, reflected red light is detected in the process.

Flowchart 651200 represents a single sliding window process. In step 651210, the jejenum reference signal is determined based on optical reflection. Typically, this signal is as the average signal (e.g., reflected red light) over a period of time since the device was determined to enter the jejenum. The period of time can be, for example, from five minutes to 40 minutes (e.g., from 10 minutes to 30 minutes, from 15 minutes to 25 minutes). In step 651220, the detected signal (e.g., reflected red light) just after the period of time used in step 651210 is normalized to the reference signal determined in step 651210. In step 651230, the signal (e.g., reflected red light) is detected. In step 651240, the mean signal detected based on the single sliding window is compared to a signal threshold. The signal threshold in step 651240 is generally a fraction of the reference signal of the jejenum reference signal determined in step 651210. For example, the signal threshold can be from 60% to 90% (e.g., from 70% to 80%) of the jejenum reference signal. If the mean signal exceeds the signal threshold, then the process determines that the device has entered the ileum at step 651250. If the mean signal does not exceed the signal threshold, then the process returns to step 651230.

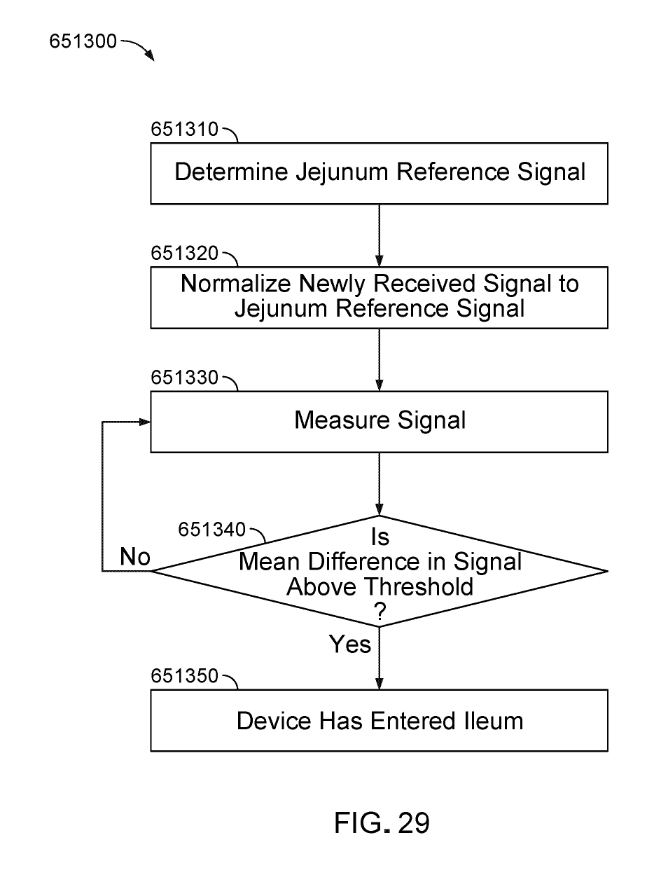

FIG. 29 is a flowchart 651200 for certain embodiments for determining a transition of the device from the jejunum to the ileum using a two sliding window process. In step 651310, the jejenum reference signal is determined based on optical reflection. Typically, this signal is as the average signal (e.g., reflected red light) over a period of time since the device was determined to enter the jejenum. The period of time can be, for example, from five minutes to 40 minutes (e.g., from 10 minutes to 30 minutes, from 15 minutes to 25 minutes). In step 651320, the detected signal (e.g., reflected red light) just after the period of time used in step 651310 is normalized to the reference signal determined in step 651310. In step 651330, the signal (e.g., reflected red light) is detected. In step 651340, the mean difference in the signal detected based on the two sliding windows is compared to a signal threshold. The signal threshold in step 651340 is based on whether the mean difference in the detected signal exceeds a multiple (e.g., from 1.5 times to five times, from two times to four times) of the detected signal of the first window. If signal threshold is exceeded, then the process determines that the device has entered the ileum at step 651350. If the signal threshold is not exceeded, then the process returns to step 651330.

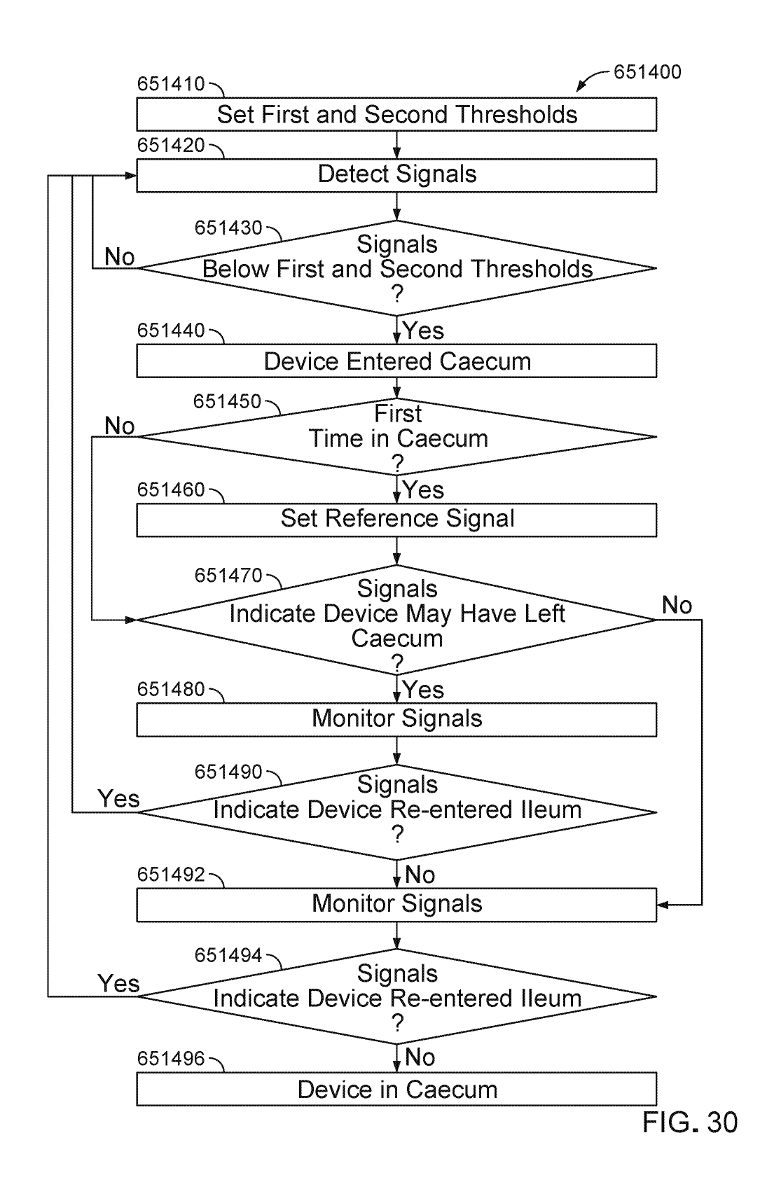

FIG. 30 is a flowchart 651400 for a process for certain embodiments for determining a transition of the device from the ileum to the cecum. In general, the process involves detecting changes in the reflected optical signal (e.g., red light, blue light, green light, ratio of red light to green light, ratio of red light to blue light, and/or ratio of green light to blue light). In some embodiments, the process includes detecting changes in the ratio of reflected red light to reflected green light, and also detecting changes in the ratio of reflected green light to reflected blue light. Generally, in the process 651400, the sliding window analysis (first and second windows) discussed with respect to process 65600 is continued.

Step 651410 includes setting a first threshold in a detected signal, e.g., ratio of detected red light to detected green light, and setting a second threshold for the coefficient of variation for a detected signal, e.g., the coefficient of variation for the ratio of detected green light to detected blue light. The first threshold can be set to a fraction (e.g., from 0.5 to 0.9, from 0.6 to 0.8) of the average signal (e.g., ratio of detected red light to detected green light) in the first window, or a fraction (e.g., from 0.4 to 0.8, from 0.5 to 0.7) of the mean difference between the detected signal (e.g., ratio of detected red light to detected green light) in the two windows. The second threshold can be set to 0.1 (e.g., 0.05, 0.02).

Step 651420 includes detecting the signals in the first and second windows that are to be used for comparing to the first and second thresholds.

Step 651430 includes comparing the detected signals to the first and second thresholds. If the corresponding value is not below the first threshold or the corresponding value is not below the second threshold, then it is determined that the device has not left the ileum and entered the cecum, and the process returns to step 651420. If the corresponding value is below the first threshold and the corresponding value is below the second threshold, then it is determined that the device has left the ileum and entered the cecum, and the proceeds to step 651440.

Step 651450 includes determining whether it is the first time that that the device was determined to leave the ileum and enter the cecum. If it is the first time that the device was determined to leave the ileum and enter the cecum, then the process proceeds to step 651460. If it is not the first time that the device has left the ileum and entered the cecum, then the process proceeds to step 651470.

Step 651460 includes setting a reference signal. In this step the optical signal (e.g., ratio of detected red light to detected green light) as a reference signal.

Step 651470 includes determining whether the device may have left the cecum and returned to the ileum. The device is determined to have left the cecum and returned to the ileum if the corresponding detected signal (e.g., ratio of detected red light to detected green light) is statistically comparable to the reference signal (determined in step 651460) and the coefficient of variation for the corresponding detected signal (e.g., ratio of detected green light to detected blue light) exceeds the second threshold. If it is determined that the device may have left the cecum and returned to the ileum, the process proceeds to step 651480.

Step 651480 includes continuing to detect the relevant optical signals for a period of time (e.g., at least one minute, from five minutes to 15 minutes).

Step 651490 includes determining whether the signals determined in step 651480 indicate (using the methodology discussed in step 651470) that the device re-entered the ileum. If the signals indicate that the device re-entered the ileum, the process proceeds to step 651420. If the signals indicate that the device is in the cecum, the process proceeds to step 651492.

Step 651492 includes continuing to monitor the relevant optical signals for a period of time (e.g., at least 30 minutes, at least one hour, at least two hours).

Step 651494 includes determining whether the signals determined in step 651492 indicate (using the methodology discussed in step 651470) that the device re-entered the ileum. If the signals indicate that the device re-entered the ileum, the process proceeds to step 651420. If the signals indicate that the device is in the cecum, the process proceeds to step 651496.

At step 651496, the process determines that the device is in the cecum.

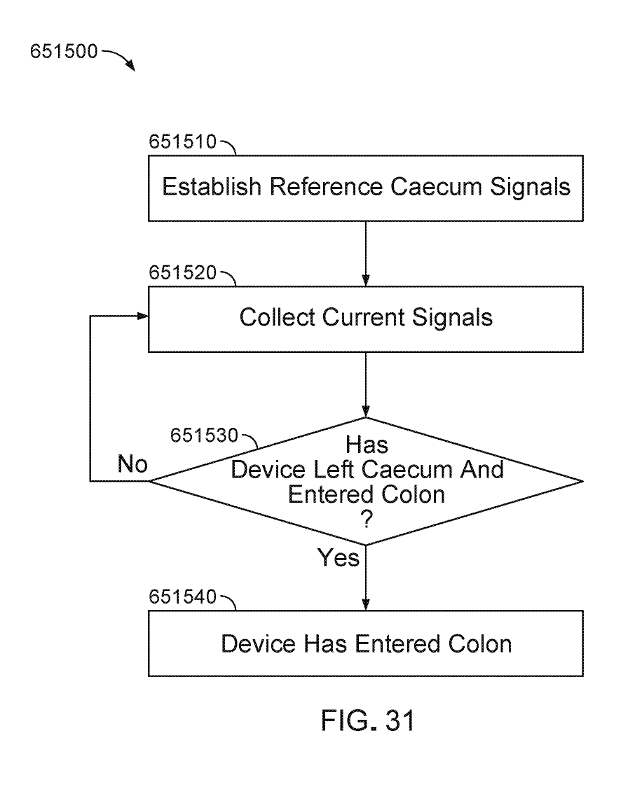

FIG. 31 is a flowchart 651500 for a process for certain embodiments for determining a transition of the device from the cecum to the colon. In general, the process involves detecting changes in the reflected optical signal (e.g., red light, blue light, green light, ratio of red light to green light, ratio of red light to blue light, and/or ratio of green light to blue light). In some embodiments, the process includes detecting changes in the ratio of reflected red light to reflected green light, and also detecting changes in the ratio of reflected blue light. Generally, in the process 651500, the sliding window analysis (first and second windows) discussed with respect to process 651400 is continued.

In step 651510, optical signals (e.g., the ratio of reflected red signal to reflected green signal, and reflected blue signal) are collected for a period of time (e.g., at least one minute, at least five minutes, at least 10 minutes) while the device is in the cecum (e.g., during step 651480). The average values for the recorded optical signals (e.g., the ratio of reflected red signal to reflected green signal, and reflected blue signal) establish the cecum reference signals.

In step 651520, the optical signals are detected after it has been determined that the device entered the cecum (e.g., at step 651440). The optical signals are normalized to the cecum reference signals.

Step 651530 involves determining whether the device has entered the colon. This includes determining whether any of three different criteria are satisfied. The first criterion is satisfied if the mean difference in the ratio of a detected optical signal (e.g., ratio of detected red signal to the detected green) is a multiple greater than one (e.g., 2×, 3×, 4×) the standard deviation of the corresponding signal (e.g., ratio of detected red signal to the detected green) in the second window. The second criterion is satisfied if the mean of a detected optical signal (e.g., a ratio of detected red light to detected green light) exceeds a given value (e.g., exceeds one). The third criterion is satisfied if the coefficient of variation of an optical signal (e.g., detected blue light) in the first window exceeds a given value (e.g., exceeds 0.2). If any of the three criteria are satisfied, then the process proceeds to step 651540. Otherwise, none of the three criteria are satisfied, the process returns to step 651520.

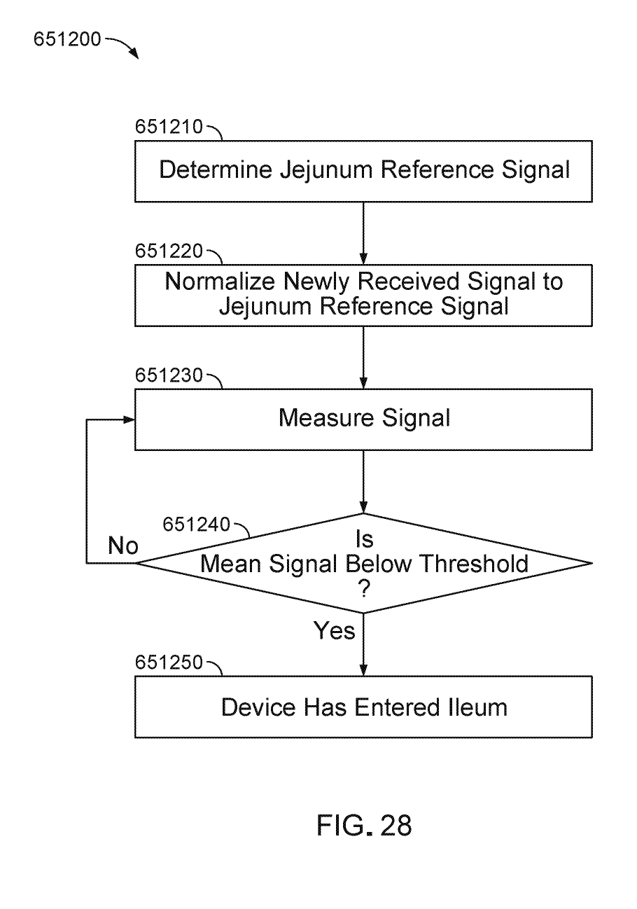

For illustrative purposes the disclosure focuses primarily on a number of different example embodiments of an ingestible device, and example embodiments of methods for determining a location of an ingestible device within a GI tract. However, the possible ingestible devices that may be constructed are not limited to these embodiments, and variations in the shape and design may be made without significantly changing the functions and operations of the device. Similarly, the possible procedures for determining a location of the ingestible device within the GI tract are not limited to the specific procedures and embodiments discussed (e.g., process 65500 (FIG. 21), process 65600 (FIG. 22), process 65900 (FIG. 25), process 651200 (FIG. 28), process 651300 (FIG. 29), process 651400 (FIG. 30) and process 651500 (FIG. 31)). Also, the applications of the ingestible devices described herein are not limited merely to gathering data, sampling and testing portions of the gastrointestinal tract, or delivering medicament. For example, in some embodiments the ingestible device may be adapted to include a number of chemical, electrical, or optical diagnostics for diagnosing a number of diseases. Similarly, a number of different sensors for measuring bodily phenomenon or other physiological qualities may be included on the ingestible device. For example, the ingestible device may be adapted to measure elevated levels of certain chemical compounds or impurities in the gastrointestinal tract, or the combination of localization, sampling, and appropriate diagnostic and assay techniques incorporated into a sampling chamber may be particularly well suited to determine the presence of small intestinal bacterial overgrowth (SIBO).

At least some of the elements of the various embodiments of the ingestible device described herein that are implemented via software (e.g., software executed by control circuitry within PCB 65120 (FIG. 18)) may be written in a high-level procedural language such as object oriented programming, a scripting language or both. Accordingly, the program code may be written in C, C⁺⁺ or any other suitable programming language and may comprise modules or classes, as is known to those skilled in object oriented programming. Alternatively, or in addition, at least some of the elements of the embodiments of the ingestible device described herein that are implemented via software may be written in assembly language, machine language or firmware as needed. In either case, the language may be a compiled or an interpreted language.

At least some of the program code used to implement the ingestible device can be stored on a storage media or on a computer readable medium that is readable by a general or special purpose programmable computing device having a processor, an operating system and the associated hardware and software to implement the functionality of at least one of the embodiments described herein. The program code, when read by the computing device, configures the computing device to operate in a new, specific and predefined manner in order to perform at least one of the methods described herein.

Furthermore, at least some of the programs associated with the systems, devices, and methods of the example embodiments described herein are capable of being distributed in a computer program product comprising a computer readable medium that bears computer usable instructions for one or more processors. The medium may be provided in various forms, including non-transitory forms such as, but not limited to, one or more discettes, compact discs, tapes, chips, and magnetic and electronic storage. In some embodiments, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

The techniques described above can be implemented using software for execution on a computer. For instance, the software forms procedures in one or more computer programs that execute on one or more programmed or programmable computer systems (which may be of various architectures such as distributed, client/server, or grid) each including at least one processor, at least one data storage system (including volatile and non-volatile memory and/or storage elements), at least one input device or port, and at least one output device or port.

The software may be provided on a storage medium, such as a CD-ROM, readable by a general or special purpose programmable computer or delivered (encoded in a propagated signal) over a communication medium of a network to the computer where it is executed. All of the functions may be performed on a special purpose computer, or using special-purpose hardware, such as coprocessors. The software may be implemented in a distributed manner in which different parts of the computation specified by the software are performed by different computers. Each such computer program is preferably stored on or downloaded to a storage media or device (e.g., solid state memory or media, or magnetic or optical media) readable by a general or special purpose programmable computer, for configuring and operating the computer when the storage media or device is read by the computer system to perform the procedures described herein. The inventive system may also be considered to be implemented as a computer-readable storage medium, configured with a computer program, where the storage medium so configured causes a computer system to operate in a specific and predefined manner to perform the functions described herein.

For illustrative purposes the examples given herein focus primarily on a number of different example embodiments of an ingestible device. However, the possible ingestible devices that may be constructed are not limited to these embodiments, and variations in the general shape and design may be made without significantly changing the functions and operations of the device. For example, some embodiments of the ingestible device may feature a sampling chamber substantially towards the middle of the device, along with two sets of axial sensing sub-units, each located on substantially opposite ends of the device. In addition, the applications of the ingestible device are not limited merely to gathering data, sampling and testing portions of the gastrointestinal tract, or delivering medicament. For example, in some embodiments the ingestible device may be adapted to include a number of chemical, electrical, or optical diagnostics for diagnosing a number of diseases. Similarly, a number of different sensors for measuring bodily phenomenon or other physiological qualities may be included on the ingestible device. For example, the ingestible device may be adapted to measure elevated levels of certain analytes, chemical compounds or impurities in the gastrointestinal tract, or the combination of localization, sampling, and appropriate diagnostic and assay techniques incorporated into a sampling chamber may be particularly well suited to determine the presence of small intestinal bacterial overgrowth (SIBO). It is also noted that although embodiments described herein focus on an ingestible device in the GI tract, such ingestible device described in FIGS. 1-64 may be used for delivering substances including medicaments and therapeutics in other parts of the body, such as but not limited to the female reproductive tract, and/or the like.

The various embodiments of systems, processes and apparatuses have been described herein by way of example only. It is contemplated that the features and limitations described in any one embodiment may be applied to any other embodiment herein, and flowcharts or examples relating to one embodiment may be combined with any other embodiment in a suitable manner, done in different orders, or done in parallel. It should be noted, the systems and/or methods described above may be applied to, or used in accordance with, other systems and/or methods. Various modifications and variations may be made to these example embodiments without departing from the spirit and scope of the embodiments, which is limited only by the appended embodiments. The appended embodiments should be given the broadest interpretation consistent with the description as a whole.

Implementations of the subject matter and the operations described in this specification can be implemented by digital electronic circuitry, or via computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus.

A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, discs, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., a FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical discs, or optical discs. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic discs, e.g., internal hard discs or removable discs; magneto optical discs; and CD ROM and DVD-ROM discs. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's user device in response to requests received from the web browser.

Implementations of the subject matter described in this specification can be implemented in a computing system that includes a back end component, e.g., as a data server, or that includes a middleware component, e.g., an application server, or that includes a front end component, e.g., a user computer having a graphical display or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include users and servers. A user and server are generally remote from each other and typically interact through a communication network. The relationship of user and server arises by virtue of computer programs running on the respective computers and having a user-server relationship to each other. In some implementations, a server transmits data (e.g., an HTML page) to a user device (e.g., for purposes of displaying data to and receiving user input from a user interacting with the user device). Data generated at the user device (e.g., a result of the user interaction) can be received from the user device at the server.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

For the purpose of this disclosure, the term “coupled” means the joining of two members directly or indirectly to one another. Such joining may be stationary or moveable in nature. Such joining may be achieved with the two members or the two members and any additional intermediate members being integrally formed as a single unitary body with one another or with the two members or the two members and any additional intermediate members being attached to one another. Such joining may be permanent in nature or may be removable or releasable in nature.

It should be noted that the orientation of various elements may differ according to other exemplary implementations, and that such variations are intended to be encompassed by the present disclosure. It is recognized that features of the disclosed implementations can be incorporated into other disclosed implementations.

### EXAMPLES

Experiment 1

Testing was performed on an ingestible device having the design depicted in FIGS. 12A, 12B and 13 and having within the sampling chambers Carwild sponges (IVALON, fiber-free PVAc), treated with preservatives. The sampling chambers were allowed to sample a simulated jejunum fluid analog containing 0.55% carboxymethyl-cellulose for 15 minutes at 37° C. Holes in the capsule were produced as described above in connection with FIGS. 10 and 11. After the 15 minute sample, the ingestible device was placed in a centrifuge mechanism as described above with respect to FIGS. 6 and 7 and centrifuged for 4 minutes at 929 g and 4° C.

Results are shown in Table 1.

TABLE 1

| | Mass of Eppendorf | Mass of Eppendorf | |
|---|---|---|---|
| Sample | tube without | tube with | Sample |
| Chamber | sample [g] | sample [g] | mass [g] |
| Right | 0.504 | 0.568 | 0.064 |
| Left | 0.505 | 0.572 | 0.067 |

With the assumption that the samples had approximately the same density as water, the results show that 644 and 674 were extracted from the right and left chambers of the capsule, respectively.

Experiment 2

Testing was performed using the lancing device described above with respect to FIG. 3 with a centrifuge mechanism as described above with respect to FIGS. 6 and 7. Three ingestible devices contained Porex sponges (PSU-567, PE/PET fibers, hydrophilic treatment) and were allowed to sample a solution containing biomarkers for 15 minutes at room temperature at each sampling chamber. The sampling fluid contained biomarkers to allow cross contamination and downstream contamination to be detected after the samples were extracted.

Table 2 provides the weight in grams of sample recovered from a given chamber.

TABLE 2

| Chamber | Device 1 | Device 2 | Device 3 |
|---|---|---|---|
| Left | 0.0815 | 0.0870 | 0.0579 |
| Right | 0.0761 | 0.0782 | 0.0803 |

Table 3 lists preservative and analyte information for the sampling chambers of the devices. “Center” refers to liquid outside of the device. This allowed for testing not only for cross-contamination between sampling chambers in the device, but also for cross-contamination between sampling chambers in the device and liquid outside the device.

TABLE 3

| Chamber | Left | Right | Center |
|---|---|---|---|
| Presevative | Roche Complete | Custom made | None |
| | (T4) | RNAlater (T9) | |
| Analyte | Calprotectin | RNA* | DNA |

*RNA recovery was not available because the RNA was diluted to accommodate the simulated capsule sampling resulting in diluted sample below appropriate detection limits.

Table 4 provides contamination-related information for the devices based on fluorimetry measurements.

TABLE 4

| Chamber | Device 1 | Device 2 | Device 3 |
|---|---|---|---|
| Left | 0% DNA** | 0% DNA** | 0% DNA** |
| | 100% | 100% | 100% |
| | Calprotectin | Calprotectin | Calprotectin |
| | (1.43 ng) | (1.27 ng) | (2.74 ng) |
| Right | 0% DNA** | 0% DNA** | 0% DNA** |
| | 0% Calprotectin | 0% Calprotectin | 3% Calprotectin |
| | (−0.048 ng***) | (−0.085 ng***) | (0.082 ng) |
| Center | 100% DNA | 100% DNA | 100% DNA |
| | (5754.52 ng) | (6090.75 ng) | (5406.00 ng) |
| | 0% Calprotectin | 0% Calprotectin | 0% Calprotectin |
| | (−0.007 ng**) | (−0.016 ng**) | (−0.008 ng**) |

**Below the detection limit of 0.50 ng/mL the Qubit fluorimeter used.
***Negative value based on extrapolation of standard curve

In Device 1, no contamination was observed.

In Device 2, no contamination was observed.

In Device 3, the sum of contaminations was 3%.

Experiment 3

An ingestible medical device according to the disclosure (“TLC1”) was tested on 20 subjects to investigate its localization ability. TLC1 was a biocompatible polycarbonate capsule that contained a power supply, electronics and software. An onboard software algorithm used time, temperature and reflected light spectral data to determine the location of the capsule as it traveled the GI tract. The capsule is 0.51×1.22 inches which is larger than a vitamin pill which is 0.4×0.85 inches. The subjects fasted overnight before participating in the study. Computerized tomography (“CT”) were used as a basis for determining the accuracy of the localization data collected with TLC1. One of the 20 subjects did not follow the fasting rule. CT data was lacking for another one of the 20 subjects. Thus, these two subjects were excluded from further analysis. TLC1 sampled RGB data (radially transmitted) every 15 seconds for the first 14 hours after it entered the subject's stomach, and then samples every five minutes after that until battery dies. TLC1 did not start to record optical data until it reached the subject's stomach. Thus, there was no RGB-based data for the mouth-esophagus transition for any of the subjects.

In addition, a PillCam® SB (Given Imaging) device was tested on 57 subjects. The subjects fasted overnight before joining the study. PillCam videos were recorded within each subject. The sampling frequency of PillCam is velocity dependent. The faster PillCam travels, the faster it would sample data. Each video is about seven to eight hours long, starting from when the capsule was administrated into the subject's mouth. RGB optical data were recorded in a table. A physician provided notes on where stomach-duodenum transition and ileum-cecum transition occurred in each video. Computerized tomography (“CT”) was used as a basis for determining the accuracy of the localization data collected with PillCam.

Esophagus-Stomach Transition

For TLC1, it was assumed that this transition occurred one minute after the patient ingested the device. For PillCam, the algorithm was as follows:
1. Start mouth-esophagus transition detection after capsule is activated/administrated
2. Check whether Green <102.3 and Blue <94.6
   a. If yes, mark as mouth-esophagus transition
   b. If no, continue to scan the data
3. After detecting mouth-esophagus transition, continue to monitor Green and Blue signals for another 30 seconds, in case of location reversal
   a. If either Green >110.1 or Blue >105.5, mark it as mouth-esophagus location reversal
   b. Reset the mouth-esophagus flag and loop through step 2 and 3 until the confirmed mouth-esophagus transition detected
4. Add one minute to the confirmed mouth-esophagus transition and mark it as esophagus-stomach transition

For one of the PillCam subjects, there was not a clear cut difference between the esophagus and stomach, so this subject was excluded from future analysis of stomach localization. Among the 56 valid subjects, 54 of them have correct esophagus-stomach transition localization. The total agreement is 54/56=96%. Each of the two failed cases had prolonged esophageal of greater than one minute. Thus, adding one minute to mouth-esophagus transition was not enough to cover the transition in esophagus for these two subjects.

Stomach-Duodenum

For both TLC1 and PillCam, a sliding window analysis was used. The algorithm used a dumbbell shape two-sliding-window approach with a two minute gap between the front (first) and back (second) windows. The two minute gap was designed, at least in part, to skip the rapid transition from stomach to small intestine and capture the small intestine signal after capsule settles down in small intestine. The algorithm was as follows:
1. Start to check for stomach-duodenum transition after capsule enters stomach
2. Setup the two windows (front and back)
   a. Time length of each window: 3 minutes for TLC1; 30 seconds for PillCam
   b. Time gap between two windows: 2 minutes for both devices
   c. Window sliding step size: 0.5 minute for both devices
3. Compare signals in the two sliding windows
   a. If difference in mean is higher than 3 times the standard deviation of Green/Blue signal in the back window
   i. If this is the first time ever, record the mean and standard deviation of signals in the back window as stomach reference
   ii. If mean signal in the front window is higher than stomach reference signal by a certain threshold (0.3 for TLC1 and 0.18 for PillCam), mark this as a possible stomach-duodenum transition
b. If a possible pyloric transition is detected, continue to scan for another 10 minutes in case of false positive flag
   i. If within this 10 minutes, location reversal is detected, the previous pyloric transition flag is a false positive flag. Clear the flag and continue to check
   ii. If no location reversal has been identified within 10 minutes following the possible pyloric transition flag, mark it as a confirmed pyloric transition
c. Continue monitoring Green/Blue data for another 2 hours after the confirmed pyloric transition, in case of location reversal
   i. If a location reversal is identified, flag the timestamp when reversal happened and then repeat steps a-c to look for the next pyloric transition
   ii. If the capsule has not gone back to stomach 2 hours after previously confirmed pyloric transition, stops location reversal monitoring and assume the capsule would stay in intestinal area

For TLC1, one of the 18 subjects had too few samples (<3 minutes) taken in the stomach due to the delayed esophagus-stomach transition identification by previously developed localization algorithm. Thus, this subject was excluded from the stomach-duodenum transition algorithm test. For the rest of the TLC1 subjects, CT images confirmed that the detected pyloric transitions for all the subjects were located somewhere between stomach and jejunum. Two out of the 17 subjects showed that the capsule went back to stomach after first the first stomach-duodenum transition. The total agreement between the TLC1 algorithm detection and CT scans was 17/17=100%.

For one of the PillCam subjects, the capsule stayed in the subject's stomach all the time before the video ended. For another two of the PillCam subjects, too few samples were taken in the stomach to run the localization algorithm. These three PillCam subjects were excluded from the stomach-duodenum transition localization algorithm performance test. The performance summary of pyloric transition localization algorithm for PillCam was as follows:
1. Good cases (48 subjects):
   a. For 25 subjects, our detection matches exactly with the physician's notes
   b. For 19 subjects, the difference between the two detections is less than five minutes
   c. For four subjects, the difference between the two detections is less than 10 minutes (The full transition could take up to 10 minutes before the G/B signal settled)
2. Failed cases (6 subjects):
   a. Four subjects had high standard deviation of Green/Blue signal in the stomach
   b. One subject had bile in the stomach, which greatly affected Green/Blue in stomach
   c. One subject had no Green/Blue change at pyloric transition

The total agreement for the PillCam stomach-duodenum transition localization algorithm detection and physician's notes was 48/54=89%.

Duodenum-Jejenum Transition

For TLC1, it was assumed that the device left the duodenum and entered the jejenum three minutes after it was determined that the device entered the duodenum. Of the 17 subjects noted above with respect to the TLC1 investigation of the stomach-duodenum transition, 16 of the subjects mentioned had CT images that confirmed that the duodenum jejenum transition was located somewhere between stomach and jejunum. One of the 17 subjects had a prolonged transit time in duodenum. The total agreement between algorithm detection and CT scans was 16/17=94%.

For PillCam, the duodenum-jejenum transition was not determined.

Jejenum-Ileum Transition

It is to be noted that the jejunum is redder and more vascular than ileum, and that the jejenum has a thicker intestine wall with more mesentery fat. These differences can cause various optical responses between jejunum and ileum, particularly for the reflected red light signal. For both TLC1 and PillCam, two different approaches were explored to track the change of red signal at the jejunum-ileum transition. The first approach was a single-sliding-window analysis, where the window is 10 minutes long, and the mean signal was compared with a threshold value while the window was moving along. The second approach was a two-sliding-window analysis, where each window was 10 minutes long with a 20 minute spacing between the two windows. The algorithm for the jejunum-ileum transition localization was as follows:
1. Obtain 20 minutes of Red signal after the duodenum-jejenum transition, average the data and record it as the jejunum reference signal
2. Start to check the jejunum-ileum transition 20 minutes after the device enters the jejunum
   a. Normalize the newly received data by the jejunum reference signal
   b. Two approaches:
      i. Single-sliding-window analysis
         Set the transition flag if the mean of reflected red signal is less than 0.8
   ii. Two-sliding-window analysis:
      Set the transition flag if the mean difference in reflected red is higher than 2× the standard deviation of the reflected red signal in the front window

For TLC1, 16 of the 18 subjects had CT images that confirmed that the detected jejunum-ileum transition fell between jejunum and cecum. The total agreement between algorithm and CT scans was 16/18=89%. This was true for both the single-sliding-window and double-sliding-window approaches, and the same two subjects failed in both approaches.

The performance summary of the jejunum-ileum transition detection for PillCam is listed below:
1. Single-sliding-window analysis:
   a. 11 cases having jejunum-ileum transition detected somewhere between jejunum and cecum
   b. 24 cases having jejunum-ileum transition detected after cecum
   c. 19 cases having no jejunum-ileum transition detected
   d. Total agreement: 11/54=20%
2. Two-sliding-window analysis:
   a. 30 cases having jejunum-ileum transition detected somewhere between jejunum and cecum
   b. 24 cases having jejunum-ileum transition detected after cecum
   c. Total agreement: 30/54=56%

Ileum-Cecum Transition

Data demonstrated that, for TLC1, mean signal of reflected red/green provided the most statistical difference before and after the ileum-cecum transition. Data also demonstrated that, for TLC1, the coefficient of variation of reflected green/blue provided the most statistical contrast at ileum-cecum transition. The analysis based on PillCam videos showed very similar statistical trends to those results obtained with TLC1 device. Thus, the algorithm utilized changes in mean value of reflected red/green and the coefficient of variation of reflected green/blue. The algorithm was as follows:
1. Start to monitor ileum-cecum transition after the capsule enters the stomach
2. Setup the two windows (front (first) and back (second))
   a. Use a five minute time length for each window
   b. Use a 10 minute gap between the two windows
   c. Use a one minute window sliding step size
3. Compare signals in the two sliding windows
   a. Set ileum-cecum transition flag if
      i. Reflected red/green has a significant change or is lower than a threshold
      ii. Coefficient of variation of reflected green/blue is lower than a threshold
   b. If this is the first ileum-cecum transition detected, record average reflected red/green signal in small intestine as small intestine reference signal
   c. Mark location reversal (i.e. capsule returns to terminal ileum) if
      i. Reflected red/green is statistically comparable with small intestine reference signal
      ii. Coefficient of variation of reflected green/blue is higher than a threshold
   d. If a possible ileum-cecum transition is detected, continue to scan for another 10 minutes for TLC1 (15 minutes for PillCam) in case of false positive flag
      i. If within this time frame (10 minutes for TLC1, 15 minutes for PillCam), location reversal is detected, the previous ileum-cecum transition flag is a false positive flag. Clear the flag and continue to check
      ii. If no location reversal has been identified within this time frame (10 minutes for TLC1, 15 minutes for PillCam) following the possible ileum-cecum transition flag, mark it as a confirmed ileum-cecum transition
   e. Continue monitoring data for another 2 hours after the confirmed ileum-cecum transition, in case of location reversal
      i. If a location reversal is identified, flag the timestamp when reversal happened and then repeat steps a-d to look for the next ileum-cecum transition
      ii. If the capsule has not gone back to small intestine 2 hours after previously confirmed ileum-cecum transition, stop location reversal monitoring and assume the capsule would stay in large intestinal area

The flag setting and location reversal criteria particularly designed for TLC1 device were as follows:
1. Set ileum-cecum transition flag if
   a. The average reflected red/Green in the front window is less than 0.7 or mean difference between the two windows is higher than 0.6
   b. And the coefficient of variation of reflected green/blue is less than 0.02
2. Define as location reversal if
   a. The average reflected red/green in the front window is higher than small intestine reference signal
   b. And the coefficient of variation of reflected green/blue is higher than 0.086

For TLC1, 16 of the 18 subjects had CT images that confirmed that the detected ileum-cecum transition fell between terminal ileum and colon. The total agreement between algorithm and CT scans was 16/18=89%. Regarding those two subject where the ileum-cecum transition localization algorithm failed, for one subject the ileum-cecum transition was detected while TLC1 was still in the subject's terminal ileum, and for the other subject the ileum-cecum transition was detected when the device was in the colon.

Among the 57 available PillCam endoscopy videos, for three subjects the endoscopy video ended before PillCam reached cecum, and another two subjects had only very limited video data (less than five minutes) in the large intestine. These five subjects were excluded from ileum-cecum transition localization algorithm performance test. The performance summary of ileum-cecum transition detection for PillCam is listed below:
1. Good cases (39 subjects):
   a. For 31 subjects, the difference between the PillCam detection and the physician's notes was less than five minutes
   b. For 3 subjects, the difference between the PillCam detection and the physician's notes was less than 10 minutes
   c. For 5 subjects, the difference between the PillCam detection and the physician's notes was less than 20 minutes (the full transition can take up to 20 minutes before the signal settles)
2. Marginal/bad cases (13 subjects):
   a. Marginal cases (9 subjects)
      i. The PillCam ileum-cecum transition detection appeared in the terminal ileum or colon, but the difference between the two detections was within one hour
   b. Failed cases (4 subjects)
      i. Reasons of failure:
         1. The signal already stabilized in the terminal ileum
         2. The signal was highly variable from the entrance to exit
         3. There was no statistically significant change in reflected red/green at ileum-cecum transition

The total agreement between ileocecal transition localization algorithm detection and the physician's notes is 39/52=75% if considering good cases only. Total agreement including possibly acceptable cases is 48/52=92.3%

Cecum-Colon Transition

Data demonstrated that, for TLC1, mean signal of reflected red/green provided the most statistical difference before and after the cecum-colon transition. Data also demonstrated that, for TLC1, the coefficient of variation of reflected bluee provided the most statistical contrast at cecum-colon transition. The same signals were used for PillCam. The cecum-colon transition localization algorithm was as follows:
1. Obtain 10 minutes of reflected red/green and reflected blue signals after ileum-cecum transition, average the data and record it as the cecum reference signals
2. Start to check cecum-colon transition after capsule enters cecum (The cecum-colon transition algorithm is dependent on the ileum-cecum transition flag)
   a. Normalize the newly received data by the cecum reference signals
   b. Two-sliding-window analysis:
      i. Use two adjacent 10 minute windows
      ii. Set the transition flag if any of the following criteria were met
         The mean difference in reflected red/green was more than 4× the standard deviation of reflected red/green in the back (second) window
         The mean of reflected red/green in the front (first) window was higher than 1.03
         The coefficient of variation of reflected blue signal in the front (first) window was greater than 0.23

The threshold values above were chosen based on a statistical analysis of data taken by TLC1.

For TLC1, 15 of the 18 subjects had the cecum-colon transition detected somewhere between cecum and colon. One of the subjects had the cecum-colon transition detected while TLC1 was still in cecum. The other two subjects had both wrong ileum-cecum transition detection and wrong cecum-colon transition detection. The total agreement between algorithm and CT scans was 15/18=83%.

For PillCam, for three subjects the endoscopy video ended before PillCam reached cecum, and for another two subjects there was very limited video data (less than five minutes) in the large intestine. These five subjects were excluded from cecum-colon transition localization algorithm performance test. The performance summary of cecum-colon transition detection for PillCam is listed below:
1. 27 cases had the cecum-colon transition detected somewhere between the cecum and the colon
2. one case had the cecum-colon transition detected in the ileum
3. 24 cases had no cecum-colon transition localized

The total agreement: 27/52=52%.

The following table summarizes the localization accuracy results.

| | | |
|---|---|---|
| Transition | TLC1 | PillCam |

| | | | |
|---|---|---|---|
| Stomach-Duodenum | 100% | (17/17) | 89% (48/54) |
| Duodenum-Jejenum | 94% | (16/17) | N/A |
| Ileum-Cecum | 89% | (16/18) | 75% (39/52) |
| Ileum-terminal | 100% | (18/18) | 92% (48/52) |
| ileum/cecum/colon | | | |

### Other Embodiments

While certain embodiments have been described, the disclosure is not limited to such embodiments. For illustrative purposes, the examples provided by this disclosure focus primarily on a number of example embodiments of ingestible devices, sleeve devices, centrifuge mechanisms, adaptor devices, lancing devices, grating devices, and cutting devices. However, it will be understood that modifications to the general shape and structure of the various devices and mechanisms described in relation to FIGS. 1-31 may be made without significantly changing the functions and operations of the devices and mechanisms. Furthermore, it should be noted that the features and limitations described in any one embodiment may be applied to any other embodiment herein, and the descriptions and examples relating to one embodiment may be combined with any other embodiment in a suitable manner. For example, sleeve devices disclosed herein may be interchangeable with one another, and the teachings discussed in relation to any one sleeve device may be applied to any other in an appropriate fashion. Similarly, any of the sleeve devices disclosed herein, or exemplary embodiments of adapters disclosed herein, may be incorporated into any systems and methods for centrifuging an ingestible device, or portion of an ingestible device, such as the systems and methods described in relation to FIG. 1, FIG. 6, and FIG. 7. Moreover, the figures and examples provided in disclosure are intended to be only exemplary, and not limiting. It should also be noted that the systems and/or methods described above may be applied to, or used in accordance with, other systems and/or methods, including systems and/or methods that may or may not be directly related to ingestible devices.

1. A method for extracting a sample from an ingestible device, the ingestible device having a housing with an opening connected to a sampling chamber of the ingestible device, the method comprising:
centrifuging the ingestible device to transfer at least a portion of a sample contained in the sampling chamber out of the ingestible device and into a tube via the opening.

2. The method of claim 1, further comprising connecting the opening to the tube via a sleeve.

3. The method of claim 2, further comprising inserting at least a portion of the ingestible device into the sleeve.

4. The method of claim 2, wherein the sleeve comprises a fitted sleeve.

5. The method of claim 2, wherein the ingestible device is centrifuged while the ingestible device is at least partially inserted into the sleeve.

6. (canceled)

7. The method of any of claim 1, further comprising exposing the ingestible device to a heated lancet to generate the opening.

8. The method of any of claim 1, further comprising sliding a grating surface against the ingestible device to remove a portion of the housing of the ingestible device to generate the opening.

9. The method of claim 1, wherein:
the ingestible device has a second opening connected to a second sampling chamber of the ingestible device; and
the method further comprises centrifuging the ingestible device to transfer at least a portion of a second sample contained in the second sampling chamber into a second tube via the second opening.

10. The method claim 1, wherein the sampling chamber further comprises an absorbent material, and the sample is at least partially absorbed by the absorbent material prior to centrifuging.

11. The method of claim 1, wherein centrifuging comprises:
inserting at least a portion of the ingestible device into a sleeve;
while at least the portion of the ingestible device is inserted into the sleeve, inserting the ingestible device and the sleeve into a centrifuge tube;
attaching the centrifuge tube to a cap having a spring on a bottom surface of the cap;
causing the spring to maintain a position of the ingestible device relative to the fitted sleeve as the ingestible device is at least partially inserted into the fitted sleeve; and
centrifuging the centrifuge tube while the ingestible device and the fitted sleeve are inserted into the centrifuge tube.

12.-13. (canceled)

14. The method of claim 1, wherein the sample is obtained by the ingestible device from a predetermined region of the gastrointestinal tract.

15. The method of claim 1, wherein the method results in less than 5% sample contamination.

16. A sleeve device configured to extract a sample from an ingestible device comprising a housing with a first end having a device opening that leads to a sampling chamber containing the sample, the sleeve device comprising:
a sleeve portion configured to fit snugly around a circumference of the ingestible device; and
a base portion comprising:
a first surface with a first opening;
a second surface with a second opening; and
a tunnel connecting the top and second openings,
wherein:
the first surface is connectable to the sleeve portion; and
the first surface comprises a seal portion configured to fit the first end of the ingestible device and to provide a liquidtight seal between the first opening and the device opening.

17.-28. (canceled)

29. A method, comprising:
separating an ingestible device into a first portion and a second portion, the first portion comprising a sampling chamber that contains a sample; and
centrifuging the first portion of the ingestible device to transfer at least a portion of the sample from the sampling chamber into the tube.

30. The method of claim 29, further comprising, prior to centrifuging, connecting the sampling chamber to the tube via an adapter.

31. The method of claim 29, wherein separating the ingestible device into the first and second portions comprises:
inserting the ingestible device into a cutting device having a recessed area and a moveable blade such that the ingestible device is positioned within the recessed area; and
causing the moveable blade to cut the ingestible device to form the first portion and the second portion while the recessed area holds the ingestible device.

32.-41. (canceled)

42. An adapter device having a first surface with a first opening, a second surface with a second opening, and a body between the first and second surfaces, the body having a tunnel that connects the first and second openings, the adapter device comprising:
a hollow needle protruding from the second surface and having a lumen connected to the second opening to define a fluid channel from the lumen, the second opening, the tunnel, and the first opening,
wherein the hollow needle is configured to enter a sampling chamber within an ingestible device to allow at least a portion of a sample in the ingestible device to pass through the fluid channel.

43. (canceled)

50. A grating device configured to remove a portion of a housing of an ingestible device to expose a sampling chamber containing a sample within the ingestible device, the grating device comprising:
a holder comprising a rail and a recessed area sized and configured to snugly hold a first portion of an ingestible device while exposing a second portion of the ingestible device;
a slide block configured to move along the rail and pass over the second portion of the ingestible device; and
a grating surface on the slide block and positioned so that when the slide block passes over the second portion of the ingestible device, the grating surface scrapes against the second portion of the ingestible device to remove a portion of the housing of the ingestible device and expose the sampling chamber.

51.-63. (canceled)

63. A method, comprising:
removing a sample from an ingestible device so that there is less than 5% sample contamination.

64.-68. (canceled)

69. The method of claims 63, comprising removing at least two different samples from the ingestible device, wherein there is less than 5% contamination of either sample.

70.-86. (canceled)
